SEQUENCE LISTING (40 sequences)

SEQ ID NO: 1 - DNA, 980 bp, Homo sapiens
tttctcgagagaggcaaggcagcctacatgagtcctgggctgcaggaggctctaggaaccctggggcctgagactgaggtccagggagaccctaattcctgcaccccacccctcctggttccctccagatgggaggcatggaggctgtcatcacgggcctggcagatgacttccaggtcctgaagcgacaccggaaactcttcacatttggcgtcaccttcagcactttccttctcgccctgttctgcataaccaaggtgagtaggggctgggctctgggtcacctgggggcctctgaggccgcatttcaataaagtcaaacattcctagccttagaactgggctgagctcagggagaacaatgcaggatccagcatcctcaattcagcggcctgacccactagggttaggcccagtagtcttcttccatctctgassctgaggattccattcagccctgttaattgccttattgacttgagggscagcaaaagtccctttggaacccatctaactctttattggctgaaactgaggtgactgtaacgtcaatacaacagcaccacagccctatgccctgggttttcaaatagagctccgagcaagtgggacagggggcaggtaagagttgacagacacaacaatcagttcccacgtttgaccaaagagggcctcttggcttcttctctccctgtgccagggtggaatttacgtcttgaccctcctggacacctttgctgcgggcacctccatcctttttgctgtcctcatggaagccatcggagtttcctggttttatggtatgtgagtgtgtggaaaagcctcagctcccagtcctcctagaatcctgcacctggaggtgtgcagggaggccttccatttccaggacagccacctaaaattccagagtccagcaagtcacttattgggaacaaatctcaatcctcggctcatctttggatgaacctgcccttaacagg

SEQ ID NO: 2 - DNA, 4532 bp, Homo sapiens
  misc_feature: N = any nucleotide
agctcaagaaacagtgtctggatgagtgactcaatggaccagctccacaaacaaagctggaggtgtcttgtacagaccccaaatgctatccatgtggggctgcaggatcaaatagcaggtggccctcatctgcaggtgcagccaggctgcragaagggtgtccctgggccaagctgaggcctcctccccttctcttcctttcagagactggcctatggcatcacgccagagaacgagcaccacctggtggctcagagggacatcagacagttccaggtgggtgaagcctagacccctggggtggagattacaagggcgggccctggctgttccctgctgtgtactgcccaaggctagacatcacatccagaaaacccagaaacccagtgtgagctgccttttccccttggaaacatcgggatgggggacagggaggctcaccttgagcccatggcctcaggcttgccctgtgactttggggaggttctgctgccctttctgggcctctgtgacaattagggaatcaacttgcacgttccctgaggtccgtgaaggaagggggtgnttttctgccttctctctacctcctgctgcccccgccagctggcccttgctcctttctgtccccaccatgtcatcaagncctcgctgtctttctctgcagttgcaacactggctggccatctgagcctgcctggaggagaaggaggaacccccatgccaatgtccaggtcacaggcatccgctgcgctcccacctcggacaccatcttgggattcctcccctggaagttgtcctttctgatcctctcttcttttcccatttacaaatgatttcgtgactgtagtttttgttcaccttctgtgcatctggcctgggggctgttagctcagaggagaggagcaaacaggaaaatgacttctgttctgtccccgctgttttgggggaagtctctcccactttgggatcctgctgaagctaggttcatgaggtcggaaatccccaccacatttgcctagactttgggcacaggagttcttagtccaccaaatcagagagaggatgggcttttgatcagatacccctcccaaaaaaaaaaaaaaactaaaactaaagcaaaaatcaaamaaaatctggctgagttttagtggggtggttggggaaggtacatagaccctcctcttgcccaccctagacagccctctcatgtctgaacctcagcctgggagttagatttatttgtctctaaaatgaagtcagtggatagatgctttgagggattttgagtagaaacattcatagttaatnktcactctggccaatctgagtttgatgtgtgtgttctggaacattcctccagcttttggtggtcagatggcccagagatatgggggacaggaggaagagggtaaatgaaccacagtgagcaggttctaggaggtacctgcatcagacaagctggtggaggccacgtggcaagccacatctactgaggcctcatgctgctcttgctctgtaagacacggagcccagaaacccatctgcacttcctgagacctgcctggggaaacgggggcagggaccaagtgaggcctcatgtgtgtcttcaccgtgctgtcctcacaaggccaggtgggtgcccaaagggagcctgacaggctgttgtgttaatttattgttcttgcacacctgcacagcctccctctggggatcccacctggagtggaccaggggtcttgagaaatggagagttggctgcaaaaactctcatgcactagatgtggcaccttggagggcagggtgagacaagcagcccagaaatactctctcaagtggaggggagaattttgagagtggatggaacagtttggtggtttcagagaatttctaggtttctacttggatctacttctgatacaaacttgcacttggtgccctctggtggtgtttagttttagttccgtaagagaaatgattcctagtttgctaaattggtggcatctttgggaggggtttctgtttatggttagagtctcttacacccttgttggagggattcttattctgactgtgggagctcctgttgcaggatcttgggaaaaaataaagaagccgctgcattcgcacgtcaagaaggtgctttgcctcaaattggggtgttgttgagcctggtggttcctgcatgaagaggattatgaggggaccagggtggggcagggagatggttttgtctcccagggtcctgaggtttccttgctgggtcggggtcctcaggtcattctatagataaaagagggaaaatcaggagactttgaatcttntctgtataaaanaggnncagckgaatgcctgagacagcccnggtggcaggtgtcttgagccctgtgaacagtgaggcttaagaatggagaacaatcaggtcggggtctgggccccattagtgactttatatcctcccataaaaggtaacttcttcctaggtgttaccatttttcttttcgttttttgtttttgtttttgtttgtttgtttgtttgtttgtaataaagcactttaatgcacattacctgccatctgcccagttgaggactgcagggctatagcttctatctccccatttcccaggtgagagaaccaaggcccagcattttagtcactcttgctcaaggttttttagcaactaacagatcgagttgggccttcaattcacatccactgactctcagcccagattattttgaatattccctgcacaatagggtcaccccacccaggactgtcatttttaaaaaactcattcaaaccgcaaaggaaaatttcttagcaaaagaacaatgtgttggaggatgggaagggcgagagaatgccatttattttcctctagctggtttccagagaggaaattatttagctgctctcttttgatgaaaataatcactctttggaatagttggatgtgaaaagctgagtctacttggttgaaatgagagcamacagtcagcaaagccttttgtattagagcagggtcgtgcttccgagagagcctgccatttcctctttgccatctgcatgtggccccttctgcctccagacatttgtcccggggtgaatcggagatgtggtgctagctgaaccaacaccaaaccaangcagtggtgctgccttgcgrcggaagttggctcctgaatctgggatgggaacctggctccaagcctgggtcccctgggagggtgggggtacccagaaagcccttggaagttctcgggaggtgcttggagatcatttgggtttacctttccacccacatttaatggagagagagtatgggctttatgttaagtcatctttgacttcctttttgtagcttgtttttaatagcagaggtcacccgggacaagggtgctgtgtactgtatatgacacttgacgcttttgatattttttcaggtttttaaagaattattatttttcatgaaatgtaaaatatcagtttgagaacatcacatttacgtctactcaatgtctagttatttagcacccaccttttagctttcattctagatgaaaacgagacaagggagaaggagagctaccaactcttgccagatatcctgctgaacagaaatccctgaagctgccttaattctcaaaaggagttaccgctcagtgggagccagttcctgctatatgatctgttttctagcctcgctaatgtgagactgaagcattcttaccaaagaaatcatttcctagtaaagaagcccattgaactcactttatttgtttatttccttcgaaagccaccgaagagagaaraacagagaaggtgtgtagtgtggcggaaaaagcacagcatatagttttacagacttgggtctgcagctgactagctgggtggcctggggatagtggcttcatcttttggggcttcaagattctttgtctttaaaatcaggggttatatcagatcatcaaagttcccattccattaaagaaaaccctgcatgtatccataatgatgctckcctgttaaatttacaatgaaggaaacacatcacttaactgtaagaatttcccaaaatgaactgatgaccagtgatctctctatcagagaaatgtcagatttctagcctccagaactttgatttttctggacattcaatagttcctctttctcagatatttttcaactgatgccagaaacacttggtatttgtttttaatccaacccttttgttttgaggctttggagggtaacacattttcataccctgtgagtcccaggatcacagctctgctgtggtcttagaagccactgaaacattggtgaatgtgaagtcacttttggggtgcctgccctcatccctctgtctctctgcttccgtgtaaataaagactgtttcaattgtgtcctctctgtgtcatggactgttccaatgtcatgcagatttctgtgtctgatatggattttaaaagttgttctctttgtggaatataagtgtacagaataataaataatgtttcctgggctgtg

SEQ ID NO: 3 - DNA, 395 bp, Homo sapiens
tctgttatctctaaacctgtgttctgtccgcccacacatgacctaacaattgggcccccagatactcccctatcatgtgcagctcagaccaatggtttcagccattgatgaggtccttgctgtttcttacaggagctggcctagtgttcatcctgtatccagaggccatttctaccctgtctggatctacattctgggctgttgtgtttttcgtcatgctcctggcgctgggccttgacagctcagtgagtgaccctgcttaggatacctatcccccatcccactgggcctgacccccttccccaacacacagtgctgggcctgaagttcccactattcaaacaccaggttaacagttgtttcccagaaggccctatttaaattgcagacaaaaa

SEQ ID NO: 4 - DNA, 3946 bp, Homo sapiens
accgctccggagcgggaggggaggcttcgcggaacgctctcggcgccaggactcgcgtgcaaagcccaggcccgggcggccagaccaagagggaagaagcacagaattcctcaactcccagtgtgcccatgagtaagagcaaatgctccgtgggactcatgtcttccgtggtggccccggctaaggagcccaatgccgtgggcccgaaggaggtggagctcatccttgtcaaggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggcggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctctacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactccctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcagcctgggccgtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcctgcaatgggagagacacgaacaaaccaaggaaatctaagtttcgagagaaaggagggcaacttctactcttcaacctctactgaaaacacaaacaacaaagcagaagactcctctcttctgactgtttacacctttccgtgccgggagcgcacctcgccgtgtcttgtgttgctgtaataacgacgtagatctgtgcagcgaggtccaccccgttgttgtccctgcagggcagaaaaacgtctaacttcatgctgtctgtgtgaggctccctccctccctgctccctgctcccggctctgaggctgccccaggggcactgtgttctcaggcggggatcacgatccttgtagacgcacctgctgagaatccccgtgctcacagtagcttcctagaccatttactttgcccatattaaaaagccaagtgtcctgcttggtttagctgtgcagaaggtgaaatggaggaaaccacaaattcatgcaaagtcctttcccgatgcgtggctcccagcagaggccgtaaattgagcgttcagttgacacattgcacacacagtctgttcagaggcattggaggatgggggtcctggtatgtctcaccaggaaattctgtttatgttcttgcagcagagagaaataaaactccttgaaaccagctcaggctactgccactcaggcagcctgtgggtccttgtggtgtagggaacggcctgagaggagcgtgtcctatccccggacgcatgcagggcccccacaggagcgtgtcctatccccggacgcatgcagggcccccacaggagcatgtcctatccctggacgcatgcagggcccccacaggagcgtgtactaccccagaacgcatgcagggcccccacaggagcgtgtactaccccaggacgcatgcagggcccccactggagcgtgtactaccccaggacgcatgcagggcccccacaggagcgtgtcctatccccggaccggacgcatgcagggcccccacaggagcgtgtactaccccaggacgcatgcagggcccccacaggagcgtgtactaccccaggatgcatgcagggcccccacaggagcgtgtactaccccaggacgcatgcagggcccccatgcaggcagcctgcagaccaacactctgcctggccttgagccgtgacctccaggaagggaccccactggaattttatttctctcaggtgcgtgccacatcaataacaacagtttttatgtttgcgaatggctttttaaaatcatatttacctgtgaatcaaaacaaattcaagaatgcagtatccgcgagcctgcttgctgatattgcagtttttgtttacaagaataattagcaatactgagtgaaggatgttggccaaaagctgctttccatggcacactgccctctgccactgacaggaaagtggatgccatagtttgaattcatgcctcaagtcggtgggcctgcctacgtgctgcccgagggcaggggccgtgcagggccagtcatggctgtcccctgcaagtggacgtgggctccagggactggagtgtaatgctcggtgggagccgtcagcctgtgaactgccaggcagctgcagttagcacagaggatggcttccccattgccttctggggagggacacagaggacggcttccccatcgccttctggccgctgcagtcagcacagagagcggcttccccattgccttctggggagggacacagaggacagtttccccatcgccttctggttgttgaagacagcacagagagcggcttccccatcgccttctggggaggggctccgtgtagcaacccaggtgttgtccgtgtctgttgaccaatctctattcagcatcgtgtgggtccctaagcacaataaaagacatccacaatggaaaaaaaaaaaggaattc

SEQ ID NO: 5 - DNA, 740 bp, Homo sapiens
ggatttactttgcaggcaccgagactgccacacactggagcggctacatggagggggctgtagaggccggggagagagcagcccgagaggtagggtctggggtcatagattatggggaaggaggcaactcatggtcctgacatgtaggtcaaagatatctgagagcctggagaccaactctccagtaacccccatcttaagacccataaaatactattttaactattgtctcaggtgaagtgaacaccttagcccgaagtaataatgaaaatacctaacatgtcacccgaagtaaaaagagcccatggggcaaacgatcaacctgactatacttgaaaggtaacctaggtctgatattctagaaccaaacatgcttctattaggactcaaaattcaattcatttcacaggaaagattttctgacagttcctctgatgtcatccctatttgaggtgtgttcataactattaaaatttatggtaagcctaatgattggaacctcttataccacaggagaaagaccttttggcgcctcttaaaggttgtatggagtgttctggcctttaccttggtgtttttgatggatatctcaagagacagttacttagtcctttagggagcagattagaagaaagatggtgtcgcttttgctatttgccagtgtgttctttcatttcagatcctgcatgccatggggaagattccagaggatgaaatctggcagtcagaaccagagtctg

SEQ ID NO: 6 - DNA, 33654 bp, Homo sapiens
  misc_feature: N = A, C, T or G
gggaatgagtctccatttcttctgcnatntgctgaggttgcctcctcggccagagaacataaaagtgttattcagtaactctctccttttcccttggatgtgggaaggaacgggcgcaccacctttcctccctcttccatttccatgctgcctttttgaagttgcaagagatgctaaaagaaggtccaaccccaagtgagagcctggcctttgcctggaggggtaggggcaggtggcctatatcgccagggtcctggaaaggaacctcttacccactcactcccaattcctatccaactcaagtcactccagtaggctgcttttgagatgcaaatacaaaatgctttcttctccccctctactttttcattgaaatgctttgcatctcagaaaagaggaaaaaatgatgtgtttaagacaggagtgtctcacccgccatcaagcctgataaggaattgttaaattttaattagacaatttgattccttcaagtgccgaactttcagggaaggcagcaccaagacttaagctgagacggaagcataggaaacacgttgctggggtttatgctgcatccagggcttctgtcccccacctttctagctctgtgccccagatgaaatagaaatgaggtggtggggatagagggagggagagagagagagagagagagagagagagagagagagagagagaaagagaggatcagagaaaatcctctctgttctcccaggacctgccagctttcttcctggatcccaggctgtcctaacagattgacccttttcaccccactccatcagcctggggactaagtgttgctacaccttccaagtttctcctgagagaaaatgtcaggtttgggtgtcagcctcaaactgctgagccttaaaggagcttttttcccttccaatacactttataggttggggcgnctgccagttttggggcgtgactgggaaagcagncccttccatacctcatctataccccagaaaggactgcccactccctgtcccctcctctactcctggggggcagtgcagtgggtggcctgagcagggtggcctgcctggggcagcctgggtgggctagcttagccccgggtatctgggctcttccagtacttctcaacttgagagctagctaatttccaaattagggcacgcacagaaaatagtagcatttgtaaggcatatagggtgaatagcagaagctgctgaggttggaagtgcctgcctctacatcccaattgacctaatatttctatgcaccttgatgcattctagacctctgattggggaagttccactctgaagcttctgaagagagccactattatagaaggctggagttaggctagatgttaggaagaacttgacatgcaggggtcctgagcactagaatggatgactctgaaagatatttctgtagatctttgcccttctgagctagggcagagcaaagagtcctggagacagatgaggagatctttggctctttgatgaccagtagcttgcaggtcaccacacaaatgcagtcttctctttctcctggcttccaccagggcagctgagaaagaaaagcctaggaggcctcagaggacccatggaaccttgtctgcaagcatctgcatggtgagggtggaatgaggagcagcaattacccccactttcctgcacagaaaggtgccttaggggagaggcagccacccctggagtgaggcctggcagctacaggattagattgttcatcctccacccaggaaacactgtcaagtagctcaacctgtaacagctggaaaataacagtgtccatgcatgtatttggtgctcactaagttcttgttgactggggattgtgatttggagttttgtaagcttttatgcatgcccaggggccacctgggagaaagatggagatgacatttggagggtggtgagcaagttgatcatattttcacaatcaaaaatcaaaatgcttgttttgataacaatagatttttaaatatactgtgccactttatttgtctcaaaaaaatttctcatgtaagaataatgcaccttgggttatatatttagccantctccaytttaaaaaaagaacaagaaattgggaccatcatggcaaatacaaggtgccatgtgttatgtggctttgcattttccctcaacaagctccagagtggacaaagaactcaccaggcttctttggagccagaaccagcccagcgtaactggagcagcactgagaacctttcctcactccgctaccatgaacacaagctccagccaggatggagaagctactcacttgtgtattcttttgggatgccccttggagcccaacaatctgtgcagcccggagacagaaggacaaaagggagggatgggctggagctgcagcagggaagcctggggaccagcaggttttctcaggcctcttgagaaaataccagcttagaaatgctgggaaggccctgtattgctatccactctacaatgcacttcccmctgcccccaccattttaatgtctctgaaatcagaatgcatttkacaatggatgacatcttggagctctactggggttggatggcattwttttctttcctagtgatatatraaatagtggtgcatcgtcattcgattttgtcttagagtctatgaaatgtggtgattagtatgacagtcatcgrctatagcagctgggctgggagagctgtatcttccaggccagctgctacctcatctcaagggtcacttgagtttgtcctacagccagggcccagggacaggctttgtcttttccttccwttacttcctcccaaggagttggtggctgcatcttgaaatccatctcccttcatctagccacctatttttcaggatgggccataggaggcagtgggcccaggaggacctcagaccctagtgagctgcatttgaacagcaacagtcacttcatcttgctgagccgttggcttgtggtctgattgcttgtttcatttcattcttttgcttggtttgatttttaaagttgacattctcaagggcttacctgtgcctggcacatggccatgagtcatcttacttaatactcacaattgttgttatctctattttgtgtatgggaaaactgggctcaggaaagccaaataatctggctgaggccccaaagttagtaaatggtagagwtgggacttgaacccaggttttttagtcctcaactcgaatgttatactagcacctctcaggctagagtaccttctctgtggcagtggatttgctgatgtctgaattcctgctcagcttgatgtttacatggtgagagccagctaggctgcccctgcttgtgagaggtgaaagccccaagtagataaggtgctgaagacacgatagaaaccaggactgacaccaggtgttaaaagatagccctgtactggccgggtgcggtggctcacgcctgtaatcccagcactctgggaggccgaggcaggcagataatgaggtcaggagatcgagaccatcctgactaacacggtgaaaccccgtctctactaaaaatacaaaaaaattagctggacatggtggcgggcacctgtagtcctggctactcgggaggctgaggcaggagaatggcgtgaatccaggaggcggagcttgcagtgagccgagatagccccactgcactccagcccgggtgacagagcaagactccatctcaataacaaaaaaacccccaaacaagcaaacagacaaaaagatagccctgtaccaaggtacttataggggttacaggtatttaggcatttgccatgttttcccttttttttcctatggtgtaaatatactacgtttgtattagcaacaaaaagcaacgagaacaaccaataataaataatagattagaaaatcaaatgtgtgaggcctttctcagaacttgggtacagggactctcccttaccctgcagaagtaaccagcactccagagaatgaatgctcaccctcaaccccacctgacctgcagtgctcacatttgccatctacattgtactcataattttaaatgctagactagtgcctgtaatcccagcactttgggaggccaaggcaggagtatctcttgaggccaggagtgtgagaccagcctgggcaacatagtgagacccccatctctacaaaacatttttaaaaattagccaggcatcgtggcacatgcctgtatttccagctactcaggaggctgaggtggtaggatcacttgaggccaggagttgaggctgcagtgagctatgattgtgccactgcactccagcctgggtggcaaagtgggattctgtctctattaaaaaaaaattaaatcccagtttacatcctccctcctccaggaagttctccacaaacgccccagccacacacatgtgcatatgctcctgaacatcaactgtagtggacacggtggagtgccacccagatccccctgcaggaccacagccctcatccccaactactgggagtgttggcaactgacagtttttggcaattgcccactgctgaggagagatgcctctttcaaggcctcgctgctttccctgggacagcagaagcttgtgacatctatgagaggggattaaaggccctaccctgttgccctcatttaggcaactggcagggtgccctgtggggtcacctgatgcctttcttgtgacttctccctctggcctgtcctgcttcctcacttctttgcacatcttgatcctgagagcattcccctgtacatttcctgcaggctgatttcctcttcagagtctacttctagggacctgtattagtctgctctggctgctatgacaaaattccacagattgggtggattgaacaacagaaatctgtttcctcatagttctggaggctgggacgtcccagatcaaggtgtcaacagggctgattttgcttaaggcctctctccatggctagcagatggccgccttcttgctgcattcccacttggtctctcctcttgtgcgcgcatccctgatgtctctttttatgtccaaagctcctcttataaggactccagtcagattgggttagggcccaccctaagcgtctcattttaacataatcaactctttaaaggccctgtatcctaatacagtcacattctgaggtactgggggttaggacttcaacatatacattttgtgggttgggggaagagcatagtgcagactgtaacaggacccaacatgcagcacctatggcacccttggggtgccttactttttcatcacttgttaatctggtagcctatgttagcaggtgcctttcaagtgggaaggggttttcttcccagttgcactaacagaacctttgattcagttcagcaaacatctgttgaatacgtactgcccacaaaccagagtcaagagttgggaaagaacaagatagtatggtttttatttgttcattccaagaacaagaggaggaatgagcaagacatgttgtaactcttgtgatgtctacaccccagtgaaaaaaagagactcctaaacaggtgtctcctgcagcactggggtgtgtttcagtgctgcaggaccctgaggagggtgggtgatggctcaggcaggaggatatgggtggattctaagacaggaggattctaggcaagactcactttgaaggatgcatagggactggaaaagcagagggaagggaagtgaacagaagagttttccaaatccctttcttattaaagtagggataataattagacaagatcaagcatgttccaggtagtatggccatagacagggataataattaaaagcctccgagggttgttttgaggataaatgagacagtatatccaaaaaacttagcaaactatcaaaccctttacaagtatatgatgctatcatcagcagcagggagtggggggagaagaagagagcgatggtcaggatgggggcagtaagtgtgcctcacacgcctcaggcccctgacactgctttgcacagtgctttatgcccagcaggttcaataaatgccgattgagtaaatgaatgtcagaagcagccccaggcacctgatcaatacatacatgcaggcctcccgctgtgggctccaaatgggaagagggctcagttggttttgcagaatacagcatcaatatgacatatgcttcattaccttagaacaggaagatttgctgcgtggtggcattccatgtcaccccacatggaattaggcactttgcttttccccgaggctccagagtgatggtggttggtaggacatgatccatattcttctactaaagctggaagagtgggtgtgtcagacagggatgagagtgtagagattgggcagaccctcttccaccatcctctctgacccattgctggaggtgtgccctgttcattgattggacatggcaccacattcatcccaaatggcatcaattggtggttttcaatttgcagtagcaaagtacctggaagtcatgtgctttgtatgaaacgccttggaatgctgataagtttaattctattctgtaaaagaggaagacttttgttagctgaaaagccaactatattatccatgcattatgcttcagtcacaaccaaaactccggctgtcaagttcatcaactcctgatgcctcccaagtctgctcctgagcccctctggtcccttttctctgtgaatatgcgacaccagcctcccagagccccacgcagaaatcctagcattaccctcaactccttatctccctgcttgtcccatacccacttttgccccacacctatccaaggaccaaggaccatgaaattgacttgcagtttagctcctaattctgaacaggtctctccatcctcgtttccactatcttcgttcagatgaccacaatttctaacctaaattacagccaaaacatcactctcctctgagctctcttctacaccatacactttctagaacaccagtccaatagggtcatgtctctgggcccaacctctcagggactctccagctgacaagacacaatctggcctgtaaatgtccctgcagtttaattatcctcaacattactgccataccctacatttttggccaaacagaattgtttgctgtttgatgatttgataccatgtcatattgtctgtctggaacatccccccacccctctcatttagcaggctaagtcctccttctacttcatcccagatgatacccacttcaggaagtctttcccttctgtgggatgagatgcccattctgcagggctttcactccctgcatcctgccaaacctcatcatctcttacctggattcctgctacagcctcccagttggtgtcccgcttccactctgggcccctcctctccgttctccacagtgctgtcagaatcacctattcaaaaggcgaatccgatcatgtggttcctgctgcccttaggatcatgtataaactcctagcatgacttttaaggccctctatgatcttgcctattgcaacctccccagactcaacccttgccaggtccctctgcatcagctatccagaatctctttgaggccctccacctgctgtctacctctctacctctgtgctttgcatatactgcttccaatgtctagaccttctgctgactcctagttctttacctggctaattcctacacatccttcagttgtctgtctgttgaacatcacttcctctagaaagccttccctgaatacctgaactaggttatgtatctctctccctgttccatttcctactccctatcacccttagatgtaattgcttgattaattggctgttgtttctcctagaatgtgagctacagaaccatgtatattttgttcctgcctatatttctagaactatatagaacaatgcttaataaatatttatagaatcaagaatgaatgaatacccatttctgtttcgatgactagaaggtagcaagcctggttaactgaagtgtgtggtgcatgggtcgtgcttaagaagtgtttgttgaatgaataaataaatgatggatcagctctgttcaagctcatcttattatgcatttttaaaaaattgtgtttgctcatttgtcctaccagctgtaaaaacttgctagcaaaagagtggcagaaggcccaacatttttagaaaattcttagccgtaaaccttaaaatcccgagttggaaaattctcattattaaatgccaggagttggtcttcttcctggagacctctgcaagaggccctctcactgacaccttgtgtccatttttcctggccagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtaggtgggccccctgcttgctccagcactttctccagcagggccctgcactggacactggggactctagctccccactggcttttaccaatgagtttcctggtgcttccccaggtggtttttccttcactctgggcttactttttctccttacgaaatgggtagattgtttccttaataatcccaactaccatttgtaaagtgcttactaagtgctgggcctcacgtagggactttaaatatagcattttcctatataaccctcacagcagcttagaggctggcattattgccaccattttgcagttgataaaccaggtggtcagagatgttaagtaactgctgcagcatcacacggctggcaagtccaagctggaattctggcctcagagttagttccttaggtcatattggaaataggagtaaccggcaaggatccccaaggaggggcatgtttatgctcccaggcccctgaaaccttcactcccatctccccacttcagaaatgggtctgctgctttatgctccccatatcccctctccttcccacagcttatcctggggccctgtccaggacctgcaggtagaggctgccatggactgtgctgtagcccttttgttaggaagaattgtgtagtcactcatttcttggcccagctctgcacctcaaaatggggagtaggcagcacagtctagacactgtgcctggtgcattgctgacacagacccagtgaatactcttggtaaccctgggagtccatgctactcttctcactttacaaaataggaaacaggccgagcacggtggctcacgcctgtaatcctagcactttgggaggccgaggcaggcagatcacgaggccaggagttcaagaccagcctggccaacatggtgaaaccccatctctattaaaaatgcaacaattagccaggtgtggtggcatatgcctgtaatcccagatacttgggaggctgaggcaggagaattgcttgaatctgggaggcagaggttgcagtgagctgagatcataccattgcactccagcctgggcgacagagcaagactctgtctcaaaaaaaaaataaaattaaattaaaacaaaaacaaaataggaaatagaagctttggagaagttgcctttcttcacccaggtcacagggagaaatgctgatggagaaatcccaaagcagatattattctccaggaagacaccttcagagccagcgagcagatgtgggagcatttagggatcatcggtgggcacaagctggatcctgcccctttctctgcccagctttctaggatggcatgtccaggactcaagcaatctgggagtctaggtgcatgggaaggcatgggatcaggcatttaggaaagtgccggtgctctgcccactgaaccgttaccttccttccctgagcaggagaggaattgaccagtgcctctgaggccatccctgcctgagagggaaggggttgttgaaagaaaatgagaaagctttgtaggtttaaacaggggagaaatctagatgaggacgctcaggtgaggaggcgagactggtggaaagtggcacacctccctgtccctgggcccccaaggggctcgtgcgcttactgttctcaccaccaccccgggcccaagggagctctgatccatcaccccttggcttcctttactacagccaatgcaggctgcaacttccaaaatgacctgactggatattaaggaagaaccaaaaggaaacacaaatacaaaaccacataaaaatattcagttgacacaggccaggagtccaattccccctggatccatgtttatgagggsccctagactctgtccatgccttcttaattcttacgcttgatcccctccttcttccctcttccaaaggagggtcctaggtctcagaccatagggraaaatggtagagcaaacaaactctctgtgcctcagtttcctcatctggaaaatgcagataataatagtatcatacatcatagcattgttgaggatcgaattagttgatgtatgaaaagtgattagaatgatacatggctcacagtgagcactgtgtaaatgtcagccatggcgatgatgataaagatgaagatgacaatagacatccagcactgctccacatagggaggactctgtctttcctattcaccgcccccttgggataaagaggaaggagagagtactccctatgttcgccccaggcaagtaggtaccctcttcccagaggaacgtgtgttctacccagggctctgggcatggcttctgttagcttccagaccccctgtccctacccaggcacttgggtagggacaaatccatacatgggctatagggggttcccaggaatctgtaagtctttcgaacagatcctttaaacatgcttgttcaccttattttaaaggtgaagctgagtgtgtcaggggaggagggtgcagaagccattctcagagggcagggstcagacttccccacagcatctcatcaaactagagctgggcactgagccttgtttaatggacattatctcagcgatttgttgtccctagagaacgtcccaggtgacgtctcaccctgccctgatgtcctatcagcgcaccctcacaaccacgcactgtcccctctctaatacatgctcttgtcatccagattttttcctcctgttccatttccagcaatgccatgccaacatctgtctggcttaggactaagtgtgcgttatttatcttgctaagtgtatgaaaagcacgctggttttggagtgagaggaccaggttcaattcccagctccattcctttccatctagatgctattggaagagttcctgatgtccacatgtgccattccatgtcagcctcacaaccctttaaggagcggcttcctgtgtgcatttattttctagatgaggagattgagaagatgaacaaatatctcagagttattcattcactcagtcagcactgactgagcactccgaggtcttaccccaatccttgtgcaatgggctaaggcactgacctgctggggatgcagaggttgacagggcccctacagtgcagtctgctgccggtgacagtggagcagtccccataggaggtgtggggcataaggcccagcatgagggtgtcagggaagactttcagggagaaatgatgctttcggaaaaattatgcaggagagggagggagacacttgcattctcaagaaagccacccagcagggagagggagtagctgbctggaggtatggaggagaggtggttatgtcatttgcctctgaggcttactgtctgcattctaagatataagcatcaagtgtttggaacagtgcctgacacatggtaagtccttagtattattacagttattaggacttagctgagccagctcagggcctgtactgcaggtctcagctttatgtgagcaagagcattaaggaatgatgcctggatgcctgggggtgtgaagaaaagagccttgggttcgactagggaacctggggccactccttcctctgctactaaatcaccaagtgatcttgttctgttttcttctctgaccctccctagttttgtccacccttgaaataatcatctttccttttcacatttcatgcttaccaagtacttgtcacctaattatctcctctcttgataagctagatggtyccttccagggcagcttagtagagagcatgggatgtgatgtttcagattccagctctgctgcacacctgccaggtgaacttggccacgttacatggcctctctgggcttcagttccctcacctatgagtgggataagcaagcccttcttgtaaaagttttaagaacaatacatgagataaagtgcaatgcccacagtagatgtcttatggacagtggccaccaatgcattttcttgagtctttaagttagcagccctaactactctccaaggcagcttcccctgggaccacagaggaaatctctttgttattctgggctccagataggccagtgcaaggagagatttaagatgtccatgaaggcaggactgtgtctgaattgctcacaactgtgtccaggagcctaggatagaggctggaacacagtaatttgctcaataagtgtttgtaggaaagaagggatggcagaaaggagggaaggagaggaggaagacagggaggaaggccctgatatctcaacctagatccccagtggctcccaagtactggtcccaggaggggcttcaaagtgggatgtaaatcaggtagtgctggttttctgcccagtggtaccctaaaggcacactctgtttatgccaggaaagccctttacgtaccctcctgcctccacccgcaaaatgggcatccagctgttagctcctgccaacctggtcagcgcagcagcacagggagctgggagagaggccctggtacggggcccccatgtgagctcccaggaagacagtgccgcatgagaggcttgctgtgttctggggcaagctgctgtttctgccacacaggatgacagcagctgcattgcccatcattgagcttaaatgccagcatttggttggatgcctaggagcagaagagagggcagtgatcagaatgagcaaggttataattagccctgctgccaagctacacggtgacacgaagtctgccctactccgggttgcgggagggggctttcacgctcctctctgctcctattggtagaagccaaaggtgcatctccctgtcctgactccatcctcagggaactggggctgctccaagcttccagagcgctagggacagctgccgtgagtgtgtgcctgcgtaatctgccatgagtgtgtgcctgtgtgtgaatgggtgttacgtggggaggggggagggcggtgcgttcatgtgtgtgtgtgtgtgtgtgtgtgtgtgtgtgcctgtgtgcatgtgtgtatgagaaacgtgctcctgccaggagctagactcttcatttcccatcctaccaaggacacagctcatcacagcaacccctccgcctaccccagcccactcgggcgttcctcgagcactagcccagatctgccttctttcgttctctgggtataagaaggagcagaaatttcttctctgtttcatggcgagtcttcatcttcaccattattcttaacagatcattttggggccccgactctgccatgtactgggccaaccactgaagggcttgcctggctcagccagagcctgggccagagcaggactaggaactgagcccacaggccttggggaaatcactgcactttttggaggcttggtttcctcattggttctcagtttcctcacgggcattctaaaattttacctggaatctaatttacagaggtatggctgagtgataacacagggaagccagtgccattgggaagagtttaacgtgatacacaatgtccctagaaaccagatgctcagcccgccatgtggggtgcagggggaagcagcaggcttgggtcaagagtcagaatcaggggtgagggaaagcccaggctacagccctgttggggctcccacaggaagtgcaaggcagggcagattaagcaacctagggttggctagtctcaacatcctggcaggctttgggctacaggggtgatctctagttgtctctctggtacctgagataatttgggacaggggaaatattgtcttggtgtgtgtgtgaattagagaaacggggtggctggcttgcatttgagaggcatccttccaagtcagtcattttctgtcttcaggaattaactagctctgggacggtaagtctctctctgggtctgtgaggctcccaagtgccagaatatcaggatcagagaagatagaaaaatatagtcaatataggtgtctctgtgatgaatgggtgccaaatacacaaatacagaatctaagaaaacacatggggttaacaaaggttgcagagattaaggtttaaagttgaggccctccgtataagttggctgtctttcttctagcacagtaattggcaataagtggtcttatgtatctgggagaagataagtgagggscaagggcctcagacacccatgggcatcactccccacaccccagctggatgcccacaagacttgcagctgcctcctgagtctgtggcctcatcggctccaggagtaccaggctacaggacctaagctaatctcccactcctgctgtccatccattatgttgctttgtccccaggtggtaggtgagtggaaattcagcaggattcactgtgacatcttcgtcactctggacgtcatgatgtgcacggcgagcatcctgaacttgtgtgccatcagcatcgacaggtgagcccagccagggtggaagaggttgctctggccactcaccattctggctgccccagcttttctcaaagccaggctgtgtctcgtgtctgtgatgctgtgcagctggctgtgtgcatgggggtccatgtgtttgtgtgtatgggtgtgcatgtgtgtatgtttggctaggagagcacacactccctaaagagaagccatctatggacagtgatgctgccaagcatactcagacagtgactggtacaaaaggggaaagattgtgattctgacggaagccctagctctagcacatcttactcatgtgaccttgggcagatgatggacgttttcagagctctcagtttcattttctagaaacctacattgatgacacctgtggcctggagctgcagtatctggttatgcattttatgtccttggcacatggccaagggtcccaggccatgcacaccttgctaagctgcatccttggagccttccactaatttgcacagatatgaccctacctccctcacaccattcttggaagatgaaataagttaatgratgagaagggctgggtaaaaaaaaaaatgtgatgaaggattgtagacaattgccattgttatttcctgctaactaaggcttaaatcttgctctgaggggcctggctaacaatttatcaacaaacatttccaccttatgggctcaacacaagtgcagtgggtacagtttcaaaagtgcaaagatgcaggccatggttcttcgaaaagcctgaaatccaggtgggsccggttagaataatagatatgagctaagagtaaacaatagctcaccagtgattaagtgtttgggctttgccaactgtcctcgccatctgagtttggggacaggagggagggttgagttagcatctcaggcaagcttcatagaggtgtgtcaactgtttagccccaaggagtgagggtgtctctggtgtgtgcatattgtggagtgaggggtccctgggcctgcaccccagattcagggtcccccgcccttgcaggtacacagctgtggccatgcccatgctgtacaatacgcgctacagctccaagcgccgggtcaccgtcatgatctccatcgtctgggtcctgtccttcaccatctcctgcccactcctcttcggactcaataacgcaggtacattctgcttttgtttgcctgaggctcagctggccctgggcccctgctccctcgaagggccctgagggagcagagtccctggcacagatatgggtggaggcccaatggaggcctatcactaccctggactgagtccaggctacgtgtcctgggccaagccccactcaagagttgttctaggttggcagagaggaaacagtggcccaggacacaacggagttggatgaggagtggggataatggttcaggggtatgtctgggaattttcaactttggttattaagttgaagaggcaagacgtgcttcacaaactgcatgtgtgtatatgtgcacatgaacatgggatggcggaggctactgggcttccatccactcctgacaatagtatttttgtaatgaagctgcagccctcagggcctgctaaacccaggtcaggccttagccacctggagcaggaaagctagaagtgaataggaggtaagctgaccacatgcccatttctgtgggaggcagagtcaggagtgtctcagacagaggcaacacaaagcccccgccagcgtggaacttcctctcatcagggaggcaagagcgatggcaggtgtgcacctctggagatcgtaacaacaaaagatacagtgaagccttgaaaatgtgtggttcggtatctattataagcatcacaggaattctaagtgggaataacgctaaacatacttggattaatgcacaaggccctgagatagaggacaggcgtctggtagacctagaagggcacgcaaacatatgaaacacataggaacacaagtgagttcaacagacagagccaagttatcttgctgcaaacattaaaaggtggccaacctctcccaatacacaggtcagactaaaaagatggtttactcttttaaaagttttcttgtgtcattctttctggatacatcggcttcacttgttatgcccagacatggcaaaactaatgaccaagtaatgagggaatagtaatggaaagacttgggagcagtccatcatcacagcttaactttttgctcacaaccgtgtttttaatactctggtatctgctgtgcgtttgtgtatatctaagatgaccaggcagccttaaacatctagttgcgttcatattctctgtaaaatcgctccttgttcctggaaggacatgaatgggctcttgtggaattatggccggtgggcccgctgactccctgcctgcccgggctctccctcccccagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggtctcaagacaccccccaaccaactccaagggtccccacctaaccattaccaagagggctcatcttatgctcaggtgggggcttgggaaacctcagcaagggttaggtctaggctaaaggaattcccaggagccgggcaagggcagattttgaaggcatggacctcagtggagcaatctgagtctccaggagagggaggcagggtccatgacactaaataacaaggggaagtctttgcttggagatctttgtgactgaaggcggcaactcttgcttggtacccccgtgggctcctctcttcctcttttctggtttctctgtctcactttagctccctctgactcctccatcctctttcctttaccttccgtactctctgtcctcccctccaaacacacatcacttttcctgacttcctctccactatgtctctcctgtgcttctctttcatttcccccctgatgtcttgtgaattctccccttcactcagtccttcacaaggaagacagtgtgtgggcacagaaggaacaagagctcttgggctagacgcatcaggttcagatcctgtcactgacactttttttgctgagtgaccttaggcaagtttcttaccttctatgagcctgtttcctcatctgttaaatgggaatcaaaataccagcctcacagggtggtcttgaggattccacgtgagaagggacgtgagtgtgcctagcacagtgccaggcccttagcaggtgctccataaaaaaccaggtccttgtgttcctcgtcatacccaccactttttgtctcattccagccttcccccttgccccaactgcctcctctggcccccacccttctgatctctgagcccttctgcccagtctgagttccatggactgctgcactttgggtttctgtcccccctccatccccaccactttgtgtgacccatgtgctggctcactccacagggcaactgtactcaccccgaggacatgaaactctgcaccgttatcatgaagtctaatgggagtttcccagtgaacaggcggagagtggtaagtgctcaggccaggacccagagccaggtctttctgcccctagggaagcccactggccatggttctgagacctcagaagctggccaatgggagaagcaccccagaaacccccaccttgcctcagctgaaggcagactcaccgtgcacacctccaagcaggcatgaagtgagacacctcggttctgcaaggcatggatgtgtacgagaaaatggttggccataccaacgtaataaaaatgataataatggctattcacatttctcaaacatctaccatatccctattatctcatcaaatcctcaccacgaccccgggaggtaagtctctttgatcgaaccgatcttcagttgacagaagaggaaacaggctcagaaagattaggcaactcacccgtctcaagagttggtgacactaagcccagacctgtgtgactctgaaatccacacctgtgttctttccactgacatgagctgccttatggatgggcaggttctggggtaggacgagcagagcagctgcggggactggtggcggassagtttgtgtacatagagccctcaggtgcggaagcmmagcagaccccagcctctgccaggtggtagctgtaccaacatgcaagcagcaggcattccatcctccagagggatggagaacagggccagagaacccacagagggccgcatacaaaatccaggtctggtgtcctgccttcacctgcactgcaagggcaggactctaagaagctgtttatgaggcaggtgccaaaacagagcctcagagtcagggccaaggcagcagcccagtcatgccacctaggcacatagtgaggctgcactttagaagttcagactaacacctccaaggcctcaaacaagagaacctatggaagaacccaggaggccatgagtggatccatgccagggctctctaggtcaccccagcagggaagatgtggggccccaggggtcagccttttggcacctagattagtctatccaggagaatatggagcccacgtgtgtacgcaggtgcaggtacccatgaagtgggagcactgggccccttgtttgacagggagaacaggggtgggcaccctctttgcagtcggaacatgagtttctggaggcagggccaaacatcatcagctcttgaccccagcagccactgtggcacctggcactttgctaacagtaagtgttgctcaggccatgagagacaagtccctggtattcagccctggcagaacagaagtggggtattgaggctgcatgaggattgccatgggaaaaaggacaggggcaatcctgcaggggctgccatgggtcctgggtcccatgcctcagtgacatccttgcctccctggcagggtgaccctgtggtgtttgcaggagtcttcagagggtgaaagggaggggccagtgagatgggtggctgatgcctgggaacttgtccggctttacccagagccctctgcctctggtgcaggaggctgcccggcgagcccaggagctggagatggagatgctctccagcaccagcccacccgagaggacccggtacagccccatcccacccagccaccaccagctgactctccccgacccgtcccaccayggtctccacagcactccygacagccccgccaaaccagagaagaatgggcatgccaaagaccaccccaagattgccaagatctttgagatccagaccatgcccaatggcaaarcccggacctccctcaagaccatgagccgtaggaagctctcccagcagaaggagaagaaagccactcagatgctcgccattgttctcggtgagtcggccctggctgctggccacagcccggtctgtgaaaggtcccattccctgccatgtccttcctaaccatggctgcaggatcatggggntagcatttcctcaggcacaggccaagcccattgacatacagacagccctattaggtagattctgtaattgtgctcactttacagatgggcaaacagagttttagagtggttataaaacctgaccaagaggacccatggtttgaactcaggcagtctgactccagagtctggaaatttgactagtctattatgctggcaccagaacctcatgggcctatgtgcccaggaaagtcactttctctctctaggctcctcatctgtagagtaggttacttctcagggctgtcatggcaatcagttggtgaaagcattttctaaactacaaagcactatccatatgtaagtgtcactattatggtttttattactatggctctttttgaggaattgggaaattcagttcacttgcactcaaacaagactccatgggaccagagctgtgaaaaataaatgagatgttggggagtggcccccaatctaaatgaaaagcatccctgtccataaagcaggatttgtcaatagaagtctagaaaaccacatagtaaaggtcaaaacccaaaaggtggactgatatgggtggctgggaaaaggagggtcccctgtgactctgtcttccaggaagcacctgaatcccctttgactgccggatgatcctggggcgtgagtggggcagctgttacctcccttcttttatgaatgagagcacttgatcccatgggactactcacgatcatgggactgggatatggtaggctaggattggagccaggccctctgctgcagtccataggggctcagctctgtggccctactcttcccagacaccattcagtctccagggatgcctcctgtgcctgtagggcattccatgtacacattgcttccaggctaaatatcacacctgtgcaggaggtcctggaaaaagtgcacctgagttaggtgagcagagcaggtaggtggctgccctgggtgagagggcatggttcaaggcagggactagcaagggtgtacccaccaagactggggaaattgggggaaggctagagcgagatcatctggggacctggatagaccggagttcagctctcagccttgctgctcaagagtgaatgaccttgggtatattgcagagctgttgtgagtgtttttcttatgtaccttatgaggatgctataagaactaagtggcatgggtctatagccctcaaagatgacctggaagtagagtagatatttccatttctactggctaacctgggtcagaatggtaaccttttggatttattgttaaaccatgtacccttttctaggaggtgggaaagggacaaatgagggaagtgaggctgaaggagctgaagggatacttcactgcaaatgggtgtcagaggcaggtctaggatccaagaccgggactcctcctacagtgcttctgtggctacagcccaccgtcttggcatacgagccagggcgcactgggtgtgggtgttcccagccgtgcctccccggctctggggaccagcctgaccatgccctctcccccaggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccrgccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccagscaacttcagtcctgggagacccatgtaaataccagactgcaggttggaccccagagattcccaagscaaaaaccttagctccctcccrcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtagaaggcagcagtgccacttctgtgcttggcattcaagtataggaagacccctgtgtctgcagggtctaacccaagggaagccaggttgcccccactgntccacctcccctgttgcagctcctgcttcctctgaaggactcatcctttgccctcttacccaccagggcagagaaggctctgtggaaaaggtggccttggatgcactggcattgcctgtgtctgcctatgtcccttgncctgtcttctgtcccatgtcaggatccccttcctctagggcaggctgggagaagcagggaaggccctgaccactgcggcctggacagttctccctcctctcagcttccagggcggtcccaagctccaagccttccgggggaaaaacttggtactgccccaacaacagaaacttggctttctacaaatgaagcgtaaatcancccagtgagggaggaatattcttaccaccttgagaataaccgcagtgatgacaaacaaggtgccagcacccacgggctcaggcgctggggagctgtcagggcgtaatttgcatgctaaatacaatatttttagcaccaaagtttggagcacttaacttgccctgaacagttaattatggactttgatcttctccttaaacctaaaggtagcactaagccctgggagaggctcctgtccccaggagcaccctgattctggaaagtgagcaaaacaggcccctagtctaactcggactgggtcataacaccaaggacccagtgaccatctcctctggaaagcatcaggtccccaaggggtctagaagccccagggacccaacccatccccattgnacacataccatgctcaatgtctgtgaaagatcttggcctggatggacgcttaaaagtatatcccacaattaggaatcttatgagggtatacaggcttatcagatgtgangattggaagagatgacaaagagaagcagaggaaagaagaaggaagggagggagggagagagggagggacggaagggagacccagagcagagtgagaagagcattgacagggagcagaggggaagagggcagngcaggggcggnaggcggtgcaggggaaagttgcccacagttgtcacgaggcttcatgtctttctgccagacagcagattgacagctagagtgggcaggggagggctgggctccaccctctccccccctcagcacttcaggggcaaagaaatgggaggagtaggacccgacacgacacgggaacacaaggtggaagggggtggcccaggctctgactctctccagagaggtcctgacgatggtctcttgctcttgacgacaggatggaaaggaagcctccagttttcactcctctttgcctttccctaggtttctgtctgtgnctgtgccgctctgtagagtgccttttaatcaaaggatcattcatttgcctttacagcaggtagagtctgcacccttgtcccctgccctgccctttcctagagcacagcccaggatcaccacctaagggccaggcacagtggggcactttgcatatgtcgtcttagatgggaatggaaatcacacagtcacaaaggagcagacccagaggtaactgcaagtcttcaaggcttcaggccctcgttcttcctcctgggtcctgaaggactctcaggtggccctgggctggggaaagttcctgggaattagaagacgagttgtctagcagactaagaagttgcattgcttcgtccacccatctgctgtcttccttagggaatatttattgagcaattcttgtgctccaggccctgggtgaggtgctgggattaaaacagtcaacaatggtcaccggccctgccttcgtgagtccagtctagtgggatcacaacaaagtgagactctgcttcccccaaggcagagcccagggacggcagacacagcacaggcacatgctgcgcacttccaaggcacctttctcctagatgacgtgtgacacagcagccctggtgtgccttggccttgaacaattaggtagggcaccagtaggggcaggaagactgacagcatggaccactacttcctgcgtggctggggcaggaggccctgaaagaagcagctacagatcctgctttccaggtggtctcaccacaggcacagccagttgcctgcaactaagaacactgaagcccggcctgctttgggggcatttccagcatcccctcctatctggaatctcttcccaaaacatccctgtccccagggactgcatagctcctttcacctggtgggccagccctctctgtgtgccactggctccctgccntcctgggtcccaggacctgggctgagtgtgcaactttagcttccatctgcacagggccctctctgcgggctttgccctccctgatggcatgttcttctcctaacccactggagtgaggggcattcttatcgtctccacttacagaagagaagcctctcatacagaaagggtagtttccctttcaggctaaatcttctgattttacctcttatagtgtccattccagccctggcttctgaccagcgtgagtgcagctgaaagaggctaggatggtcagaagattttttttaaaagagaaagaatacttgttttggaccagacctgtagggtaacagcctggggtgcatgtaaacagaacttgggcctctccagggagcaagtgggtaggtggtgggggcagagcagttattagtggggcttcaggagtgagagggccagtctgggcatcggtgtgccagagggtcctctcctgccaaccaggtggacctggctctgctacaytagtttytgggggtgcaggggaagcaggaagctggtggggggagagtggaccatctttggagtagggagacctatcctggctttgtggggttagaggggacgtgggaagggggtacctggtaggaacaccttcctgcttcctctctcatcacagttctgctacttgscaaataaggcctcattggagcagaggcccagagaggcgcaggagagggaatggcaaggagggatgtggagagacctttcactcacagctggagccagagacaaccccaagttcataggaagtgctgtgtgaccgctccacttgcatgtggctttctcccactttcacatctggcttctgacacccagagctgcgtgtgaggccagggagagggggccacctccctggcaccccaggnccaggggtgtcagagctgcacaggaagtgaaggaatcctatcaggaaggggtgaggtggtcccattccaggaatgggagggcaggcctgggccaccgactgtgcagagacaaagctggccatccataccttctggattttcctgatcaggcccaactccaaacactctgtcctgttncccccaggagtgtgtaagttgggagggtcatttgggggccaggctctgtnctgcgtccagggcaggggaaagcatgggaggggcaagagaggcaggacactcccctggagaagacaagtcccttgttggttttctcccagggctgacagcacagacctgacccaggctctgggtagaggaggcatgactggctagtggggctagcctttgccctataactgacctgatccgtaggtgtttcccaggggncctcggtgtctcagcctacaccaaggcactgaaggagagtctccctttcctttcctgaatgtatcggcaaagaaaaatgggcagaggcttctaagagcatccttgcaaaaggctcccaagggatgggctgtctatgtcttattgcaagagaaacattcctctgatttaggaggaactgcaagaaatgaatgtgttgagtgcttattaactgccttgccctctgactggcagggcatcaggtttaattcttacaatgacccagagataaacattattgctgctagatgataccatcaagctaagatagttttagcaacctgagatctncaactanaaatggctcagcgaagacatgcacccaggtctgcctgcccgagtccactagacccagccacccgcagtttccctcnccgacctcagcccagcctggttcctgccctctgtccctaacattaccttacaccaaaaacccctgctgcctgtaatccgattcagcaagcagggaggaagcaccctctgagctccagacaccagggcactaagagaggactggatggaacacagaaacaagtagggcgtggtgatgtggcatgcaatgagcaaggaaggtggacccaagagaagctctgtcaacctcactgcactgggcatcgggacagaatgcccctgaggaaggagtgagcatctccagggggcaagcagagacgtcttcgtgggcaggaacaggcaagttggatctggaagggcagatcggatcccggcaggcaccggtggaagaagaggacacagcattagcaggaagaagggcctggggttggtgggtgctgcagcaatgctggctgggtttcagagtgggctnggagccctaactcactggaggccctcacttattggtgagatgtgttctataggtaagcacagggggnatcaggaaaggaatgttcctgaacacaagatcaccttggagctgycatctgtgtgttcaggaagtcctggggntccctccttcctctcctwcttgccacaggcctggagccctgtttcctccccttctcagacctgcccattatcattctcttcctggggctcagtgncctcaggttggctttggagagggacacggtctccccagtctcctcatttcccgtaggccttttcagccgngaggagttttctgctgatccctgcagtcctgcctcccttcaagacagtcgcggcagnctgtttaggcctctgccctacactttgntgtggagacagnttgmcaagnctccctgytcctttaggattcccagcctcataactggagggttccaccaacagcttccatctcccagcacaagaagggatccaggtggaacaatctcagctatctgcatctctggggatggctaggctaggggatgccatctcccaggacccaggaatgtcctccttgaggtgggctggattggatcaagcacagtttaggatgggaaaggaggctgtcatatgagtaaagggtaatgaaggggttacctgagtaacactggtgagaaaggacaggaacagaaagctggaagaggagagaagtgatggtgagtaccaatgtgtgctggagataggcgtgatctaattgcatccccaagataacaaatctgttataaatacagatgccgaaatctggaaagtctgggtaaagtccacagctagcaaaaggcagaattgggatgccaagccaggtgtgccttgctctgaagccccatagggcagggtccccatctcccaccactgtcccccatttcctcatcctgcacacagatgaagaggtctttaggatgcatggngtgttctgattgnatcctccttctgctgggaaactgacaggaggtcccaactgtccctcatcctgcatgggagntcccaaatcagaatgaacattaaggtctctccattgaatacgcacctcccacacctgcacctcaacagnactgtccctccttcagntgggcactctctgctccccatagcagntgtgtntntttcacattccccagccaatcctctccccttntatccagtccaggctttgctcctcttntgctctgtccttgtcctcattcaacatatgcatcttccaggaaaatctttcctgatcgaccccacgcaacatggcaactgcctggcggaggcaagacaggtatagactgtgctgtccactgtccccatgtggctagatacccacatttaatttattgaaataaaaaaacttgattccttggttacactagccacatttcaagtgctcaggagcatctgtggctggcggctatcctactgagcagtgtggatgtagaaggcttccatcctcccagtgctgatccagaatggctcctaagttaggagtttggataccttcaactaaaatagggtataaagggtgaagtcaggttctccctccaagtggagatgcccagtaagaaattggggagctctttccccccgatccccaatctcccctaaatagggcttgaacttctactggagaccctctccttgggaatttcaagggcctcattcaaacccaattttaaacatccctagggaagctctagaaggcagctgtgttgaaatgggactcaaggtggataccgtgtgtgtttgtgtgtgtgtctgtgtctggctaggagtggcttgatagggcagaagaagacactggtaggcaatgaagggccatgttgtccagggctggcctgaagtcacctctaagagttatctcttagaggaaggaagtggaggaagcaggggtgtgagaatctctgcctagactcaggatcaccagagctgaaatgattcccagggacagtcctgtccacatctccacagcatccctgccattcattggacactaggtgcaaaaggttcatctactttttctaactcttttcttcaaggccttttctcagttctcagctctttccaataggatcagccagccactcttctccctctttggttccctcctgatcactcccactgatggatctgagcacccttggctggaagtatgtagccccagagccttctctggctctcatagagtcctagatttggagttagatctggaagggcagataggatcctggcagacactggtgggagacttcagagtgcaaagagaagcaaacagccctgtgcctcctccacttccctcctcttgctcccgcatgttctgggaaagcttagaactcctaggggaatctttaactacaagttttgcctaaggcttagtctcagttgagactaaagagatttgagcctgggcagctagcaggrggagtagggaagatggaacagtcaggtggagtgtcccctggaaacccagttcgtcagggagtccaaatgggaagaggaaaaaggacttctggacaacccaagctgctgggccaactgatgtctgcaaggttatggctaaggaagggcacagagagaaaccatgggatgctccagtgcttctcagccctgggtgctcttcagaatcacttgggaattgttaatgatgctcacaccaaggtcctaccctggaggttctggtttaattggttgagggtagggccaggcgttggtatattttaaaagccccttgcccaggagattttaacgtgcaggctcggtttataaccatggctctaggacacactccagcttcctgggctcacatctgactttccatggtggccataagctatggcaaggtggtggtaaagggcccagggcccaacaggctggagctgtggtccagaggccaaaggatggaggacacaaagctgctagctctactgaaagggacttgttaattcaacaggattgcacgtgggcaacacattttcntctgccccacctctctcaccccatgctgatgccaggctgctagctgctgtccccatgcccagactggagtgcccagcccaggagaatgacacgcagaggtgggcctgccaatcagcttggctgagagctggccaatagttgggacagatgattccttttctctgacaattgcaaaacatccttgagtgggaggagctgcttctactatgctgaccagcagtgtagggagacagaaggagcaggataggaccccaggaaactcatcctgtccctactccctccaaattgttggctgctctcctggaagctgaactagggccctggtcacataaagttagtgtgaacagctccaagccacaaccagcaggacaggacaggacagctgatgggtggcctgggtgtggtcacaaaagtgagccctgcaagctccatactgagtccagacaacccacccctcaccatcatcaccccctccaactccctctgctgcctctgcctcctgtctttatgtgaactcacacacacatatgaacacacatataccctacattagacaaatcgcatacacacacaagcacaccatactaacgctgctggcctctatatgcaacttggttccacaggccaatccattctctaaacaggagcctcaggagcctcagctttcttgacttcaggagtttgaaatctggctgtaaaactggacacgatgtaggatgaagggtggtgctggggaccagggacctcacatctcacatagggtgacttatccacagttggagtgcacccttcctatatcctcaaaatgccatgagggagacccaggcattgggaccaggggcactggattaggtgtggtcagtgtggcaacagatggctggctgggactttagtgagcagaaacctcctttgaacttttagcttctgccccttgagcaaagcacatgtgtctggctctccccgccccagcctgaggtaaggagccaagagcccaagacgtgagatgggaggatcctgcttccctcaccgctgtgcctccctccatctccagagggggtctgctaggatgcaagggcccctgtgaactcagaagcgagtggcaaaaggtggtaggtgtcttggattggagagggtattcatttggggagcttgcatccccataaaggaggaggaggaggcctggggtctgttggaagtgactggaggatgtgttgaaggcagctctccaagccagagccccttcctcagctacagccacagtgatacctctccccatgcccccatcacccaggttcctgtctcctctcaaggtggaaggggcaggtgaagactgcagacagggaagatgccctgccagaagcccagctgcactttcaccaattcntactccatccaggcggagaggccccaagtagtctaaatttctttctttctttctttttnatatggagtctcgctctgttgcccaggctggagtgcagtggtgcgatctcggctcactgcaacctctgcctcctgggttcaaggaattctcctgcctcagcctccctggtagttgggattacaggcacgtgccaccatacccagctaaattttgtatttttagcagagacagggttttgccatgttggccaggctggcctcaaactcttgatctcaggtgatctgcctgcctcagcctcccaaagtgctgggattacagacgtgagccaccacggctggccaagttgtctaaatttccatctcggctcctggcttagaaccacccagagtggccactgacggctccttgccctctaggaaggacatgatgccctgctttcggctgcggagggccagttgcaggggtgtgcagctcactccatcctggacgtccagctgggcgcctgcctcgaccagcactttgaggatggctgtgttgcccttgagggcggccaggtgggcgggtgtccagcccaccttgttgcgggcgtggacatttgcgtgatgttctakgaggttgatgacactcaggaaggtgctcctctggaccgccaggtggaggggtgtccagcctgactgctctgcagcattggggtcagccccacactgcagcagtgctgacaccaccgcctcctccccgtggcgtgcagctaggtgcaggggagtccagttcacagctccaagagcacccatgtttgcgtggctctctgccagcagatggatgatctccargtggcccttgtangctgctagatgcaggggtgtccaaccctggtgggtgggcagctcaagctggctccgtacctgagcacatcttgcagatcaggtatttgcccctggcagctgcagtgtgcagtgggccatagccgctctggtcaagggcatcagggaccgctccactcttcagcaggtgttggatggccctcactttgccccgctctactgccaggtgcagtggtgttctcaggtttctctgctgancatccaactcagccccctggctggtcagcatcttgaccaggctaacatggccaaagtaggcggccacatggaggggggtcttgccctcagcctcacgcaggttggggtcagcctgacgggagaccagaagccgtgccacattctcaaagttattctgtgcagccagntgaagangggtccacccttcacgttcctgggcatccacacaggccccgtggtccaagaacangcgcgcantgcggtcatccccattctgggctgcaaantgcantggggcc

SEQ ID NO: 7 - DNA, 3695 bp, Homo sapiens
gcacgcgtgtcgacggcccgggctggtatttatgtgcccctttcctcttaaagtgaaagcttgtatttgttctgagccccagagcagcttctaagccccactatttctgggctcaagcaggtgaacaaagaaaaactattgctgtgcggtgattgccaggcagcctcttcccagctgcccaggacctgtcgcccttgacatctccctggctgtccctatacaccaccagcaccaagcttcgcaggtgcctgctgggtttctggcttgcatctcttcctgcggccaccagctgacgcctgcattcacattctaaggaacccgtctgtcccaaccaccacgacaacccccttcccccctactcttacctgcagcccacgcaggtcacagccattagatccttcccctgaagcgctgcttccctcctgccgcactcagggctccctcctgccttccatgcccaggacagggcctcccggcctctccaggtctctggagcagagaggttgtgccttcctcatgcggtcaggctcaggctgggaaggagcggcccagaggcggggggaggcacgggtgcggggctgcctcttccccggtagtggacgctgaagcctgtccacctgaattggaggcggggcggggcagtcgactgaccgccaggcaggggagcagggcgcgggctcagagggaggaagtgggaggccgaggaaggacaaggtgcttgtggcgatgagtatcagaaggcagagcggagctgctcaagagaggctggctctgtggaggtgaggacctgcaggtgggcctggcctctctgtgggatggtgcccagggcaggaggaagagaaacacctgtttagagagagggggagggtctgggccagttgtgtgcggactctgccatggaggcaggggaaccagggcggggccgtctgtgtaattctaatgtcatttgctaatggtggttgcccgcagctggggaggggagaggggaggagatggaccacccggccagagagaggaggggtcgaaggcccccttggaggaagggcccagcgcacagggcctgcaaggagttggtcccttccccaccacagctgggcagtgcccactgagatcatcctccctacttcacaaacagagacacaaaggccagacagcttgaggttaaggagctcggtcaagcccacaccacagtacagggtgggggccgggatttgacccaggtccacagggtgcccactctctaggcagcacctgcacagaacagggcagtgccatagggaggggctgcgcgctgggcaggaatgcatcttcggctagaaagagtctagaaggaagatcagaagtggcgagactgcacaattaagaagtgctaagaagtcacactggacagacctggtcttggtagccaagcgctgaagtcacccctgcagccgtcctccgctttggcgcctcttcccagcgtccctgcccccctcctttggccctcctggaaaggacactttgcgttttctgttgcccttgcctatacagcacaaacatgctcatttaagaagtggaacgtgggaggcagcagacaactgtgttcatctgaaaggaggaggccccactcccgtgcagcgctgccctgggggtgaaattcccaagcttgttgggattctcccgcctcggttgccgctctgaatgccagcacctaacccctaatgtccctactgcagcctcccagcatcccccctgcaacctcccagcaactccctgtacccctcctaggatcgctcctgcatcccccattatcccccccttcactcctcgcggcatcccccctgcaccccccagcatcccccctgcagcccccccagcatctcccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctctagacgctcagcccaaccggccgcacagttttcaggggtcagttcctccaagtacaaggggcggtggcttctctggagctgcaaacttgtcactgctatttcctttcggtcttctacttcctatcgttcctggcctcctcttggggagaggtagagccctctcctttccgcctcagggacaacccaaagcaagtactgcatgtgccctttttaaagttttaaataattttagcaaaaaggatattaacattaaatcaatttttaaactttttgaaaaaattatcaaaactacatgcacatggttcaaaacaataggctcctgctgggccctttcagataattcaaattgtcaccaggttggagtgcagtggttcgatcacggctcactgcagcctcgactcccgggctcagctgatcctccacctcagcctcctgagtagctgggaacacaagcgcgagcaaccacgcccggctaattaaaaaaaattttttttctagagatggggtcttgctgtgttgcccaggctggtcttgaattcctgggctcaagcaatcctcccgcctcagcctcccaaagcactgtgctcctttttgacgcagctttgaactgtagctggttaacaaaatgagaaccagttcttcattccttcattgtggaagtctttattgtgagactctggggacggagaggaattagacaagggcctctaagctgagctcacatcccagccggtgagtcagataaacgcatgggtatcgagtactgctaggtcccaggaagaaagagagagcagctttcgggatggggacgatggggaggtgtccgaggtcaagagaaagcggcacgagcagacccctgtgtgccgtcctgtgggcgcggggcggcaggggaggcgcacacctgctcctttgtgcagcctcccccctcccgcaaagttaaagagcaggaaagtcaggattcctcgctcggccctgccctgccggctgctccgcgctccgctcctccctgcgagcgtgtgtgtgtgtcgggggtccctcccctcctggctctggggttcgggcgcgcaccccgccccgtagcgcggcccctccctggcgagcgcaaccccatccagcgggagcgcggagccgcggccgcggggaagcattaagtttattcgcctcaaagtgacgcaaaaattcttcaagagctctttggcggcggctatctagagatcagaccatgtgagggcccgcgggtacaaatacggccgcgccggcgcccctccgcacagccagcgccgccgggtgcctcgagggcgcgaggccagcccgcctgcccagcccgggaccagcctccccgcgcagcctggcaggtgggtccgtttttcctctccgcctcgaacccacgtttctttccagaccttcttccccgcctcggggagggggatagagccgctgcgccccaccgccctgcgaggaggcgaggaggtgcatgcgccccagcggtgggcgccggatcc

SEQ ID NO: 8 - DNA, 5230 bp, Homo sapiens
aagcttagaagagggcattctcagaaaccaaccttcttaacaactcacacttctctattcctgggccagcttgttttcagccattgttatgtgcaagtcttgatagaaaaagccagccagggctctgcagaatcagaaaaaataggaagagagtctggggctccacaggcaggagcttgcaggcagaaagggcccgggcacatggcatcttctcctggcagacccgattagtcaccgagaggagttttcctctacctggcagaaggcagaggagtaagttaagaaaaggagatgtgtgtctcctctgagtggctgtaactttgtgtttgcaccccagattctatgatgagggtgggggttcagtaggagaaccagaaagcacagctggatagagagagacagcctgtggggagaggctgcccggtcatgagttgtctgagggatgctctgggttttcttgcactggggccatttcagcctgttgatgtaaaattttttaattaaaaaattaagaagaactaggggatatttcaggaaacaagacctctgtaatggctcagaacacaaatagaaacagcgtattactcttccaggaagctgcttaccatagttgccaggaattcaggactggatgccttcggggctgaacccctgaggactcctgagaacacacgttgtcctcaaaaatctacctacctgcacacattttctaccctctctgcaggacgcctcaaggaagaaagtaaacaaaatccagaacaaatcatttggcccctgaggcaggagggtaactgctcactgctgctgctaaatgtctatttcctctccccaaagtattgcttctgagatggaccgcatttcccttccgtagaccctctgggcctgggtagagaggccagggaacgcttgggtgatgcaattggagcttctatttaagtgagaggaaagtggcagtacagaaaatagaagaggttcttgcagaaaattgtggggccaggccaggcatggtgtctcacacctgtgatctcagctctttgggaggccaaggcgggtggatcatgtgaggtcaggagttcaagaccatcctggccaacatagtgaaaccccatctctactaaaaatataaaaattagccgggcatggtggagcacgcctgtagtcccagctactcggaggctgaggcacaagaatcgcttgaacccgggaggcagaggttgcagtgagccaaaattgtgccactgcacaccagcctgggcaacagagcaagactgtttcaaaaaaaaaaaaacaaaactgtggggccagtgggaggaagagcaagggaaggagcagctctgtcaactcggtgccctccagccgcacagggaaaagctcttcctccctgccatttggccttcaagttcgtcagttgtttaacgtacaaaacatgtcattgatatagccggaggaagtacagagcctttttaaacaacacaacccactgactaccaagttcaggtttaatttagctgaatatttaaatatgttgacatctcatcatgtaataaaataataatatcccttggcctggtaaatggttacctatggtgggtgagaggatgtgtggttattttaaaagcctatcaagcctagttgagggcccaaggaagggggaagtggggggaggccaaggccagcagtagcataaatggtgagcagggtgaggttatggagatcgctcttgtcagaattcatatccttctgagtggtgtttgcattcttgagcctggggtgggtggtggagggcccaacaagtgtggtacagcccaagacgtctacctccccttacacacacacagcccctagctacatgaagctgatttatctcttgttgctgtagatatttttggcaacaatcgatccctttaaaatttttattttccactttacagaaaaaatgcagcaaacgccaaaccctagtgactgacattgcctggtgttcatgtctaatccttgctcatgtttatagccatagtcattcttgtgaagctactattttgtgtcctgatttttcacttactatgtttttgtcttcacatgtgtaggtagctaagttgcagtcaccaaaaaccccaaataggtaggagacatccatgcgccatttctattagccagttcctagccatcctcctaactgttggatctggagggttagcctcagatcattgcagttataaacagtccatccctggctatcttttctagaagggtccagtgtcatctcagctaggcagaaacgtatttcaactcccaaactgtttcaagagtttgaaaagctcaggcccagaagaaacagagtgagcagttgaacagtcttacaactgctgcttacaactttataacaggccataggtttaaagtccaaaagggctcaaatttctaccccacgtttgacgactcagacctcaagccagagctgagctgacttccctgcgcccaggccacggcggtgaaatgaaggcacagcagccccggccaggccctcccagtggaggcacagggggacggcgaaaccaaccaccccagcatcagtaacctgcacactcttctccctaggtctcctggaggcaaggcgaccttgcttgccctctattgcagaataacaaggggcttagccacaggagttgctggcaagtggaaagaagaacaaatggtgagcagcagtgccgtttaaggaccagtcgggcttgcaaatgcataatgggaacgcagaaaggccatgtgcagatagactggacacagccggaggcagctcaaatctgccccaaccttcctaggggctaaaagacgagtgacagaatctgtagctgatgagctggggtgcctcacgaagggtggcctttttacctctgtcctttgcttttttccttttaaaatcatagctgtagttgatataaaatataactttgggtgtgcagcttaaatgatggtgaggactgtcctgcctgctgaaaggcagaaacgcctttgcttgtcattgtttccccaacatagcatccaagacaaagggggtgctctgtgacgctttggagcctctctcacattcttctgagctgctggcaaaccacaagatgagagtccagtggctcaaagggaagcgagtgctttgctctccagtccagcctgtgcaaacctaggtgatttgctgtctatttgtgttgttttattgtttccttttgaaattacttgattggcatttgatgccctgtataggttgagaaatttagttgaatttatggactgccatgtagcaaataggatgtctcacatttaacagatgcatttgtaatcccgttaactaatgtagtttaagatgtatgatgagttaataccaaagatgagtaaaatgtctaacttttttctccttcctctgtgtgtctttccaacagagtcaatcccgacatatcaatcccgacgatagagagctcggaggtgatccacaaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggtcagtatcacaggctgcgagtagtggctgtgacccagggtgggctgtgacccggagtgggctgtgacccggggtgggctgtgacccgggtgggctgcgacctggggtgggctgtgacctgggatgggctgtgacccgggtgggctgtgacctggggtgggctgtgacccgggtgggctgtgacctggggtgggctgtgacccgggtgggctgtgacctgggatgggctgtaggtcctcttgagaggccagaagacagattatgtcttttcagtcttcactggcgtaagactaggaacatgatgacttagactttcgagctggtagaaaagtcaaatctcgccaggcgtggtggctcacacctgtaatcccagcactttgggaggctgaggcaggtggatcacctgaggtcaggagttcaagaccagcctgaccaacatggcaaaaccccgtctccactaaaaatataaaaattagctgggtatggtggtgggcgcctgtagtcccaggcacttgggaggctgaggcaggagaatcacttgaacctgggaggtggaggttgcagtgagccgagatggcaccactgcactccagcctaggcgacagagcgagactctgtctcaaaaaaaaaaaaaaaaaaaaaaagtcaaatctccagcaagccaaaagctgaagacagcgtgtctgaggctctccgggttgtgaagctgggtgaagccctcccaaacctgacctcacagatctccagcatcattagcgctccatttattgaagggcctccttctcatcctgcattctccaggctctgtcctcccccagccagaagtgagggaatggccgtcagccctgtgctgagtcccttcactcctgctggcaagttctgctgggaagagtccacgagggtaggcctgcgtaagggaaaatttggaggtggcttcagagtcccagatgcttctgatcctttggacctgtgtgaggtcagagaagggcatcagcgttaagtcctaaatcttaattaccagtctgggcagaatttaagctggattagatccacagcatgacattaataccaacctcaaaccacgcatggctggccctcctatctcctcctgggatgttagatttttgagccagaggccctatcttgttccttgtacccctaaagcctctcaagtagttaacttgtagattataaaaaatagtagctgactgtaggcatgaagatgggaacctgggggagagcagagcagtggggaaaagtgaatgggtttggggcctctggacttgttatcagcacaggaaaataacaagaagtgggtgtcagatgcgtcaagctt

SEQ ID NO: 9 - DNA, 1494 bp, Homo sapiens
cccccagggttaatggagggcacacagttttcaagatggaagccccacccttcctgcctcccaggaactggggtggccacgtcagacggagtagagtgtgaggacgcacttggtgtgtgtcaaaacctgattgacacataaggtcttgtgatgagaattgtaactgttgttgtggctgagttttcctctgtgacatctttgtaggacaggtcttgtcaaccacctcctcctctctcccctctgtctcaggtcccagcctcctcttcatcacgtatgcagaagcgatagccaacatgccagcgtccactttctttgccatcatcttctttctgatgttaatcacgctgggcttggacagcacggtgaggaaatgggaaggggaaatgggctgtgggaggatgaagaggaaaggaggaggactcagctggactcaattccctcccatccctggccatcccaggcagctttggctactcccttctggtgctccagacccccggctgactcctcggcatttaccaccctctggtacattcgagtcattaatgggataaagtgggtcaagcgccaggcgcttataaggcccaaggctcacatatgtaaactgttattaagcactgtcccttggcccaattatggcgcctacagccgggcagtctctcctgggtctctgctttgcaaggtcaagttcaggcctggaattattagtgtggagtgagtctcagctttgagttggcctttctggtcgaaacggagtctccccgagagacctcatatattgaagttctgtctacgtgtgctattttgtttcctggctctccttagtgatgggctttaaatgacagtgtaaaacagggaaatactctttgagatgcttacatggtccagcgagccggggaactggtgatgatcagcttgaccagttacttagcaaccttaccccttcctcctgtttactgtcctgaaggccacagccttgtcccaaagcctccaccgtgactgcttgcctgtacctctcagtttgcaggcttggagggggtgatcacggctgtgctggatgagttcccacacgtctgggccaagcgccgggagcggttcgtgctcgccgtggtcatcacctgcttctttggatccctggtcaccctgacttttgtgagtatcatgcccctcagctttgagttggcctcccctagggcagcagaacgctacaggaagctgttcgtctccttccatctcacacagtgccctggctttgggaccaaaggaggggaggttgttactcccattaaaaggtttgtcatcaaggagaagaaaagttgtttcttgtgcatctacttgcagattccgtgcatagtatccaggggctgtcagcattttaccgcagatcccactagatttcaggcttcaaaaactatctggagaaaattaggatttgcttttccagcctatttccactctttagagctagctttcttgaggatactgccctgacgt

SEQ ID NO: 10 - DNA, 849 bp, Homo sapiens
gattttcttagtttacagatgtgtatagtttctgaatatccattttaattcatatttgagaatacataaattggaattcttttcctaaggaatgtcagtgagactattccaactcgctcttagatgttattaaagtgttatttaagctttgttttaatattaatgttgactatttttgcaagttttaaaaattacaaggatgtttataacattgtattttcttcccaatagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatagaattttattgctggaatctactagaaccttctaatccatgtgctgctgtggcatcaggaaaggaagatgtaagaagctaa

SEQ ID NO: 11 - DNA, 7282 bp, Homo sapiens
tcatctcccaaagtaactgggatacaaatgcctcccgccacgcccggctaatttttgtatttttagtagagacagggtttcaccatgttgccagctgtctcgaactcctgactttaggtgatccacctgcccttgcctcccaaagtattgagattacaggcatgagccactgtgccctgccgaggtggaagtttggagatgggccacaaactcctggagatagggccagctcagtccccctgagcacccacacacaccctcctgagagccaacagaaggagtgagggccccgaggcggatgacccgtgtgcctcaacccgaacggggagaggcggggtctgcagcagggtacgggcaggtgatcccccaaggaaagattttcctgtattgagagagaaggggccaagaggaggagcttgtcaaacaccacagcccctccccctcctctcagctccagggggtgccctggtgccagtgttcggctgatggagagagcggcaagcgggagagagagtgtgacccctgtgggcacatgacttcccttgctgcactgctgcacatagcagaggtgtggtgacgaccctgttttgtcccattgggggcgtttgctgttaggtctgcagaatcctcagttgctattggaaatggtgacatcactggcaggggcggagcttcagccatccttcaagttagggaggggcacgcacactccaggggtggagggggacaaagacagggtggtgtggaccagagggatgggtaaggctctggaaaagggggcgctgggagcgcattgcgagggggctggagagggagagaggagcggaagctgagggtgtgaaacggctggccccgaacacacctcgcggcgctccagtgattcctggtgtccgacctcagccccagtcagtgcgggtccagtttccaggctctcgcggaaggcctggctgagcacatgcggcagccacggtcaccctccctattcctcttagcccgaggaggggggtcccaagttacatggccacgcagatggggcctctccctcatttctgaaccttgtggggaggggaaccttgaagggagcgccccccagagccatggcttagggcctcccccacccctctggagctccagtctgcaagagtcaggagccgaaatatcgctgactgtgggtgacgactcttgcgcgcacacacacatacaagcgggcacgacgcgttcggtcctattaaaaggcacgcaagggtgcgggctgcacgcggtgacacggacccctctaacgtttccaaactgagctccctgcaggtccccgacagcacaggcccctgtcccaggacccctccaggcacgcgctcacacgcacacgcgcgctccccggctcacgcgcgctccgacacacacgctcacgcgaacgcaggcgcacgctctggcgcgggaggcgcccccttcgcctccgtgttgggaagcgggggcggcgggaggggcaggagacgttggccccgctcgcgtttctgcagctgctgcagtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtgccctcccccacctccgccacccacctcccctctcctccatcctgcaaccccacacccccagtttcattccatcctttccgtgcccccttcctccctgtaagacaccaccccagagtcagctggctgcttccgggaggcctcgtctcactaggaaccaaacaccagggtctgctggctcccctatcttggcctgagaccagtcacctgccaccttggctggtcctcagagggcccctggggctccaggccctgactggtgtgtgtagacgtggggctggagtgtgtcagtgtgggggtgggcattccgggtaagagagtagaagcgcctgtccagctacatgcccgccctgcagagctttaaacaggacggggcctggggccatctttgtttctgcttccaggttctcctgccctttctttcgtcccttccccctaccgatgggtccgcctgggaagagaaatggctcaggtgccacggcaggacgctttgtgggggtgggagtgggggtgcacacgcgagaggcatcagggcatgggagctgtcggcagccagcgctgcgggggaggacgtggctcctgggattttgcctgtcggagctgtccgcccctgggccgagcgcctgctgaattccaatgaggctgcaaggatctgcaatgcagccctttatgtaagaggcaagacagacatccagcctagcaccgctcacacgtgcctacctgatggacacaccacatctgtggacacacatgctcacactcacaccaaatgttacattagcacacactcatgcacctcagcatcacacaatcaatttcatatgctcatctgcacacatgcagatccattgacacctgctcatgtgccacacacggcttggcatgcattcccagaggcacgtgcaaacatgcacatttacacacatggttccagtcattcacacgcatgtacacgaacagacatgccagggcatgtgatgcacataaccataccctagcacacgcgtgaacacctgcatggtcacacacggacctacgggtctttgccaagcacctctgggtgcaggctggaagcaagagctgggggagggagaaccacttcaaacagctgcagctgcagggcccacaccagagttttctcagaaatcctccctccccacttcacaagccacccccgtgccccagcccaggacaccatgggatgggactggggggatgcatctgtagccagtggctgcagtcacatattccatctgggactggggagggacacggaaggtggactcaggaaatccaggaggggccattcctggggaattgcttcaactcacgcccatgttgctgtctgtctgtgggcatggcctctgcagcaaaggcaatgcctgcagctaccactcacggaacacacccccggccaggtactgtcctgccacgtggggccatgcagtgcacgcccccattcgccaaagctctaagaggcacaggcagacttggggacagacgcaggtccttgctgtgtgaaggtggtgtggacccccagctgcctgcctccctgccttgggaggctggggagagagggaggcatcagctccagggggctgagccgctggctttagatctgccccatggggccttggtcatgggcaggaaggctgggctgcacccccaatgcctcccttcccttccttgaggatgaggccagcactcaaagtaagggcttggtgttgttcagacagagcccgtcacaggccctgcccctggagacaccagcaaaagggatctcggcctctttggcagctcctagctgcttccccctgaagtccggtaccacccttcagagctgcccgccctgtctcgggatgtgggctggccccacccctggcccatcaggaaggacgggtgggttctgagaatcaaggccatcatgatgcaggaccagccatcctccccggtccaccttgggtgcgtcccgtgctccaggcccccaggacatcccaagggcagtccttcacctggcccttgagcacaacacctgcagggccctagtcagcagtgtgagaggagtgaggggaggtccgggtggggtctccctcccctgccctgtgggcatgtgtgcatctgggcctgggcatgtagcatgtacccgaatcatgcccccagccccccttagcctgctgggttcagcccctgctgcttccagatctcagcctctaacccagtgcctggctcacccctgactcaagctgatcatgtctcctgtgtccacaggtctacgccatcctagttagccatccacctacccccaacgaccacttcactcccacccctgtctcctacacagccggcttctaccgcatacccgtgctggggctgaccacccgcatgtccatctactcggacaaggtaagcctgactgccagaccaggccttccggccctcggccccagggcacagcctggccactccaggagcagcgggccgacccgctcacatggaactcacacaccacaaacagccacacagctcccccacattcatgcacgtccacacgctctcacgtgtccaactcacacatctgcaaacatgctcacatgcacactcatgtgctcttacacacacaatacacactctcttgcacatagagggctcacgtggagcccagcacgtgcccccagcccagagcaggccaaagggagggggcacacatcacacactcacacatcacacacacatcacacactcacacattcatacagcacccacacgctacactgctatgctcaccctccccacacatgaacactgacacacccatggattcgcacaaagtcaaacacactcactggcacaggcaccagtgacaccccctcaggacgagagggcccgtgggctaggagaagggatggctgggaggctttctagacaggtggactttgaaggggagtttggagagctgggggttgctccaggaggaaaggggtgtgcacgcagccagggtggtggggccagcctttcccactgcaggcatgggtggagagcaatgtctgtggttgcagctcagggtggggggcgctggcctgggggcttccagcctctagggctgagggcaccttggcttagcctcctgcagaccctcctggcccacaggctatgaggagggcttctgtccaatcctggaacatcagctggaagagaggagggtcatccagtcagttttgcaggaatctccaagccagagagccatgggggcttgctctaggtcacacagccctccgtctacccaggatgcaaactgggcactgagaggctgaccaacctggggccactggcagacagacctgcagggccacttggcaggggacatccagtttggtgccagcgctgaggagccagagggctgggctgtgcagccaggcttctgggtcccccaccttctccaaattcctcctgcccagagtccacagtccttggtaacactgccttaaagcacaggggtcgccccagccaggcccaggctcttctgggaggatggaaggccccagaggcaggaactgagacagaggctggaacagccaccttcctgaggctctgaaagccctggcgtgccccctcggcacccaaactgctcctcccaggtgacttcacctctggccatgggaaggtgggggtctcattcctgtgccctcaggcacgacctccttcgcctctctgggcaccagtttcctcacatgtgaaggagagaagatggccttacccaggaaagcagccagtggtcaaatgaaaggcgggggaaacggatgcccctggcccagagcaggccaaagggagggggcacagctcacagctgcaccctggccttaccacagtctctacactacaaccaacctgtgcccaaaacatgaactggcaaggccaggtcagagctagtccaagacctcaagcacagcctgccttgccaccacgtcaccaggtggatagacagaagcaggggacatttttgcaccccaaggcactgccccaggccacaaagagggagcaggtgaaaaataacctggaagcctcagaggaccacaagatcagcaagagtccacagggacactgaaggaaccagggcttacctggacagacacagagaactgaggcagaggggggcagagcctgctccactcccggccatgccacggcactccgtggcagcttgaggccaggaaaagcaagccagggcaagcaagcaccacgctctcgcctggggagatgaggcctttagccccaagagtgaattcttcttcatacatagagttgtttaaatttgggaggactctatgggcagccccagggggatcttcgaggcgctatgtgtcatcaagaatttcctgagctcagcttgtccaaaggtggtgggctgcaggggaagaggtgagctcaccccaggcacaattccacagaaacccacgtcccttagggtgctatggggccaacactaaacctcctccatttccgagattatatgtgggaggagaggccggggtgggagagaggttcccagggtctaaaaagtgtccccaggatggtggggacaggggtgggaaaaaggaggggtcccagtgtctagaaagtgtccccaggttggccgggcgtggtggctcacgcctgtaatcccagcactttgggaggccgaggcgggcggatcacgaggtcaggagatcgagaccatcctggccaacatggtgaaaccccatctccactaaaaatacaaaaaaattagccgggcgtggtggcgggcgcctgtagtctcagctacttgggaggctgaggcaggagaatggtgtgaacccaggaggcggagcctgcagtgagccgagattgcactccagcctgggtaacagtgcgagactgtttaaaaaaaaaaaaaagtgtccccagggtggtggggacaggggtgggagacaggagggggtcccagggtctagaaagtgtccccagaggggtggggacaggagtgagaggaagggggtcccagggtctagaaagtgtccccaggggtgtggggacaggagtgagaggaagggggtcccagggtccagaaagtgtccccaggggtgtggggaccggggtgagaggaagggggtccccagggtccagaaagtgtccccagaggggtggggacag

SEQ ID NO: 12 - DNA, 13440 bp, Homo sapiens
gagctccttggagtggaggccaaagtcccacagcagaggccgactggcccacccagcatgcccatctcccaggagtgcagagaggcaagccctgggcagtggcaggcacaggccttcttgaccccagaccttcagcctgtcatattttggctcctccttagtgaaggttgttgagggtgttttgcagagagacatgacgccaatcttaatttttgacaattttccatagcatgcagataatttgtttccaaaacttttcattttcctaaagtcatcttgattggtatcagctatttccataaaacgatcggatgagttttgatggacagatcaggcttttgtttacaactgttttgctcctaatcattccaccacatcacatgtcatggacctgaattgcgtcaagaagacgggcttgtctgtcaggccctggtgggcactttgatagcgggcatgctgtgccatgacacgtgtggtgttgggtcttgctggacaagctgtgctgtgttcagtgtgcggagcctctgctagatgctctcatttggggcactgggccagtgctactgggagcacttctgttttgtgtcactgacatccaatagcatcgttatgtagagcaaacaccgaagggctgcatttctttgtgggcttattctcgagaaaactgggggcagatccctcctcaaggaggggagggccaccttggtttccagtcaagtattgtgaaaattatccaacactcaggcaatccacccaaccctgctgcccatgtctggagaagcaaagtgtcaggggtagtccaggcccacctggagacaggtcaggccctgcagagaaaggtctgacagacgggggtgagggaagaccccccaaaggcctccagagtcccaccaggtctccaggtccttgtcataaccagagaggccccagccccagaggaccaggtcccctgctccactgtccacaggggcccacctgcaagcacactggcagagctcaagaccacacatgcctgcaaggtgaggcctgtctgggctctgtcctcctgcaggccccaggcctgggtggctgggcgaaggcagctgcttatgcagactccagggggaaagccgcctctcatctctggccgtccccaggacgctggatccaccaatatctcaccaacctggagagccactcaacccctcatttcacatgtttgaacatagaggaccagaggggtgtggcctgtctaggaggtcttaggagctcgggtcctgactctgccacttaccagctcttgtctgtcccatgtgcctccgcttcccctcgggacacagagatattgtgaaagttaaacaacataatccccgtaaaacacttcgagcagtgcctggtatctggccagcaagtgatcaatggtgatccattaccatcctgggaccccatcagagccttctgaggtggagggaagggcgtgctggggagcacaggtgcaggtcacaagaaggaagtcagtcccataagccaggtatctaaccccatccctgctcccccaaggtaagggccagcatctaaccccacccctgctcccccaaggtaagggccagcatctaaccccatccctgctcccccaaggtaagggggccagcatctaaccccatccctgctcccccaaggtaagagccagcaacggaggcctgggaggctcctgggttctgggccgcagcgcctctgcgaggtctgcaggcttcgctctaggaggggatgggggctgggcaggtccctgctccagaggaggaggacctgggcctgcggagcgccgcggtgggagtgctggagtcctggcccgtcatccccgtctgccccacagcgaggacgatgctgccactgtataccgcgcagccgcgatgctgaacatgacgggctccgggtacgtgtggctggtcggcgagcgcgagatctcggggaacgccctgcgctacgccccagacggtgagtgctgggccttggcggggtccccgaacggggaggaccccacgggctctgagtcgcatgctcgcctaggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagaggtgggcggggcctccccggagctgggcggggctgctcttggggaggtgggcggggtcactccagagatgggcggggccgctcttggggaggtgggcggggccactctccagagctgggcggagcagctctcaggactaggcggggccgctcttagggagctgggggagcgctcctcaagagatgggtgggggcactctcggggaggtgggcggggtcgcttccaggaggtgggcggcgtcgctctcaggggtactgcagtggagcctgctgccaacatcctctggacactgttacttctctcctctccccccacacccccagcaccaccacatctaatggcacaatcatctgccctcttctcaacactgacaccagtacctgggccgtcactggagtggggactggctccactgcctccgcccctactttccacactgcagcccaccctgaaacagcacccctctccctgtgtggctggcagcctttgggaggaggctcttgatgcagatggggactgaaagcttccagggacccaggaggccagacaagcagcccaagaacagcacacgagccttagacagccagggttggccaaggcccagagacccaagtgaacatctgcagtgtggcaggagttagctcacagcacgcctggacaccatgccatgccagctcacccccagatccccaaccactgagtagcacgtgcagagccaccatccacaacgcccacataagtgcagatgtaggcagcacgtgtgcacacacacgacacacatacacagaaccatgtgtgcacacagactcaggcacatgacacacatgtgacacaagcacatgcatggggtgcaccccacatagggatcacgtgtgcacacaggttcactgatgtggcccgatgggtacaatgcacacgtgcacacacagccggacaggacagcctggtggttagagctggctcaacctcgccttcacttgctggcaagagggcaggcatccttctgagcttctgcctccgtctctgtaaggcaggatggttctgaggacaacgtcctaacccacagaaagccgggtctggcacccataaaccactcagctgtcatgaaccacaccgtccatctggtgcaggcagataccacggtgtccagggtctggcgtctgctgatctttccgttcttgggactgggacaagggaaaagcccaagctgctcagccggcaggagaaggagcagggagaaggagcagggggaaggagcagggagaagcagcagggggaaggagcagggaggagcaggagaaggagcatctctgagaagcctcagctatgcttccttccctagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtaggtgggggtcatgagggggtgggggctggggccttagggtcctggggccaagacccctgcgtggccaccctccatctcatactcccacccccaggtcatccctaatgacaggaagatcatctggccaggcggagagacagagaagcctcgagggtaccagatgtccaccagactgaaggtgggggccccacagacctccctcagtgtccccaccccagacagcccatccaccccctctggcctgaaggaggagggtgcggtgaggtcaatgaaagccactaaaggaagtgggggtggggcctgcttcccctggacaccgtccagcacacctggcacagcacaggaagcagagagaacaggagggaggagaggaagctgcccccatcccacagggggtctccagtgcccctcttgacccagccctacttaagtctggggcagttagttgtctgacaggaccctgctggggaagagcagatgggggacagcaggcagacctcagcttcagcactcgctgtccccagtcctggtcctccacacccctcatccctcctccagcctgcattgctcttgatgggaccgggtcaaactgtcctcttccaccgtgtgggacagcccttcctgactcccctgggcctctgagagcctctgccctcgccggcttcctcctccagaacatctttcccttggctccctactccagggtgctctcctggccattcctccccgggcagagccacactacccccactccacacacactccagtcctggtagcatcacagaccaccaaaggcaaggacctcacaggcgacacgcccaccaaccttctctcggtcattccaagccctcaaatgtctcttgaccctgtctgttttctgagcccacccctgaagcttggtgtcagcccctgtgacctctcacccaggctccctcccctgctctgcaccggcccctgtggcctctcacccaagctcccttccctgctctgcagacagggtggggttttccagtgccagtgtgggtttcattgcagcccagacacctcacactgaaaagtctgaaagcagcggtcaaacgctaatggccaaaaggccccatctaggctgtgagatggagatggctttttacaaatttgtttctggcctcactaattttttaaaaatactagcatatatattacctgtataacaggaaaatgtaatgaaagttttataaagcaaatcaacttcttcaatggctcctgattcccctggggataaaagacaaaatgctgcctggaggctgagggtgggcgggcctgcccccctctcaggctcaccctgcctaggacatgccgggagggtgcctctcccaccacccccacgcctccctgcctttgcagttctggactgcagactcctctgctccacctgccctcaggcacctgcttgatccctgcccaccttgagggctcagctctgacaccatctcccctcacagttctggcagtgtgctatgctctattccagccccctgtgccccagatcccttccccacccccatgccatggtcccttgaaggacagacaggagggcgagcccaagcaggagtgtgggtcgaagaggccacggcgcggtggagcacgtacacacgggcaagagaaaggagccagagacctacattcaaagcctgagggcttcgggactgggggccgggacaggcagtgcgccgggatgaagggaggcacgggtgggtggccccacgggtcccaggtcctgtgcaggtgcagggtcggctttgtggacatgcccctgtcctcgtggcacagcagggtgggggtcagcctgcaggctgggctgtttctcaccccaggaagatgcctggcatacacgggacatcagcggctcctctgctggagggaatcatgtctttttttttttgagacagagtctcgctctgtcgcccaggctggagtgcagtggcgcggtctccgctcactgcaagctccgcctcccgggttcacgccattctcctgcctcagcctcctgagtagctgggactacaggtgcccaccaccacgcccggctaatttttttgtattttcagtagagacggggtttcaccgtgttagccaggatggtctcgatctcctgacctcgtgatccggccgcctcggcctcccaaagtgctgggattacaggcgtgagccacagcacctggcggggaatcatgtctaagccaagactggagaaaaagtggccaagagaagggtccagctctccaggagtcttttctgagcccccagcccccacccccccggggctgcaggcagacgatgctgacggtggctggggaggacgtgtcctgaacacttgggctcgtgaagaagctccagagaggggcagtggccggcggcgcagggcggggggtgtgaggggtgcgtcggggattaagaggggcgccaggggaggctgggagctgagaagagactgccgccctgggcagccttaggtcggtggtccaggctgggtctccccttcccccccagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagccgtgagtgcgcggggcagggcgcggggcgcggggcagggcgcggggcgtggggcggtctggagcccagcagttaccgcccgcacctacccagcccgccacacggtgcctcagtgttgctacggcttttgcatcgacctgctcatcaagctggcacggaccatgaacttcacctacgaggtgcacctggtggcagatggcaagttcggcacacaggagcgggtaggctagacggcgggggtggggaccagcgtgagaggggcctgcaggcgcggtcggagtgggtggggcatggagtaggcggggcttgcagatggtggggggtcctggggtgagtggggcatggagtgagcggagcctgcgggctgggtcctggcgtggataaagcatggggtgggcggggcctgagggctgggtggggcccgacatgggaggggcctgacgtgggggtcggagtgggtggggcacggagtgggcagggcctgcaggcgggggtctggagtgggcggggcctgctggctgtggtggggcccgcccggcgtgggaggggtctgcgagccagggcggggctggagtggggtggggcctgcgagctgggtagggtcttggggagaagacccccggagtgctctagggcggcttcagtcgggggtacctgtggcgggagctgggaggacgctgcctgcatgcccgccggctctgtcgcctcgcaggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggtgggcaggggccgggtggcggggtggcggcggggggagtccctggagggcccgggccgcgctgacctcgcgtccctccgcaggagattccccggagcacgctggactcgttcatgcagccgttccagagcacactgtggctgctggtggggctgtcggtgcacgtggtggccgtgatgctgtacctgctggaccgcttcaggtgagcgcgacccggggctcagacacctccatctgcggggcgcggagccggccaggggcggggcagggccgcctctcccgccctctctcccgcccgccctctgcgccccgcagccccttcggccggttcaaggtgaacagcgaggaggaggaggaggacgcactgaccctgtcctcggccatgtggttctcctggggcgtcctgctcaactccggcatcggggaaggtaaggccccgcccggcccgcctggtcccgcctcggccctctagggtctgacagagccccccgcccgcccacaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcgggtgaggcctggccgggctgggggagggaatgcgaggtgagctggggtcggcctcggttaggggcctggggagccgccgccgcgatccctgccctccgaccctgcagctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaagtgaggcgcgggcggccaccctggcggggcgggacaggtgcggggagggggagggtggcctccaccgggcaggagagcgtccgggccgggcaccccggagggcgcgggcgtggggcttccaggctggcaggaccaaggcccccgtgactccgcctctgccggcagcaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtgagtgtccgtgcgcccgcgtccctcctccgcccctctccgccagaggtggacgccctccccagtgccagaccactccgaggccaccactgatttcccacccaggccgggcgctgcccactccacgccgcaccctaccccgcaggccccgccccggccccgcccccagcttgctccttcccgtcctgggccccgcctcactgcaggctcacttgttcccaccgccaggtcccacgagaatggcttcatggaagacctggacaagacgtgggttcggtatcaggaatgtgactcgcgcagcaacgcccctgcgacccttacttttgagaacatggccggtgcgttctccttcatccattctcgggtgggttctccgtgggctgcggcctccctggccagcaactgaggctctgggtcccggcacacaggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcaggtagggcaggccaccctccgaggcctggtgcccagggcccggcctggccacggccctcctccatccccgaaggccgtggcactggctctggctctggtgggcaggactggagctaggagccatggccaggggcagtggtgagtgctcccagggcacgggggcagcaccggtggggggctgcctgcaggtggctgcccactgcaaagccggggccgagggaggccacgcaccctgctccaagcctccgcctggcccctctgtctccagagtcgcccgccggtacccattccataggaaggcaatcaggcagggtaagacaggggcccgcctgtgtatggcacgtgagtccaagatgcattttgccctccgccgacccaagccccttgacacccttcggagacccccccctttcctgctatgtccttgtgctccgtgactctaatccgaattgggccaggtccggtcctgcctggtgcccaggttgtatccatgagaatttgccaccagcaagggcagccacggcccacctgggacagggtgggcagtgggcctgtacaggcctaagggctcgtggcccgcggtcgagttccggttcactccgtctcttctctttctctgggtgccgtcctggagcctgtgtcctgagatgaagccgacagtgcggccagggctgctgggggatgggggttgctggaggctccacacctctcatccgcccgctcttgctcttggcccccacaggtcccctggggacctggccgctgccagcactggcgggcacaggccacctggccatcagacctgaggccagagtcccgggagctgcctctgtcactccaattccacctcgacacctgcctccagccctcggccccttcctgaatcttggtgtgtgccccttgggggtcagtggcctccacgcagacagctggtgtggcctgaggggcaactcctccagtcctcagaggactcctcctcctcgggacgcctgtaagccagggccacccaggagccagggagccaggcggacctcccaggaagagccagccgagagcccccaagcccagccccagcacgagcaaggtcaggcccgagaccccgggcaggagaagaggccaccctcgaacgtccgctgtcggcccgtctgtccagcacagggaggcaggcaggagcgagggcccaagtggccggccaggctgggcagcggcccatgcaggagcaggcgagggcaggtgtggccaccaccctagccatctaatcacttatacatattcattttaggatagaaagagtggtagagcagagcctgaccctaaaaagaaagccacatttagggctatcacctccaccctggcttccagcttcaagaggcgtaggtcctccaaagacacggtaagggggagagcaccccagtcccgcgtccgactccacctgccctgccctgcgtgtgtctcccgccccatcaccccgccccggaccctgggctcctgtggcccactctgcccctgtctccctgtggcggccgctctgcccagcccgcccatgctgctctctctcactctctggacctttctccccggccctcctgggtcctcggctttccccgtgtgtctccgttagtctgcccgcccacctcccctgccatgacccacacgccatcttgaagcctgtcatctcgttggtcagtcagtcagccacaccacctctcggggccaggtctggggccctgggagcccagcgtggccccatcctggactcctcagctgccgggaggccacaccacttctctgttatgtccccgtttctctcgcctctcccagaggggcccgccgccctcacttcgcccctgcgacggccctggagggggtggctgtgatgtcccatcccgtccgtctgtctggccactggccccgccccccagacacctgtctcacctgtctcaccagagccatgcgtgttgcatcttcatgtggtctctgtgtgggccgggggctgggggccgggcctgggtccgtctgggtggacggctggggcctggagttggaactggccccggccacaggggactgtcaggcagggagtggggtgggaccaaaaggggtggctcccaccccaggctgagcgggggccctgcaggaggtgtggcggcagctcccagagggtctgagaatgggtaggggcggccccacaaaccctggccttcagaacccaggacgacactgaggttcccagacagggaggcctctggaagggaaacgaccacctcagctcctgaccccagcaaccccacaaggcccaccccaaagagccaggccttctgccctttggagcccagaatcccccacctcctgctcggggcagcttgtccctgtagcggatatgcacactcggaccagaggcccccagagcgaacccagccttgctagaggcaccccaggcccaggcaccatggtggggaggggctgcccagagaggcagcggagacctcagccccgtggccaccctgcagtccagggaccagtctggcccacaggaagcccccagcccataagcagcatcaccagagagaagcttacgcccgggggaggaagtgcgatttgcagccacctgcccctcagtgcactggaagcggggcagacctccagggcacagacaggacttggcatcaagcaagccaaatcccgagatgaagccaccagggtgccccaagagggacccatgaggcctggctgctcagcttcctggggaagggacttggcatgcaggatgggtggacagtgagagcctgtaggcctgggggccactggaggctcaaggagcaggtggaagcaccattcctggagccacctctgctgcggaaagcgggcagagctgatgctgcaaagtctgagccaggagtcccgcagggaacagggagggggaatagcgcagggatcgtgggctgggcaggctggggaagagggggtgtccaggcagacaggagaaacagcgatttggggcaggcagccacggggggcaagcacaaatgtcgtgcaggtgatgggccactttcagagggtgacactgggtcccagggccctgcctggagcgaggccaggtgcagctcagagaccctcatggtgccctcccagggacatgttcccagcggaaccctcacccgagcctctctgggcaccagggaccgtcctctggggccagttctggcatcacgtggcatctggggctggccccgccctgcaaggctgaactgtggggggcactgccagctgggggtctgggcaggggagggcagcccagctcccacctggtctctggggctgcgagcttattcagagggaggcgtgggtggggggctcctttgggtagggtggggtcagtccggctgcggagatcccctgcccctgtcctgtggccggtccgggccagggcggcactgggcgctgagggctggggtccctggcggccggcggggccagcgggtattgattgttggttcttatttatagagcaccgggggtggacgcggcgctttgcaaaaccaaaaagacacagtgctgccgcgacgcgctattgagagggaggagggccagctgcagctgtgttcccgtcatagggagagctgagactccccgcccgccctcctctgccccctcccccgcagacagacagacagacggacgggacagcggcccggcccacgcagagccccggagcaccacggggtcgggggaggagcacccccagcctcccccaggctgcgcctgcccgcccgccggttggccggctggccggtccaccccgtcccggccccgcgcgtgcccccagcgtggggctaacgggcgccttgtctgtgtatttctattttgcagcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctc

SEQ ID NO: 13 - DNA, 51 bp, Artificial partial flanking sequence
ggacctggaagtcatctgccaggccygtgatgacagcctccatgcctccca

SEQ ID NO: 14 - DNA, 23 bp, Artificial partial flanking sequence
tccctgctgtgyactgcccaagg

SEQ ID NO: 15 - DNA, 29 bp, Artificial partial flanking sequence
tcctgtaagaaacakcaaggacctcatca

SEQ ID NO: 16 - DNA, 180 bp, Artificial partial flanking sequence
ggcagcctgtgggtccttgtggtgtagggaacggcctgagcccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 17 - DNA, 180 bp, Artificial partial flanking sequence
aggagcgtgtcctatccccggacgcatgcagggcccccaccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 18 - DNA, 180 bp, Artificial partial flanking sequence; identical to SEQ ID NO: 17

SEQ ID NO: 19 - DNA, 180 bp, Artificial partial flanking sequence
aggagcatgtcctatccctggacgcatgcagggcccccaccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 20 - DNA, 180 bp, Artificial partial flanking sequence
aggagcgtgtactaccccagaacgcatgcagggcccccaccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 21 - DNA, 180 bp, Artificial partial flanking sequence
aggagcgtgtactaccccaggacgcatgcagggcccccaccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 22 - DNA, 180 bp, Artificial partial flanking sequence
tggagcgtgtactaccccaggacgcatgcagggcccccaccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 23 - DNA, 180 bp, Artificial partial flanking sequence
aggagcgtgtcctatccccggaccggacgcatgcagggcccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 24 - DNA, 180 bp, Artificial partial flanking sequence
cccacaggagcgtgtactaccccaggacgcatgcagggcccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 25 - DNA, 180 bp, Artificial partial flanking sequence
cccacaggagcgtgtactaccccaggatgcatgcagggcccccacaggagcgtgtactaccccaggacgcatgcagggcccccatgcaggcagcctgcagaccaacactctgcctggccttgagccgtgacctccaggaagggaccccactggaattttatttctctcaggtgcgtgcca

SEQ ID NO: 26 - DNA, 19 bp, Artificial partial flanking sequence
cgccccrgacggtgagtgc

SEQ ID NO: 27 - DNA, 18 bp, Artificial partial flanking sequence
gcgtggggcrgtctggag

SEQ ID NO: 28 - DNA, 16 bp, Artificial partial flanking sequence
gcccggyccgcctggt

SEQ ID NO: 29 - DNA, 20 bp, Artificial partial flanking sequence
gacccccstcctcgggctaa

SEQ ID NO: 30 - DNA, 26 bp, Artificial partial flanking sequence
gaaagatggtgtcrcttttgctattt

SEQ ID NO: 31 - DNA, 20 bp, Artificial partial flanking sequence
cgtcccaccayggtctccac

SEQ ID NO: 32 - DNA, 30 bp, Artificial partial flanking sequence
gctgggcgcctgcctygaccagcactttga

SEQ ID NO: 33 - DNA, 35 bp, Artificial partial flanking sequence
gaagaaaagagccttgggttygactagggaacctg

SEQ ID NO: 34 - DNA, 60 bp, Artificial partial flanking sequence
cctgcaccccccagcatcccccctgcagcccccccagcatctcccctgcacccccagcat

SEQ ID NO: 35 - DNA, 40 bp, Artificial partial flanking sequence
cgctgaagcctgtccacctgaaytggaggcggggcggggc

SEQ ID NO: 36 - DNA, 27 bp, Artificial partial flanking sequence
tgagtagcatatakaattttattgctg

SEQ ID NO: 37 - DNA, 29 bp, Artificial partial flanking sequence
ttgcttgccctctmttgcagaataacaag

SEQ ID NO: 38 - DNA, 25 bp, Artificial partial flanking sequence
catttcccttcygtagaccctctgg

SEQ ID NO: 39 - DNA, 25 bp, Artificial partial flanking sequence
tgatgagaattrtaactgttgttgt

SEQ ID NO: 40 - DNA, 17 bp, Artificial partial flanking sequence
ccctcccyggcgagcgc

(19) United States
(12) Patent Application Publication
(10) Pub. No.: US 2003/0100479 A1
(43) Pub. Date: May 29, 2003

(54) Gene polymorphisms and response to treatment

(76) Inventors: David J. Dow, Stevenage (GB); Benjamin Duncan, Bothell, WA (US); Arlene R. Hughes, Durham, NC (US); Penelope Manasco, Wake forest, NC (US); Sreekumar G. Pillai, Durham, NC (US); Theodre C. Spaulding, Durham, NC (US); Colin F. Spraggs, Stevenage (GB); Michael Stubbins, Harlow (GB); Chun-Fang Xu, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/213,948

(22) Filed: Aug. 7, 2002

Related U.S. Application Data
(60) Provisional application No. 60/313,918, filed on Aug. 21, 2001.
(60) Provisional application No. 60/337,819, filed on Nov. 8, 2001.

(51) Int. Cl. C12Q 1/68; A61K 31/00

(52) U.S. Cl. 514/1; 435/6

(57) ABSTRACT

Correlations between polymorphisms in various genes, and a subject's phenotypic response to treatment with a norepinephrine reuptake inhibitor are described. Methods of screening subjects to aid in the medical treatment of obesity are presented.

### CROSS REFERENCE TO RELATED APPLICATIONS

This application claims priority from U.S. Provisional Application No. 60/313,918 filed Aug. 21, 2001 and U.S. Provisional Application No. 60/337,819 filed Nov. 8, 2001.

### FIELD OF THE INVENTION

The present studies relate to polymorphisms in the norepinephrine transporter (NET1), dopamine receptor 2 (DRD2), dopamine transporter (DAT1), monoamine oxidase B (MAOB), serotonin transporter (5HTT), and NR1-NMDA receptor (NR1) genes, and phenotypes that are associated or correlated therewith. More particularly, the present studies relate to the correlation of polymorphic forms of these genes with the phenotypic response of subjects treated with monoamine reuptake inhibitors.

### BACKGROUND OF THE INVENTION

Being overweight or obese substantially raises an individual's risk of morbidity from hypertension, dyslipidemia, type 2 diabetes, coronary heart disease, and other conditions. Despite the expected medical benefits, many overweight individuals find it difficult to successfully lose weight by diet management alone. Obesity is recognized as a complex multifactorial condition that develops from the interaction of genetic and environmental factors. See, e.g., Clinical Guidelines on the Identification, Evaluation, and Treatment of Overweight and Obesity in Adults, Am. J. Clin. Nutr. 68:899 (1998).

Various pharmaceutical compounds have been utilized in weight loss treatments. Serotonergic agents that inhibit the reuptake of serotonin are reported to act on the hypothalamus to decrease satiety. Fenfluramine and dexfenfluramine, serotonergic agents previously utilized in the United States for the treatment of obesity, have been withdrawn from the U.S. market due to reports of valvular heart disease and primary pulmonary hypertension. (Davidoff et al., Arch Intern Med 161:1429 (2001); Michelakis et al., Am J Med Sci 321:292 (2001); Weissman, Am J Med Sci 321:285 (2001)). Sibutramine (MERIDIA®, Knoll Pharmaceuticals) is a norepinephrine, serotonin and dopamine reuptake inhibitor for use in the management of obesity; side effects reported with sibutramine include hypertension and tachycardia, and dose reduction or discontinuation of treatment is recommended in subjects who experience a sustained increase in blood pressure or pulse rate (2001 PHYSICIANS DESK REFERENCE®, Medical Economics Co., (2000)).

In view of the need for medical weight loss therapies and the potential for adverse events related to such therapies, methods of screening subjects to identify those likely to achieve significant weight loss would be useful in medical management of weight loss. In view of the reported occurrence of alterations in pulse rate and/or blood pressure in subjects treated with monoamine reuptake inhibitors, methods of screening subjects to identify those at higher risk of such side effects would be useful.

### SUMMARY OF THE INVENTION

The present inventors have determined that polymorphisms in the Norepinephrine Transporter (NET1), Dopamine Receptor 2 (DRD2), Dopamine Transporter (DAT1), 5HT transporter (5HTT), NR1 NMDA receptor (NR1), and Monoamine Oxidase B (MAOB) genes are correlated with the response of subjects to treatment with neuronal monoamine reuptake inhibitors, including norepinephrine reuptake inhibitors, dopamine reuptake inhibitors, and serotonin reuptake inhibitors. In particular, the present methods are applicable to medical weight loss treatment using monoamine reuptake inhibitors.

A further aspect of the present invention is a method of screening a human subject, as an aid in predicting response to weight loss treatment with a norepinephrine reuptake inhibitor. The method comprises determining the genotype of the subject at a polymorphic NET1 locus, where one form of the polymorphic locus has been associated with increased weight loss in response to treatment with a norepinephrine reuptake inhibitor (compared to weight loss associated with other polymorphic forms of the locus).

A further aspect of the present invention is a method of screening a human subject as an aid in predicting response to weight loss treatment with a dopamine reuptake inhibitor. The method comprises determining the genotype of the subject at a polymorphic DAT1 locus, where one form of the polymorphic locus has been associated with increased weight loss in response to treatment with a dopamine reuptake inhibitor (compared to weight loss associated with other polymorphic forms of the locus).

A further aspect of the present invention is a method of screening a human subject as an aid in predicting response to weight loss treatment with a norepinephrine reuptake inhibitor. The method comprises determining the genotype of the subject at a polymorphic NR1 locus, where one form of the polymorphic locus has been associated with increased weight loss in response to treatment with a norepinephrine reuptake inhibitor (compared to weight loss associated with other polymorphic forms of the locus).

A further aspect of the present invention is a method of screening a human subject as an aid in predicting response to weight loss treatment with a neuronal monoamine reuptake inhibitor. The method comprises determining the genotype of the subject at a polymorphic 5HTT locus, where one form of said polymorphic locus has been associated with increased weight loss in response to treatment with a neuronal monoamine reuptake inhibitor (compared to weight loss associated with other polymorphic forms of the locus).

A further aspect of the present invention is a method of treating a human subject with a neuronal monoamine reuptake inhibitor for weight loss. The method comprises determining the genotype of the subject at a polymorphic locus in the NET1, DAT1, NR1 and 5HTT genes, where one form of the polymorphic locus has been associated with increased weight loss in response to treatment with a neuronal monoamine reuptake inhibitor (compared to weight loss associated with another polymorphic form of that locus), and administering the neuronal monoamine reuptake inhibitor to the subject if the genotype associated with increased weight loss is detected.

A further aspect of the present invention is a method of identifying human genotypes associated with increased weight loss in response to treatment with a neuronal monoamine reuptake inhibitor for weight loss. The method comprises, in a plurality of test subjects, determining the genotype of each subject at a polymorphic locus in the NET1, DAT1, NR1, or 5HTT gene. An effective weight loss regime of a neuronal monoamine reuptake inhibitor is administered to each test subject, and the weight change of each subject is measured. The genotypes of the test subjects are correlated with the extent of weight loss, to identify genotypes associated with increased weight loss (compared to average weight loss in the population).

A further aspect of the present invention is a method of screening a human subject as an aid in predicting response to treatment with a neuronal monoamine reuptake inhibitor. The method comprises determining the genotype of the subject at a polymorphic MAOB locus, where one form of the polymorphic locus has been associated with increased diastolic blood pressure changes in response to a therapeutic regimen of the reuptake inhibitor (compared to changes associated with other polymorphic forms of the locus).

A further aspect of the present invention is a method of screening a human subject as an aid in predicting response to treatment with a neuronal monoamine reuptake inhibitor. The method comprises determining the genotype of the subject at a polymorphic DRD2 locus, where one form of the polymorphic locus has been associated with increased heart rate changes in response to a neuronal monoamine reuptake inhibitor (compared to heart rate changes associated with other polymorphic forms of the locus).

A further aspect of the present invention is a method of screening a subject in need of weight loss treatment, as an aid in predicting weight loss in response to treatment with a neuronal monoamine reuptake inhibitor. The method comprises determining the subject's genotype at the NET1 T342C, NET1 G155A, or NR1 G6435A polymorphic locus. Detection of a genotype selected from NET1 T342C (C/C), NET1 G155A (A/A) and NR1 G6435A (A/A) indicates the subject is likely to achieve greater weight loss in response to treatment (compared to weight loss expected in subjects with alternate genotypes).

A further aspect of the present invention is a method of screening a subject in need of treatment with a neuronal monoamine reuptake inhibitor, as an aid in predicting heart rate increase in response to treatment. The method comprises determining the subject's genotype at the DRD2 C12121T polymorphic locus, where detection of the DRD2 C12121T (T/T) allele indicates the subject is likely to experience a greater heart rate increase (compared to heart rate increase expected in subjects with alternate genotypes).

A further aspect of the present invention is a method of screening a subject in need of treatment with a neuronal monoamine reuptake inhibitor, as an aid in predicting heart rate increase in response to treatment with said reuptake inhibitor. The method comprises determining the subject's genotype at the DRD2 C12121T polymorphic locus and the MAOB G644A polymorphic locus, where detection of the DRD2 C12121T (T/T) allele and the MAOB G644A (G,G) allele indicates the subject is likely to experience a greater heart rate increase (compared to heart rate increase expected in subjects with alternate genotypes).

A further aspect of the present invention is a method of treating a plurality of subjects in need of weight loss treatment. The method comprises determining, in each subject, the genotype at the NET1 T342C, NET1 G155A, or NR1 G6435A polymorphic locus, and administering a norepinephrine reuptake inhibitor to subjects in which the NET1 T342C (C/C), NET1 G155A (A/A) or NR1 G6435A (A/A) genotype is detected.

A further aspect of the present invention is a method of treating a plurality of subjects in need of weight loss treatment. The method comprises determining, in each subject, the genotype at the DRD1 C12121T polymorphic locus and administering a neuronal monoamine reuptake inhibitor to subjects having the DRD2 C12121T (C,T) or (C,C) genotype.

A further aspect of the present invention is a method of treating a plurality of subjects in need of weight loss treatment. The method comprises determining the genotype of each subject at the DRD1 C12121T polymorphic locus, and determining the genotype in each subject at least one of the NET1 T342C, NET1 G155A, and NR1 G6435A polymorphic loci. A therapeutic weight-loss regime of a neuronal monoamine reuptake inhibitor is then administered to subjects having the NET1 T342C (C/C), NET1 G155A (A/A) or NR1 G6435A (A/A) genotype, but not having the DRD1 C1221T (C/C) genotype.

A further aspect of the present invention is a method of administering a neuronal monoamine reuptake inhibitor for medical treatment, to increase the average efficacy of the medical treatment. The method comprises selecting, based on genotype status, a treatment population from a larger starting population of subjects in need of such treatment. The treatment population is selected to increase the percentage of subjects in the treatment population who have a genotype that has been associated with increased efficacy in response to medical treatment with a neuronal monoamine reuptake inhibitor for a defined medical condition. Alternatively, the treatment population is selected to decrease the percentage of subjects in the treatment population who have a genotype that has been associated with increased risk of adverse side effects. The reuptake inhibitor is then administered to the selected treatment population, thereby enhancing the average response to the medical treatment (or decreasing the average incidence or severity of a side effect) compared to that which would have been expected to occur had the compound been administered to the larger starting population. The ‘selection’ may occur by any suitable process as would be apparent to those skilled in the art. Examples of suitable selection methods include genetically screening starting population subjects, or otherwise classifying subjects by genotype (e.g., where a subject's genotype is known, genetic testing need not be repeated); or otherwise regulating access to the pharmaceutical neuronal monoamine reuptake inhibitor, to increase the number of subjects in the treatment population who have genotypes that have been associated with increased average weight loss, or a decreased incidence of an adverse side effect. Exemplary genotypes associated with increased average weight loss in response to a norepinephrein/dopamine reuptake inhibitor (e.g., GW320659) include NET1 G155A (A,A); NET1 T342C (C/C); NET1C120A (A/A); DAT1 VNTR (9,9); DAT VNTR (10,9); NR1 G1001C (G/C); NR1 G6435A (A/A); 5HTT G769 (G/G); and 5HTT G160A (A/A); exemplary genotypes associated with a decreased incidence of cardiovascular side effects include DRD2 C12121T (C/C), DRD2 C12121T (T/C) and MAOB G644A (A/A).

A further aspect of the present invention is a method of administering a neuronal monoamine reuptake inhibitor to decrease the incidence of adverse side effects. The method comprises selecting, based on genotype status, a treatment population from a larger starting population of subjects in need of such treatment. The treatment population is selected to decrease the percentage of subjects in the treatment population who have a genotype that has been associated with increased risk of adverse side effects. The reuptake inhibitor is then administered to the selected treatment population, thereby decreasing the incidence of the side effect compared to the incidence that would have been expected to occur had the compound been administered to the larger starting population. The ‘selection’ may occur by any suitable process as would be apparent to those skilled in the art. Examples of suitable selection methods include genetically screening starting population subjects, or otherwise classifying subjects by genotype (e.g., where a subject's genotype is known, genetic testing need not be repeated); or otherwise regulating access to the pharmaceutical compound to increase the number of subjects in the treatment population who have genotypes that have been associated with a reduced risk of an adverse side effect. Exemplary genotypes associated with a decreased incidence of cardiovascular side effects in response to a norepinephrein/dopamine reuptake inhibitor (e.g., GW320659) include include DRD2 C12121T (C/C), DRD2 C12121T (T/C) and MAOB G644A (A/A).

A further aspect of the present invention is a method of treating subjects in need of medical weight loss treatment, by administering GW320659 to subjects having a genotype selected from DRD2 C12121T (C/C), DRD2 C12121T (T/C) and MAOB G644A (A/A).

A further aspect of the present invention is a method of treating subjects in need of medical weight loss treatment, by administering GW320659 to subjects having a genotype selected from NET1 G155A (A,A); NET1 T342C (C/C); NET1C120A (A/A); DAT1 VNTR (9,9); DAT VNTR (10,9); NR1 G1001C (G/C); NR1 G6435A (A/A); 5HTT G769 (G/G); and 5HTT G160A (A/A).

### BRIEF DESCRIPTION OF THE FIGURES

FIG. 1 graphs the mean absolute change in body weight (in Kg) at 24 weeks of treatment for subjects in the 15 mg/day and placebo dosage groups, according to genotype at the NET1T342C loci (1, 1 or 1, 2 or 2,2). Largest weight change was seen for subjects in the 15 mg/day dosage group who had the 2,2 genotype.

FIG. 2 graphs the mean absolute change in body weight (in Kg) at 24 weeks of treatment for subjects in the 15 mg/day and placebo dosage groups, according to genotype at the NET1G155A loci (1, 1 or 1, 2 or 2,2). Largest weight change was seen for subjects in the 15 mg/day dosage group who had the 2,2 genotype.

FIG. 3 graphs the mean absolute change in body weight (in Kg) at 24 weeks of treatment for subjects in the 15 mg/day and placebo dosage groups, according to genotype at the NR1G6435A loci (1, 1 or 1, 2 or 2,2). Largest weight change was seen for subjects in the 15 mg/day dosage group who had the 2,2 genotype.

FIG. 4 is a chart showing the significance of weight loss differences between placebo treatment and treatment with GW320659 (15 mg/day), after 24 weeks of treatment, for different genetic polymorphisms. (PBO=placebo, GW=treatment with GW320659)

FIG. 5 graphs the mean absolute change in body weight (in Kg) at 24 weeks of treatment for subjects in the 15 mg/day and placebo dosage groups, according to genotype at the NET1C120A loci (1, 1 or 1, 2 or 2,2). Largest weight change was seen for subjects in the 15 mg/day dosage group who had the 2,2 genotype.

FIG. 6 graphs the mean absolute change in body weight (in Kg) at 24 weeks of treatment for subjects in the 15 mg/day and placebo dosage groups, according to genotype at the NR1G1001C loci (1, 1 or 1, 2 or 2,2). Largest weight change was seen for subjects in the 15 mg/day dosage group who had the 1,2 genotype; however, no subjects had the 2,2 genotype.

FIG. 7 graphs the mean absolute change in body weight (in Kg) at 24 weeks of treatment for subjects in the 15 mg/day and placebo dosage groups, according to genotype at the 5HTTG769T loci (1, 1 or 1, 2 or 2,2). Largest weight change was seen for subjects in the 15 mg/day dosage group who had the 1,1 genotype.

FIG. 8 graphs the mean absolute change in body weight (in Kg) at 24 weeks of treatment for subjects in the 15 mg/day and placebo dosage groups, according to genotype at the 5HTTDel-Ins loci (1, 1 or 1, 2 or 2,2). Largest weight change was seen for subjects in the 15 mg/day dosage group who had the 1,2 genotype.

FIG. 9 graphs the mean absolute change in body weight (in Kg) at 24 weeks of treatment for subjects in the 15 mg/day and placebo dosage groups, according to genotype at the 5HTTG160A loci (1, 1 or 1, 2 or 2,2). Largest weight change was seen for subjects in the 15 mg/day dosage group who had the 2,2 genotype.

FIG. 10 graphs the mean absolute change in body weight (in Kg) at 24 weeks of treatment for subjects in the 15 mg/day and placebo dosage groups, according to genotype at the DAT1VNTR loci (10,10 or 10, 9 or 9,9). Largest weight change was seen for subjects in the 15 mg/day dosage group who had the 9,9 genotype.

FIG. 11 graphs the change in Food Craving Inventory score for a subgroup of subjects having the NET1C120A 1, 1 or 2,2 genotype and who weighed >86.6 kg at baseline. Subjects are compared among the placebo dosage group, the combined 2.5 mg/day+5.0 mg/day dosage group, and the combined 10.0 mg/day+15 mg/day dosage group. (Subgroup represented by gray bars; non-subgroup by hatched bars). Largest change was seen in the subgroup, at highest dosage. Results are expressed as mean, with 95% confidence intervals.

FIG. 12 graphs the overall mean time adjusted change in Heart Rate (in beats per minute) for the subgroup of subjects having the DRD2 C12121T 2,2 genotype, compared among the placebo dosage group, the combined 2.5 mg/day+5.0 mg/day dosage group, and the combined 10.0 mg/day+15 mg/day dosage group. (Subgroup represented by gray bars; non-subgroup by hatched bars). Largest change was seen in the subgroup, at highest dosage. Results are expressed as mean, with 95% confidence intervals.

FIG. 13 graphs the overall mean time adjusted change in Diastolic Blood Pressure (in mmHg) for the subgroup of subjects who are not 1,2 at the DRD2C12121T locus (i.e., who are either 1, 1 or 2,2); compared between the placebo+2.5 mg/day+5.0 mg/day combined dosage group and the 15 mg/day treatment group. (Subgroup represented by gray bars; non-subgroup by hatched bars). Largest change was seen in the subgroup, at highest dosage. Results are expressed as mean, with 95% confidence intervals.

FIG. 14 graphs the change in weight (in Kg) at 24 weeks for a genetically defined subgroup (individuals who were 2,2 for NET1T342C and/or 2,2 for NET1G155A and/or 2,2 for NR1G6435A). In the combined 10 mg/day+15 mg/day dosage group, mean weight loss was 6.05 kg, and significantly greater than placebo. (Subgroup represented by gray bars; non-subgroup by hatched bars). Largest change was seen in the subgroup, at highest dosage. Results are expressed as mean, with 95% confidence intervals.

FIG. 15 graphs the overall mean time-adjusted change in Diastolic Blood Pressure (DBP, in mmHg) for a genetically defined subgroup (individuals who were 2,2 for NET1T342C; and/or 2,2 for NET1G155A; and/or 2,2 for NR1G6435A), showing an increase in DBP (mean rise 2.4 mm Hg) in the combined 10 mg/day+15 mg/day dosage group that was not significantly different than that seen with placebo. (Subgroup represented by gray bars; non-subgroup by black bars). Results are expressed as mean with 95% confidence intervals.

FIG. 16 graphs the overall mean time-adjusted change in Heart Rate (HR; in beats per minute) for a genetically defined subgroup (2,2 for NET1T342C; and/or 2,2 for NET1G155A; and/or 2,2 for NR1G6435A), showing an increase in HR (mean rise 4.7 beats per minute) in the combined 10 mg/day+15 mg/day dosage group that was not significantly different than that seen with placebo. (Subgroup represented by gray bars; non-subgroup by black bars). Results are expressed as mean with 95% confidence intervals.

FIG. 17 graphs the change in weight (in Kg) at 24 weeks for a genetically defined subgroup (DAT1VNTR=10, 9 or 9,9), showing a mean weight loss for the subgroup, at the 15 mg/day dosage, of 6.89 kg. (Subgroup represented by gray bars; non-subgroup by black bars). Results are expressed as mean with 95% confidence intervals.

FIG. 18 graphs the overall mean time-adjusted change in Heart Rate (in beats per minute) for a genetically defined subgroup (DRD2C12121T=2,2 and MAOBG644A=1,1), showing a mean increase in HR of 13 beats per minute in the combined 10 mg/day+15 mg/day group. (Subgroup represented by gray bars; non-subgroup by hatched bars). Results are expressed as mean with 95% confidence intervals.

FIG. 19 graphs the change in weight (in Kg) for a genetically defined subgroup (individuals who are 2,2 for NET1T342C and who are not 2,2 for DRD2C12121T), divided by dosage groups (placebo, 2.5 mg/day+5.0 mg/day, and 10 mg/day+15 mg/day). Largest change was seen for subjects in the subgroup, at the higher dosage. (Subgroup represented by gray bars; non-subgroup by hatched bars). Results are expressed as mean with 95% confidence intervals.

FIG. 20 graphs the change in Food Craving Inventory for a genetically defined subgroup (individuals who are 2,2 for NET1T342C and who are not 2,2 for DRD2C12121T), divided by dosage groups (placebo, 2.5 mg/day+5.0 mg/day, and 10 mg/day+15 mg/day). Largest change was seen for subjects in the subgroup, at the higher dosage. (Subgroup represented by gray bars; non-subgroup by hatched bars). Results are expressed as mean with 95% confidence intervals.

FIG. 21 graphs the overall mean time-adjusted change in Heart Rate (in beats per minute) for a genetically defined subgroup (individuals who are 2,2 for NET1T342C and who are not 2,2 for DRD2C12121T), divided by dosage groups (placebo, 2.5 mg/day+5.0 mg/day, and 10 mg/day+15 mg/day). (Subgroup represented by gray bars; non-subgroup by hatched bars). Results are expressed as mean with 95% confidence intervals.

FIG. 22 graphs the overall mean time adjusted change in Systolic Blood Pressure (in mmHg) for a genetically defined subgroup (individuals who are 2,2 for NET1T342C and who are not 2,2 for DRD2C12121T), divided by dosage groups (placebo, 2.5 mg/day+5.0 mg/day, and 10 mg/day+15 mg/day). (Subgroup represented by gray bars; non-subgroup by hatched bars). Results are expressed as mean with 95% confidence intervals.

FIG. 23 graphs the overall mean time-adjusted change in Diastolic Blood Pressure (in mmHg) for a genetically defined subgroup (individuals who are 2,2 for NET1T342C and who are not 2,2 for DRD2C12121T), divided by dosage groups (placebo, 2.5 mg/day+5.0 mg/day, and 10 mg/day+15 mg/day). (Subgroup represented by gray bars; non-subgroup by hatched bars). Results are expressed as mean with 95% confidence intervals.

FIG. 24 graphs the overall mean time-adjusted change in Diastolic Blood Pressure (in mmHg) for women according to genotype at the MAOBG644A polymorphic site (1,1; 1, 2 or 2,2), and according to dosage group (combined 10 mg/day+15 mg/day or 15 mg/day). Smallest changes were seen in the 2,2 genotype.

FIG. 25 graphs the mean weight change (in Kg) at 24 weeks for subjects in the 10 mg/day+15 mg/day combined dosage group, according to genotype at the NET1T342C loci (1, 1 or 1, 2 or 2,2), and ethnicity (all ethnic groups, Caucasians). Largest weight change was seen for subjects having the 2,2 NET1T342C genotype.

FIG. 26 graphs the mean weight change (in Kg) at 24 weeks for subjects in the 10 mg/day+15 mg/day combined dosage group, according to genotype at the NET1C120A loci (1, 1 or 1, 2 or 2,2), and ethnicity (all ethnic groups, Caucasians). Largest weight change was seen for subjects having the 2,2 NET1C120A genotype.

FIG. 27 graphs the change in supine heart rate (in beats per minute) for subjects in the 15 mg/day dosage group, and in the combined (10 mg/day+15 mg/day) dosage group, according to genotype at the DRD1C12121T loci (1, 1 or 1,2 or 22). Change is measured in beats per minute; largest change was seen in the 2,2 genotype.

FIG. 28 graphs the change in supine diastolic blood pressure (in mmHg) for subjects in the 15 mg/day dosage group, and in the combined (10 mg/day+15 mg/day) dosage group, according to genotype at the DRD1C12121T loci (1, 1 or 1,2 or 22).

FIG. 29 graphs the change in supine heart rate (in beats per minute) for subjects in the 15 mg/day dosage group, and in the combined (10 mg/day+15 mg/day) dosage group, according to genotype at the 5HTT T3287C loci (1, 1 or 1,2 genotype).

### DETAILED DESCRIPTION OF THE INVENTION

The present invention is concerned with the pharmaceutical treatment of weight and obesity, particularly the use of neuronal reuptake inhibitors of norepinephrine, serotonin and/or dopamine, and more particularly with the use of GW320659, for weight loss in subjects in need of such treatment. The present inventors have determined that polymorphic variations in the NET1, DRD2, DAT1, MAOB, 5HTT, and NR1 genes can be correlated to, or associated with, phenotypic responses to such pharmaceutical treatment.

In the present studies, genetic samples were obtained from subjects enrolled in a clinical trial of GW320659 for weight loss. The genetic samples were screened for the presence of various polymorphisms (as defined herein), using technologies as are known in the art.

The present invention is further concerned with alterations in blood pressure and pulse rate that have been associated with the pharmaceutical use of monoamine neuronal reuptake inhibitors, including but not limited to the use of such compounds for the treatment of obesity. Such compounds include neuronal reuptake inhibitors of norepinephrine, serotonin and/or dopamine, such as GW320659. The present inventors have determined that polymorphic variations in the NET1, DRD2, 5HTT, NR1, and MAOB genes can be correlated to, or associated with, phenotypic responses to such pharmaceutical treatment.

NET1:

The norepinephrine transporter protein (NET) is the presynaptic reuptake site for norepinephrine and is a site of action for several drugs with CNS effects. NET1 is a member of a family of Na/Cl dependent neurotransmitter proteins which share sequence similarity, including NET1, DAT1 and 5HTT. The transmembrane domains of NET1, DAT1 and 5HTT show a high degree of sequence similarity in transmembrane domains 1, 2 and 4-8. The NET transporter is encoded by 14 exons spanning 45 kb. A further exon identified in the 3′ region gives rise to shorter splice variants and an altered C terminus associated with a lack of transport. (Biochim Biophys Acta 1398:365 (1998)).

NET1 is also known as the Solute Carrier Family 6 (neurotransmitter transporter, noradrenalin), member 2 (SLC6A2).

Polymorphisms in the NET1 gene have been identified by Stober et al., who reported 13 DNA sequence variants including five missense substitutions. The missense substitutions Val69Ile, Thr99Ile, Val245Ile, Val449Ile, and Gly478Ser are located at putative transmembrane domains (TMD) 1, 2, 4, 9, and 10, respectively. A highly polymorphic silent 1287G/A polymorphism was also reported. Stober et al., Am. J. Med. Genet 67:523 (1996); Stober et al., Am. J. Med. Genet. 88:158 (1999). See also Bonisch et al., J. Autonomic Pharmacol. 19:327 (1999).

The NET1 polymorphisms assayed in the present study are shown in Table 1. An amino acid and complete coding region sequence (mRNA) for NET1 is provided at Genbank Accession No. NM 001043. The NET1 G155A polymorphic site is shown in the sequence (exons 9-10) provided at Genbank Accession No. X91127 (SEQ ID NO:1; nucleotide position 155 therein corresponds to the NET1 G155A polymorphic site). The NET1 T342 polymorphic site is shown in the sequence (exon 13-15) provided at Genbank Accession No. X91119 (SEQ ID NO:2; nucleotide position 342 therein corresponds to the NET1 T342C polymorphic site). The NET1 C120A polymorphic site is shown in the sequence (exon 8) provided at Genbank Accession No. X91126 (SEQ ID NO:3; nucleotide position 120 therein corresponds to the NET1 C120A polymorphic site).

DAT1:

The dopamine transporter protein (DAT1, also known as SLC6A3) is involved in the presynaptic uptake of dopamine by the dopaminergic neurons. The DAT1 gene contains a 40 base pair Variable Number Tandem Repeat (VNTR) polymorphism in the 3′ untranslated region of the gene; up to 11 copy alleles of DAT1 have been described. (See, e.g., Sano et al., Hum. Genet. 91:405 (1993); Vandenbergh et al. Genomics 14:1104 (1992); Byerley et al., Hum. Mol. Genet. 2:335 (1993); Winsberg et al., J. Amer. Acad. Child. Adolesc. Psychiatry 38:1474 (1999); Heinz et al., Neuropsychopharmacology 22:133 (2000)). Between three and eleven copies of the 40-basepair repeat element have been reported in various populations. See e.g., Inada et al., Am. J. Med. Genet. 67:406 (1996). Methods of detecting the number of repeats of this VNTR are known in the art (see e.g., Sano et al., Hum. Genet. 91:405 (1993); Mercier et al., J. Neurol. 246:45 (1999)).

An amino acid and complete coding region sequence (mRNA) for DAT1 is provided at Genbank Accession No. M95167 (SEQ ID NO:4). The DAT1 VNTR region is represented in SEQ ID NO:4 at nucleotides 2741-3140, showing ten repeats of the 40-base pair segment.

MAOB:

The monoamine oxidase B (MOAB) is a catabolic enzyme of dopamine. A G/A polymorphism has been identified in exon 13 of the MOAB gene (G644A). An amino acid and mRNA sequence for human MAOB is provided at Genbank Accession No. XM 010261. A sequence for exon 13 is provided at Genbank Accession No. Z29071 (SEQ ID NO:5; nucleotide position 644 therein corresponds to the MAOB G644A polymorphic site).

DRD2:

The dopamine receptor D2 (DRD2) is involved in dopaminergic transmission. Various polymorphisms of the DRD2 gene have been reported in the literature. See, e.g., Jones and Peroutka, Neuropharmacology 37:803 (1998); J. Biol. Chem. 271:26013 (1996).

The present inventors screened for the polymorphisms shown in Table 1. An amino acid and complete coding sequence for human DRD2 is provided at Genbank Accession No. AF050737 (SEQ ID NO:6; nucleotide position 12121 therein corresponds to the DRD2 C12121T polymorphic site; nucleotide position 20236 therein corresponds to the DRD2 C20236T polymorphic site; nucleotide position 32806 therein corresponds to DRD2 C32806T polymorphic site).

5HTT:

The human 5HTT is encoded by a single gene (SLC6A4) found on chromosome 17q12 (Ramamoorthy et al., Proc. Natl. Acad. Sci. USA 90:2542 (1993); Gelernter et al., Hum. Genet. 95:677 (1995). The 5HT transporter regulates the magnitude and duration of serotonergic responses. An insertion/deletion polymorphism consisting of a 44 base pair segment in the transcriptional control region 5′ upstream to the 5HTT coding sequence has previously been identified. The deletion (or short) allele of this polymorphism is associated with decreased transcription efficiency of the 5HTT gene promoter, decreased gene expression, and decreased 5-hydroxytryptamine uptake. (Heils et al., J. Neural Transm. 102:247 (1995); Heils et al., J. Neurochem 66:2621 (1996), Lesch et al., Science 274:1527 (1996)). Variation in functional 5HTT expression due to 5HTT promoter polymorphism has been implicated as a potential genetic susceptibility factor for affective disorders (see, e.g., Furlong et al., Am J Med Genet Feb. 7, 1998;81(1):58-63; Menza et al., J Geriatr Psychiatry Neurol 1999 Summer;12(2):49-52; and Rosenthal et al., Mol Psychiatry 1998 Mar;3(2):175-7.) The 5HTT polymorphisms assayed in the present study are shown in Table 1.

A nucleotide sequence for exon 1 of human 5HTT is provided at Genbank Accession No. X76753 (SEQ ID NO:7; nucleotide position 623 therein corresponds to the 5HTT T623C polymorphic site; the 44-base pair 5HTT insert/deletion polymorphic site is represented at nucleotide positions 1826-1869; and nucleotide position 3287 corresponds to the 5HTT T3287C polymorphic site).

A nucleotide sequence for exons 1B and 2 of human 5HTT is provided at Genbank Accession No. U79746 (SEQ ID NO:8; nucleotide position number 867 therein corresponds to the 5HTT C867T polymorphic site; nucleotide position number 2631 therein corresponds to 5HTT A2631 C polymorphic site).

A nucleotide sequence for exons 9 and 10 of human 5HTT is provided at Genbank Accession No. X76758 (SEQ ID NO:9; nucleotide position number 160 therein corresponds to the 5HTT G160A polymorphic site).

A nucleotide sequence for exon 14 of human 5HTT is provided at Genbank Accession No. X76762 (SEQ ID NO:10; nucleotide position number 769 therein corresponds to the 5HTT G769T polymorphic site).

NR1 (NMDA Receptor-GRIN 1)

The N-methyl-D-aspartate (NMDA) receptor (NR1) gene encodes RNA that is alternatively spliced to generate at least seven variants that arise from alternative splicing of three exons: one encodes a 21-amino acid insert in the N-terminal domain; two encode adjacent sequences of 37 and 38 amino acids in the C-terminal domain. Polymorphisms which affect splicing may affect the function of the expressed receptor. (Okabe et al., J.Neuroscience 19:7781 (1999); Hisatsune et al., J. Biol. Chem. 272:20805; Rice et al., Mol. Psychiatry 6:274 (2001); Am. J. Hum. Genet. 65(4) Suppl Poster 1474.

A nucleotide sequence for exons 1 and 2 of human NR1(NMDA) is provided at Genbank Accession No. Z32772 (SEQ ID NO:11; nucleotide position number 1001 therein corresponds to the NR1 G1001C polymorphic site).

A nucleotide sequence for exons 6-21 of human NR1 (NMDA) is provided at Genbank Accession No. Z32774 (SEQ ID NO:12; nucleotide position number 1970 therein corresponds to the NR1A1970G polymorphic site; nucleotide position number 6435 therein corresponds to the NR1 G6435A polymorphic site; nucleotide position number 7701 therein corresponds to the NR1 C7701T polymorphic site.

As is well known genetics, nucleotide and amino acid sequences obtained from different sources for the same gene may vary both in the numbering scheme and in the precise sequence. Such differences may be due to inherent sequence variability within the gene and/or to sequencing errors. Accordingly, reference herein to a particular polymorphic site by number (e.g., NET1 T342C) will be understood by those of skill in the art to include those polymorphic sites that correspond in sequence and location within the gene, even where different numbering/nomenclature schemes are used to describe them.

Flanking Sequences

Table 1 provides a short sequence surrounding each of the polymorphisms screened for in the present studies.

TABLE 1

| Gene | Allelic | Allele | Allele | |
|---|---|---|---|---|
| Polymorphism | Location | 1 | 2 | Sequence Flanking Polymorphism |

NET1 (SLC6A)

| | | | | | |
|---|---|---|---|---|---|
| G155A | Exon 9 | G | A | GGACCTGGAAGTCATCTGCCAGGCCYGTGATGACAGCCTCCAT | (SEQ ID NO:13) |
| | | | | GCCTCCCA (Comp) | |
| T342C | Intron 13 | T | C | TCCCTGCTGTGYACTGCCCAAGG | (SEQ ID NO:14) |
| C120A | Intron 7 | C | A | TCCTGTAAGAAACAKCAAGGACCTCATCA (Comp) | (SEQ ID NO:15) |

DAT1 (SLC6A2)

| | | | | |
|---|---|---|---|---|
| VNTR | * | * | ggcagcctgt gggtccttgt ggtgtaggga acggcctgag | (SEQ ID NO:16) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (1) | |
| | | | aggagcgtgt cctatccccg gacgcatgca gggcccccac | (SEQ ID NO:17) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (2) | |
| | | | ggagcgtgt cctatccccg gacgcatgca gggcccccac | (SEQ ID NO:18) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (3) | |
| | | | aggagcatgt cctatccctg gacgcatgca gggcccccac | (SEQ ID NO:19) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (4) | |
| | | | aggagcgtgt actaccccag aacgcatgca gggcccccac | (SEQ ID NO:20) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (5) | |
| | | | aggagcgtgt actaccccag gacgcatgca gggcccccac | (SEQ ID NO:21) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (6) | |
| | | | tggagcgtgt actaccccag gacgcatgca gggcccccac | (SEQ ID NO:22) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (7) | |
| | | | aggagcgtgt cctatccccg gaccggacgc atgcagggcc | (SEQ ID NO:23) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (8) | |
| | | | cccacaggag cgtgtactac cccaggacgc atgcagggcc | (SEQ ID NO:24) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (9) | |
| | | | cccacaggag cgtgtactac cccaggatgc atgcagggcc | (SEQ ID NO:25) |
| | | | cccacaggag cgtgtactac cccaggacgc atgcagggcc | |
| | | | cccatgcagg cagcctgcag accaacactc tgcctggcct | |
| | | | tgagccgtga cctccaggaa gggaccccac tggaatttta | |
| | | | tttctctcag gtgcgtgcca; (10) | |

NR1 (GRIN)

| | | | | | |
|---|---|---|---|---|---|
| A1970G | | A | G | CGCCCCRGACGGTGAGTGC; | (SEQ ID NO:26) |
| G6435A | | G | A | GCGTGGGGCRGTCTGGAG; | (SEQ ID NO:27) |
| C7701T | | C | T | GCCCGGYCCGCCTGGT; | (SEQ ID NO:28) |
| G1001C | | G | C | GACCCCCSTCTCGGGCTAA (Comp); | (SEQ ID NO:29) |
| MAOB | | | | | |
| G644A | | G | A | GAAAGATGGT GTCRCTTTTG CTATTT; | (SEQ ID NO:30) |
| DRD2 | | | | | |
| C20236T | Exon 6 | C | T | CGTCCCACCAYGGTCTCCAC; | (SEQ ID NO:31) |
| (NcoI) | | | | | |
| C32806T | Intron 8 | C | T | GCTGGGCGCCTGCCTYGACCAGCACTTTGA; | (SEQ ID NO:32) |
| (TaqA) | | | | | |
| C12121T | Intron 2 | C | T | GAAGAAAAGAGCCTTGGGTTYGACTAGGGAACCTG; | (SEQ ID NO:33) |
| (TaqD) | | | | | |
| 5HTT | | | | | |
| Del/Ins | | 528 | 484 | CCTGCACCCCCCAGCATCCCCCCTGCAGCCCCCCCAGCATCTC | (SEQ ID NO:34) |
| | | (Ins) | (Del) | CCCTGCACCCCCAGCAT | |
| T623C | | T | C | CGCTGAAGCC TGTCCACCTG AAYTGGAGGCGGGGCGGGGC | (SEQ ID NO:35) |
| G769T | | G | T | TGAGTAGCATATAKAATTTTATTGCTG; | (SEQ ID NO:36) |
| A2631C | | A | C | TTGCTTGCCCTCTMTTGCAGAATAACAAG; | (SEQ ID NO:37) |
| C867T | | C | T | CATTTCCCTTCYGTAGACCCTCTGG; | (SEQ ID NO:38) |
| G160A | | G | A | TGATGAGAATTRTAACTGTTGTTGT; | (SEQ ID NO:39) |
| T3287C | | T | C | CCCTCCCUGGCGAGCGC; | (SEQ ID NO:40) |

*DAT1 VNTR polymorphism comprised from one to ten repeats; possible genotypes were combinations of alleles 1 (one repeat) through 10 (ten repeats). Flanking sequences are provided for each of alleles 1-10.
“Comp” indicates complementary sequence.

GW320659 Compound

The phenylmorpholinol compound (2S,3S,5R)-2-(3,5-difluorophenyl)-3,5-dimethyl-2-morpholinol (GW320659) is most typically prepared and isolated as its hydrochloride salt, which can be depicted as follows:

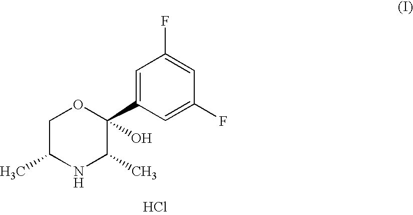

This compound, along with certain pharmaceutical products prepared therefrom, is described in U.S. Pat. No. 5,104,870 (Kelley et al) and noted to be useful in the treatment of depression, anxiety disorders, attention deficit disorders (e.g., ADHD), sexual dysfunctions, headaches including migraine, pain, addiction to (or withdrawal from) cocaine, and addiction to (or withdrawal from) tobacco or other nicotine-containing products,; its use in treating nicotine addiction is described in WO99/25355 (Ascher et al.); oral formulations are described in WO 00/18406 (Balik et al.). The active agent GW320659, as well as its hydrochloride salt, is known and can be prepared by known techniques, as described in U.S. Pat. No. 5,104,870 (Kelley et al.).

GW320659 (also referred to as BW1555U88) is a selective neuronal catecholamine reuptake inhibitor. Kelley et al., reported GW320659 to be a potent, selective inhibitor of norepinephrine uptake, with weaker reuptake inhibition effects on dopamine reuptake. Kelley et al., J. Med. Chem. 39:347 (1996).

GW353162

The morpholinol compound of formula (II), (+)-(2S, 3S)-2-(3-chlorophenyl)-3,5,5-trimethyl-2-morpholinol (GW353162), or pharmaceutically acceptable salts and solvates thereof, is disclosed as useful in the treatment of obesity, depression, attention deficit hyperactivity disorder (ADHD), migraine, pain, sexual dysfunction, Parkinson Disease, Alzheimer Disease, addiction to (or withdrawal from) cocaine, and addiction to (or withdrawal from) tobacco or other nicotine-containing products, in WO9937305. GW353162 is a norepinephrine and dopamine reuptake inhibitor useful in the methods of the present invention.

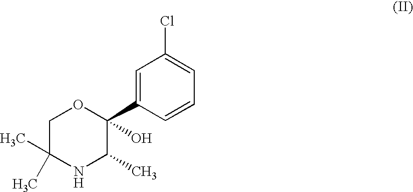

Both GW320659 and GW353162 are analogs of the neuronal monoamine reuptake inhibitor bupropion, known for its use as an antidepressant (2001 Physicians Desk Reference, see also U.S. Pat. Nos. 3,819,706 and 3,885,046).

Other Pharmaceutical Compounds

Monoamines that are widely distributed in the central nervous system include serotonin (an indolamine), and norepinephrine and dopamine (both catecholamines). These compounds are released from the presynaptic space and act as neurotransmitters on presynaptic and postsynaptic receptors. Released neurotransmitters are subject to reuptake into the presynaptic neuron by plasma membrane transporter proteins, where they may be metabolized by the enzyme monoamine oxidase (MAO). MAO type A (MAOA) preferentially deaminates serotonin and norepinephrine, whereas MAO type B (MAOB) deaminates dopamine.

Various chemical compounds and pharmaceutical agents are known that act as neuronal monamine reuptake inhibitors, including those that act as a selective serotonin reuptake inhibitor (SSRI; e.g., fluoxetine, sertraline, paroxetine, fluvoxamine, citalopram, femoxetine); dual serotonin and norepinephrine reuptake inhibitor (SNRI; e.g., duloxetine, medium to high dose venlafaxine); serotonin-2 antagonist/reuptake inhibitor (SARI; e.g., nefazodone); dual norepinephrine and dopamine reuptake inhibitor (NDRI; e.g., bupropion); and norepinephrine reuptake inhibitor (e.g., nisoxetine; LY368975 ((R)-thionisoxetine), Gehlert et al., J. Pharmacol. Exp. Ther. 287:122 (1998)). Additionally, compounds and pharmaceutical agents are known that inhibit the MAO enzymes, including those that are selective for MAOB (e.g., deprenyl) or act as a reversible MAOA inhibitor (e.g., moclobemide). The present methods may be used with any such monoamine reuptake inhibitor.

The present inventors determined that the genetic polymorphisms identified herein are associated with differences in phenotypic response to treatment with the neuronal monoamine reuptake inhibitor GW320659. In the present study, the genotyped subjects had been recruited from a randomized placebo-controlled study of GW320659 for the treatment of obesity, in conjunction with a mildly hypocaloric diet and brief weight management guidance. Subjects had been randomized into one of five treatment groups: placebo or GW320659 at 2.5 mg/day, 5 mg/day, 10 mg/day, or 15 mg/day; outcome measurements included weight loss over baseline weight and changes in supine heart rate, supine diastolic blood pressure, and supine systolic blood pressure.

The primary outcome measurement was the individual's absolute change from baseline body weight (weight at week 0, randomization visit) to weight after 24 weeks of treatment. Genotypes associated with at least a 5.8 kg average weight loss over the 24 week study for subjects receiving 15 mg/day GW320659 are shown in Table 2. The average weight loss in the total (non-genotyped) clinical trial of GW320659 (at the 15 mg/day dosage) was 3.7 Kg (data not shown).

TABLE 2

| | | | Average Weight Loss |
|---|---|---|---|
| Polymorphism | Genotype | Frequency | (Kg)(range) |
| NET1 G155A | 2,2 | 4/37 (11%) | −9.0 (−20.4, −3.3) |
| NET1 T342C | 2,2 | 7/37 (19%) | −7.1 (−13.7, 0) |
| NET1 C120A | 2,2 | 18/36 (50%) | −5.8 (−20.4, 1.3) |
| DAT1 | 10,9 | 10/32 (31%) | −6.6 (−20.4, 1.3) |
| DAT1 | 9,9 | 4/32 (13%) | −7.6 (−22.1, −1.1) |
| NR1 G1001C | 1,2 | 3/37 (8%) | −6.0 (−13.7, −0.9) |
| NR1 G6435A | 2,2 | 4/37 (11%) | −6.9 (−9.2, −3.3) |
| 5HTT G769T | 1,1 | 5/36 (14%) | −6.0 (−9.5, −3.2) |
| 5HTT G160A | 2,2 | 6/37 (16%) | −6.1 (−10.3, −3.2) |

Additionally, the 5HTT Del-Ins (1,2) genotype was associated with an average -5.2 Kg weight loss (15 mg/day dose). FIG. 8.

Further, it was found that NET1 C120A, NET1 G155A, and NET1 T342C were in linkage disequilibrium.

Accordingly, a method of assessing an individual's likelihood of achieving an increased weight loss when treated with a neuronal monoamine reuptake inhibitor involves genotyping one or more polymorphic loci in the above-noted genes, to determine whether the individual has a genotype that has been associated with increased weight loss (increased relative to the weight loss experienced by a treated population that has not been divided by genotype, or relative to individuals with alternate genotypes at the target polymorphic loci).

Outcome measures in addition to weight loss were assessed in the present study, including change in supine diastolic and systolic blood pressure (DBP and SBP), and change in supine heart rate (HR). The present results indicate that changes in heart rate and blood pressure are associated with the MAOB G644A and the DRD2 C12121T polymorphisms.

The present results indicate that increased elevations in heart rate and DBP during treatment were associated with the DRD2 C12121T (2,2) genotype. (See FIGS. 12 & 13, 27 & 28).

Further, increased elevations in DBP during treatment was associated with the occurrence of the MAOB G644A (1,1 and 1,2) genotypes. (FIGS. 18 & 24).

Accordingly, a method of assessing an individual's likelihood of experiencing an increased change in blood pressure (diastolic or systolic) or heart rate when treated with a neuronal monoamine reuptake inhibitor involves genotyping polymorphic loci in the above-noted genes, to determine whether the individual has a genotype that has been associated with increased HR or blood pressure changes (increased relative to the changes in blood pressure or HR experienced by a treated population that has not been divided by genotype, or relative to individuals with alternate genotypes at the target polymorphic loci).

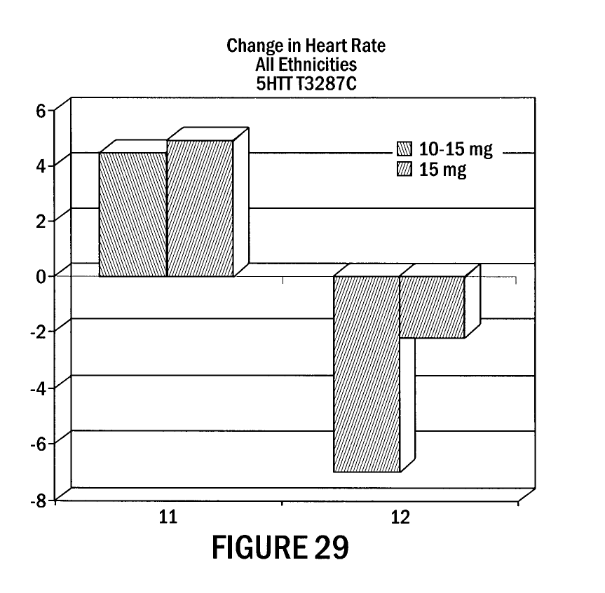

The present results indicate that decreased elevations in heart rate during treatment (10 mg/day+15 mg/day dosage) was associated with the occurrence of the 5HTT T3287C (1,2) genotype, compared to the (1,1) genotype. FIG. 29.

Using a haplotype analysis, the present results indicate that three markers in 5HTT are significantly associated with change in heart rate (5HTT del-ins; 5HTTC867T; and 5HTT T3287C). 5HTT del-ins and 5HTT C867T were found to be in complete linkage disequilibrium, while 5HTT T3287C was in moderate linkage disequilibrium with these two markers.

Accordingly, a method of assessing an individual's likelihood of experiencing an increased elevation in heart rate when treated with a neuronal monoamine reuptake inhibitor involves genotyping polymorphic loci in the above-noted genes, to determine whether the individual has a genotype that has been associated with increased changes (increased relative to the changes in heart rate experienced by a treated population that has not been divided by genotype, or relative to individuals with alternate genotypes at the target polymorphic loci).

The present studies further examined the phenotypic responses of subgroups defined by a multi-locus genotype. As shown in FIG. 18, the group of subjects (10 mg/day or 15 mg/day of GW320659) with the DRD2 C12121T (2′,2) and MAOBG644A (1,1) genotypes, displayed a larger increase in heart rate compared to that in subjects with alternate genotypes. Accordingly, the methods of the present invention further include genotyping subjects at multiple polymorphic sites, to identify subjects having genotypes associated with undesirable side effects.

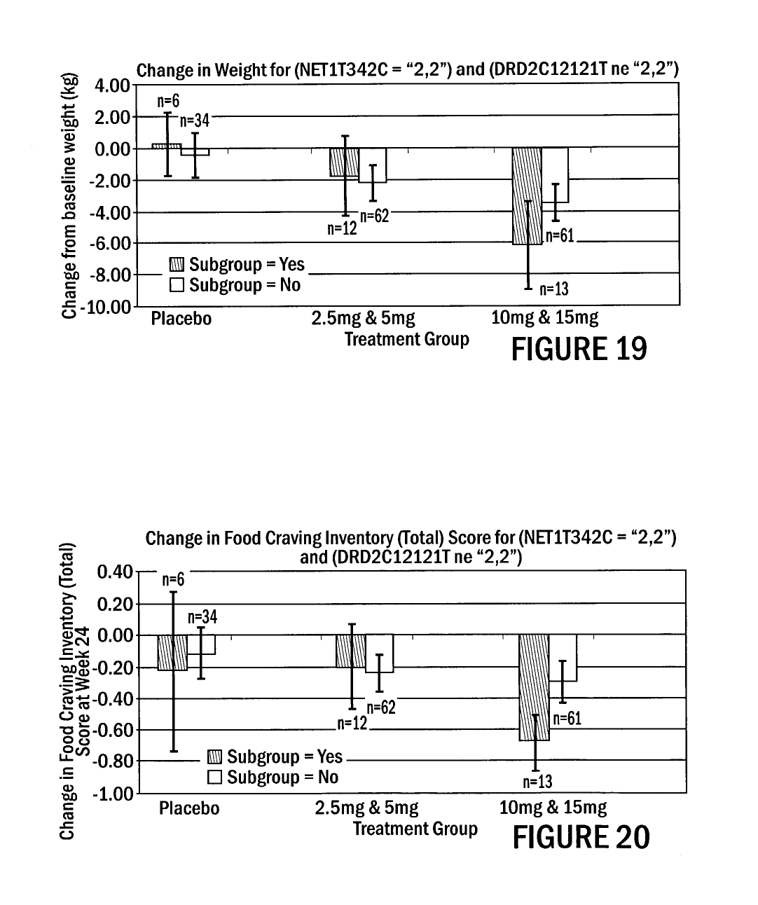

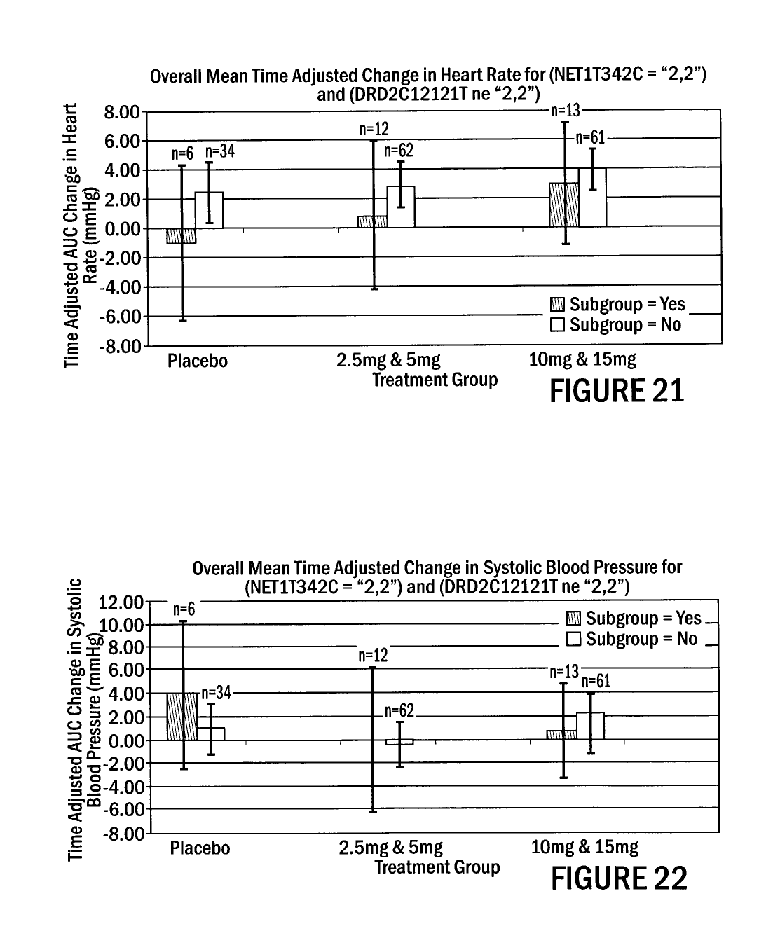

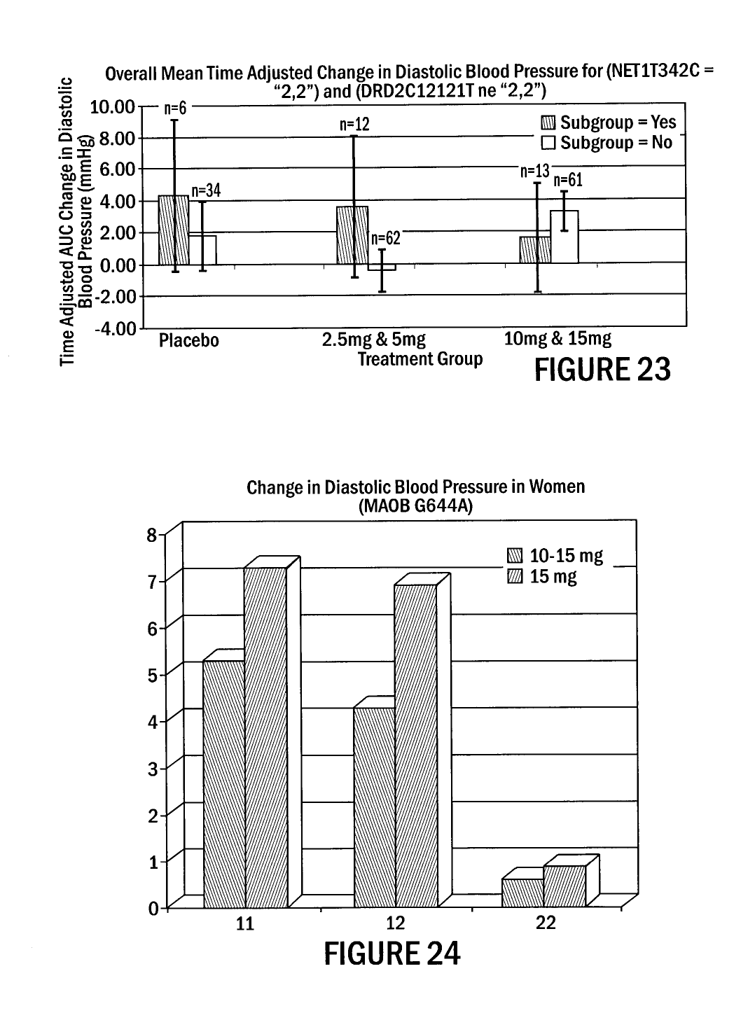

The methods of the present invention further comprise genotyping a subject at a polymorphic locus associated with increased weight gain, and at a polymorphic locus associated with the occurrence of a side effect. The group of subjects (10 mg/day or 15 mg/day of GW320659) with the NET1 T342C (2,2) genotype (associated with increased weight loss) and without the DRD2 C12121T (2,2) genotype (associated with increased heart rate and blood pressure) displayed increased weight loss (FIG. 19) and decreased changes in heart rate and blood pressure (FIGS. 21-23), compared to subjects with alternate genotypes. Accordingly, the methods of the present invention further include genotyping subjects at multiple polymorphic sites, to identify subjects having genotypes associated with both increased weight gain and reduced side effects.

According to the present methods, subjects who are in need of medical treatment with a neuronal monoamine reuptake inhibitor, such as for weight reduction or weight control, can be genetically screened as an aid in predicting their response to such treatment. Treatment preferably utilizes a compound that inhibits norepinephrine reuptake, a compound that inhibits serotonin reuptake, or a compound that inhibits dopamine reuptake. Such compounds include the norepinephrine/dopamine reuptake inhibitor GW320659 (formula I herein) and pharmaceutically acceptable salts thereof, and the morpholinol compound GW353162 (formula II herein), and pharmaceutically acceptable salts thereof.

Screening comprises obtaining a biological sample from the subject and analyzing it to determine the genotype (presence/absence of polymorphic alleles) at a predetermined polymorphic site(s) as specified herein, where different genotypes at that site have previously been associated with different rates of a phenotypic response to pharmaceutical treatment with a neuronal monoamine reuptake inhibitor. More particularly, the pharmaceutical treatment may utilize a norepinephrine reuptake inhibitor, a serotonin reuptake inhibitor, a dopamine reuptake inhibitor, or GW320659; and the treatment may be for obesity, weight reduction or weight maintenance.

The method may include stratifying subjects according to polymorphic sites in several genes, where a particular combination of polymorphic alleles in the genes has been determined to be associated with different rates of a phenotypic response to pharmaceutical treatment with a neuronal monoamine reuptake inhibitor.

The presence of a particular predetermined genotype therefore indicates an increased likelihood that the individual subject will exhibit the associated phenotype. The genotype will rarely be absolutely predictive, i.e., where a population with a certain genotype displays a high incidence of a particular phenotype, not every individual with that genotype will display the phenotype. However, it will be apparent to those skilled in the art that genotyping a subject as described herein will be an aid in predicting the response a subject will have to treatment with a pharmaceutical neuronal monoamine reuptake inhibitor, particularly norepinephrine reuptake inhibitors, and more specifically GW320659. The present methods may further comprise administering a pharmaceutical neuronal monoamine reuptake inhibitor to subjects after screening, in those subjects where the risk of a side effect (e.g., increased heart rate or blood pressure) or the chance of success (e.g., weight loss of a certain amount over a defined time period) is deemed acceptable; the final treatment decision will be based on factors in addition to genetic screening (as will be readily apparent to one skilled in the art), including the subject's overall health status and expected treatment outcome.

In view of the present disclosure, it will be apparent to one skilled in the art how to determine additional NET1, DAT1, NR1, 5HTT, MAOB, and/or DRD2 genotypes that are associated with an increased risk of unacceptable blood pressure or heart rate changes, or increased chance of acceptable weight loss, in response to pharmaceutical treatment with a neuronal monoamine reuptake inhibitor. Various allelic forms of these genes are known, and methods of typing the genes are known in the art. As additional polymorphisms are detected in these genes in humans, typing for such polymorphisms may be based on known methods. Accordingly, one may type a population of subjects who have received a neuronal monoamine reuptake inhibitor and correlate such genotypes with the occurrence of phenotypes as described herein. In an alternate method, one may genotype only those subjects who have experienced a particular phenotypic response and, where the prevalence of a particular allele is known in a general population (i.e., one that has not been subdivided by genotype), determine whether the allele is over-represented in the population displaying the phenotype. As will be apparent to one skilled in the art, the detection of a particular defined polymorphic allele may be accomplished by typing for genetic markers that are known to be in linkage disequilibrium with the target allele/polymorphism.

As multiple NET1, DAT1, NR1, 5HTT, MAOB, and/or DRD2 genotypes exist, the relative incidence of the phenotypic responses described herein may vary among the multiple genotypes. E.g., in a multi-locus screening method where more than two genotypes are found, relative risk may be determined to be highest for one genotype, lowest for another, and intermediate in others. ‘Increased risk’ may be as compared to the risk in a population that has not been stratified by genotype (a general population), or increased as compared to the risk expected in another defined genotype.

Definitions

“Pharmaceutical weight loss treatment” as used herein refers to administration of a pharmaceutical compound to an individual whose weight is greater than a medically acceptable or medically desirable amount, to achieve a reduction in the subject's weight. “Pharmaceutical treatment of obesity” is an aspect of pharmaceutical weight loss treatment and refers to such treatment for individuals whose body mass meets an accepted medical definition of obesity. One commonly accepted measure of overweight is the Body Mass Index (BMI); overweight may be defined as a BMI of at least 25 kg/m², with obesity defined as a BMI of at least 30 kg/m². Pharmaceutical weight loss treatment may be accompanied by a change in diet and/or other behavioral modifications such as support groups and/or patient education. As used herein, pharmaceutical weight loss treatment does not imply a “cure” for obesity or permanent weight loss.

Body Mass Index is a numerical measurement of relative weight for height, and has been significantly correlated with total body fat content. BMI is calculated as weight (kg)/height squared (m²). See, e.g., Clinical Guidelines on the Identification, Evaluation, and Treatment of Overweight and Obesity in Adults. Am. J. Clin. Nutr. 68:899 (1998).

As used herein, “adult subjects” refer to humans over the age of 18 years.

As used herein, “genotyping” a subject (or DNA sample) for a polymorphic allele at a defined genomic locus or “determining the genotype” at a polymorphic allelic site, means detecting which forms of the allele are present in a subject (or a sample). As is well known in the art, an individual may be heterozygous or homozygous for a particular allele. More than two forms of an allele may exist; thus there may be more than three possible genotypes. As used herein, an allele may be ‘detected’ when the other possible allelic variants have been ruled out; i.e., where a specific nucleic acid position is found to be neither adenine (A), thymine (T) or cytosine (C), it can be concluded that guanine (G) is present at that position (G is ‘detected’).

As used herein, “determining” a subject's genotype does not require that a genotyping technique be carried out where a subject has previously been genotyped and the results of the previous genetic test are available; determining a subject's genotype accordingly includes referring to previously completed genetic analyses.

As used herein, a “genetic subset” or “genetic subgroup” of a population consists of those members of the population having a particular genotype. In the case of a biallelic polymorphism, a population can potentially be divided into three subsets: homozygous for allele 1 (1,1), heterozygous (1,2), and homozygous for allele 2 (2,2).

A “population”, as used herein, refers to a group of individuals meeting preselected criteria. A population may refer to a group of individuals having a certain medical condition or disease, those treated with a certain pharmaceutical compound, those of a certain ethnic background, etc. As it is usually not practical to study all individuals meeting a preselected criteria (e.g., all people with a certain medical condition), studies are preferably performed using a population of a limited number of subjects, where that population is considered to be representative of the entire population.

As used herein, a subject that is “predisposed to” or “at increased risk of” a particular phenotypic response based on genotyping of a polymorphic allele will be more likely to display that phenotype than an individual with a different genotype at that polymorphic allele; the difference may be statistically significant. Where the phenotypic response is based on a biallelic or multiallelic polymorphism, the relative risk of a particular response may differ among the multiple possible genotypes.

As used herein, an ‘increased risk’ (or ‘increased incidence’) in a population selected by genotype may be as compared to the risk (or incidence) in a population that has not been stratified by genotype (a general population), or increased as compared to the risk expected in an alternate defined genotype.

As used herein, a pharmaceutical compound for the treatment of obesity or for weight loss treatment is one where administration (in an appropriate pharmaceutical formulation and in a therapeutically effective amount) has been shown to result in or increase weight loss over time (compared to that achieve without the compound), without causing unacceptable side effects. Such therapeutic effectiveness is typically evidenced by Regulatory Authority (eg FDA, EMEA) approval of the pharmaceutical preparation, or by publication of the results of clinical studies in peer-reviewed medical journals. Therapeutically effective amounts of such compounds can be readily determined by those skilled in the art using, e.g., dose-response studies.

As used herein, a “phenotypic response” to pharmaceutical treatment is a measurable response to such treatment. Measurement may be objective (weight loss) or self-reported (hunger). Such phenotypic responses include but are not limited to weight loss of at least a minimum amount over a pre-determined period of time, changes in heart rate, and changes in blood pressure.

As used herein, a “side effect” is an undesirable response to the administration of a pharmaceutical compound, i.e., an effect that is not directed to alleviating the symptoms or the cause of the condition being treated. Side effects range from minor inconveniences to more serious events.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype). The screening and/or selection methods of the present invention may be positive methods, where a subject is selected for treatment based on genotyping results. Alternatively, the screening and/or selection methods according to the present invention may be negative methods, where a subject is eliminated or excluded from treatment based on genotyping results.

“Linkage disequilibrium” refers to the tendency of specific alleles at different genomic locations to occur together more frequently than would be expected by chance. Alleles at given loci are in complete equilibrium if the frequency of any particular set of alleles (or haplotype) is the product of their individual population frequencies A commonly used measure of linkage disequilibrium is r:

$$r = \frac{{\hat{Δ}}_{AB}}{\sqrt{( {\tilde{π}}_{A} + {\hat{D}}_{A} ) ( {\tilde{π}}_{B} + {\hat{D}}_{B} )}}$$

$$where$$

$${\tilde{π}}_{A} = {\tilde{p}}_{A} ( 1 - {\tilde{p}}_{A} ) , {\tilde{π}}_{B} = {\tilde{p}}_{B} ( 1 - {\tilde{p}}_{B} ) , {\hat{D}}_{A} = {\tilde{P}}_{AA} - {\tilde{p}}_{A}^{2} , {\hat{D}}_{B} = {\tilde{P}}_{BB} - {\tilde{p}}_{B}^{2}$$

$${\hat{Δ}}_{AB} = \frac{1}{n} {n}_{AB} - 2 {\tilde{p}}_{A} {\tilde{p}}_{B}$$

nr² has an approximate chi square distribution with 1 degree freedom for biallelic markers. Loci exhibiting an r that corresponds to a significiant chi-squared statistic at the 0.05 level are considered to be in linkage disequilibrium (BS Weir 1996 Genetic Data Analysis II Sinauer Associates, Sunderland, Md.).

As used herein, determination of a ‘multi-locus’ genotype refers to the detection within an individual of the alleles present at more than one locus. For example, a subject may be genetically screened to determine the presence or absence of both a NET1 allele (e.g., the NET1 T342C allele) and a DRD2 allele (e.g., at the DRD2 C12121T locus).

As used herein, the process of detecting an allele or polymorphism includes any suitable method as is known in the art. The allele or polymorphism detected may be functionally involved in affecting an individual's phenotype, or it may be an allele or polymorphism that is in linkage disequilibrium with a functional polymorphism/allele. Polymorphisms/alleles are evidenced in the genomic DNA of a subject, but may also be detectable from RNA, cDNA or protein sequences transcribed or translated from this region, as will be apparent to one skilled in the art.

Alleles, polymorphisms or genetic markers that are ‘associated’ with a phenotypic response to a neuronal monoamine reuptake inhibitor (such as GW320659) are over-represented in subjects displaying that phenotypic response, as compared to subjects who do not display the phenotype, or as compared to the general population.

Treatment of a subject with a pharmaceutical neuronal monoamine reuptake inhibitor comprises administration of an effective amount (for the condition being treated) of the pharmaceutical agent to a subject. The dose of agent is determined according to methods known and accepted in the pharmaceutical arts, and can be determined by those skilled in the art.

Genetic Studies

Genetic association studies show the coexistence of a polymorphism and a phenotype in a population. Association studies are based upon linkage disequilibrium, a phenomenon that occurs between a genetic marker and a phenotype if the marker polymorphism is situated in close proximity to the functional polymorphism. Since the marker and functional polymorphism are in close proximity, it requires many generations of recombination to separate them in a population. Thus they tend to co-exist together on the same chromosome at a higher than expected frequency. A marker is said to be associated with a specific phenotype when its frequency is significantly higher among one phenotype group compared to its frequency in another.

Polymorphisms that are in linkage disequilibrium with each other can be spaced over large regions. Linkage disequilibrium has been reported in regions as small as 1 kilobase or as large as 500 kilobases. Polymorphisms throughout a gene can be in linkage disequilibrium with each other, such that it is valuable to study the whole genome structure—introns, exons, promoters and transcriptional regulatory regions, and 3′ and 5′ untranslated regions. Where a non-functional polymorphism is in linkage disequilibrium with a functional polymorphism that is associated with a particular phenotype, screening for the non-functional polymorphism as well as the functional polymorphism can be used to identify subjects likely to exhibit that phenotype.

The present inventors have determined that polymorphisms in various genes are associated with subjects' phenotypic responses to pharmaceutical treatment with neuronal monoamine reuptake inhibitors; thus genotyping of these genes (either directly or via the gene's expression product) will be useful in identifying therapeutic compounds with measurable effects that vary among subject genotypes. The phenotypic effect to be measured will depend on the particular condition being treated, the therapeutic compound, and the patient population, as will be apparent to one skilled in the art. Where treatment is for weight loss or weight maintenance, desirable phenotypic effects include increased weight loss over time (compared to placebo or an alternate treatment) or decreased desire for food (compared to placebo or an alternate treatment). Measurement may be objective (change in weight) or subjective (e.g., by patient self-reporting).

Accordingly the present methods may be used to select a treatment population of subjects from a larger starting population, to obtain a treatment population having an increased proportion of subjects with favorable genotypes, i.e., genotypes that have been associated with desirable outcomes in response to treatment with GW320659 or GW353162, or with other neuronal catecholamine reuptake inhibitors. Selection of a treatment population having a higher proportion of individuals with favorable genotypes (compared to the starting population) will result in increased efficacy, on average, in the treatment population than would have been achieved in the total starting population. Methods of measuring desirable outcomes (efficacy) will vary depending on the condition being treated, as will be apparent to those skilled in the art. Methods of assessing efficacy of treatments for depression, smoking cessation or nicotine addiction, weight loss treatment, anxiety disorders, attention deficit disorders (e.g., ADHD) and sexual dysfunctions will be apparent to those skilled in the art, e.g., as described in the published medical literature or as used in current clinical trial practice.

Associating a particular genotype with a therapeutic response will assist in determining whether a subsequent individual with that genotype is likely to experience a similar therapeutic response to the same treatment. As used herein, the term polymorphism includes Single Nucleotide Polymorphisms (SNPs), insertion/deletion polymorphisms; microsatellite polymorphisms; and variable number of tandem repeat (VNTR) polymorphisms.

According to the present methods, a neuronal monoamine reuptake inhibitor, such as a norepinephrine or serotonin reuptake inhibitor, may be screened in a population of subjects for variation in its effects, e.g. on weight loss and/or cardiovascular measurements such as blood pressure and heart rate changes. Such methods involve administering the compound to a population, obtaining biological samples from the subjects (which may be done either prior to, during, or after administration of the compound), genotyping polymorphic allelic sites in the genes describe herein, and correlating the genotype of the subjects with their phenotypic responses (both favorable and unfavorable) to the treatment.

Stated another way, the methods of the present invention may be used to determine the correlation of a polymorphic allele with the response of subjects to treatment with a neuronal monoamine reuptake inhibitor (including treatment for weight loss). Subjects in need of treatment are stratified according to genotype for a particular polymorphic allele(s), and their response to a therapeutic agent is assessed (either prospectively or retrospectively) and compared among the genotypes. The response to the therapeutic agent may include either, or both, desired therapeutic responses and undesirable side effects. In this way, genotypes that are associated with an increased (or decreased) rate of therapeutic efficacy, or an increased (or decreased) incidence of a particular side effect, may be identified. The increase or decrease in response is in comparison to the other genotypes, or to a population as a whole. Genetic markers that are found to be associated with (correlated with) the occurrence of a particular phenotype may then be the basis for screening tests to identify subjects most suitable for treatment.

Screening Techniques

Polymorphic alleles may be detected by determining the DNA polynucleotide sequence, or by detecting the corresponding sequence in RNA transcripts from the polymorphic gene, or where the nucleic acid polymorphism results in a change in an encoded protein by detecting such amino acid sequence changes in encoded proteins; using any suitable technique as is known in the art. Polynucleotides utilized for typing are typically genomic DNA, or a polynucleotide fragment derived from a genomic polynucleotide sequence, such as in a library made using genomic material from the individual (e.g. a cDNA library). The polymorphism may be detected in a method that comprises contacting a polynucleotide or protein sample from an individual with a specific binding agent for the polymorphism and determining whether the agent binds to the polynucleotide or protein, where the binding indicates that the polymorphism is present. The binding agent may also bind to flanking nucleotides and amino acids on one or both sides of the polymorphism, for example at least 2, 5, 10, 15 or more flanking nucleotide or amino acids in total or on each side. In the case where the presence of the polymorphism is being determined in a polynucleotide it may be detected in the double stranded form, but is typically detected in the single stranded form.

The binding agent may be a polynucleotide (single or double stranded) typically with a length of at least 10 nucleotides, for example at least 15, 20, 30, or more nucleotides. A polynucleotide agent which is used in the method will generally bind to the polymorphism of interest, and the flanking sequence, in a sequence specific manner (e.g. hybridize in accordance with Watson-Crick base pairing) and thus typically has a sequence which is fully or partially complementary to the sequence of the polymorphism and flanking region. The binding agent may be a molecule that is structurally similar to polynucleotides that comprises units (such as purine or pyrimidine analogs, peptide nucleic acids, or RNA derivatives such as locked nucleic acids (LNA)) able to participate in Watson-Crick base pairing. The agent may be a protein, typically with a length of at least 10 amino acids, such as at least 20, 30, 50, or 100 or more amino acids. The agent may be an antibody (including a fragment of such an antibody that is capable of binding the polymorphism).

In one embodiment of the present methods a binding agent is used as a probe. The probe may be labeled or may be capable of being labeled indirectly. The detection of the label may be used to detect the presence of the probe on (bound to) the polynucleotide or protein of the individual. The binding of the probe to the polynucleotide or protein may be used to immobilize either the probe or the polynucleotide or protein (and thus to separate it from one composition or solution).

In another embodiment of the invention the polynucleotide or protein of the individual is immobilized on a solid support and then contacted with the probe. The presence of the probe immobilized to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism the method may be based on an ELISA system.

The present methods may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

In one embodiment the probe is used in a heteroduplex analysis based system to detect polymorphisms. In such a system when the probe is bound to a polynucleotide sequence containing the polymorphism, it forms a heteroduplex at the site where the polymorphism occurs (i.e. it does not form a double strand structure). Such a heteroduplex structure can be detected by the use of an enzyme that is single or double strand specific. Typically the probe is an RNA probe and the enzyme used is RNAse H that cleaves the heteroduplex region, thus allowing the polymorphism to be detected by means of the detection of the cleavage products.

The method may be based on fluorescent chemical cleavage mismatch analysis which is described for example in PCR Methods and Applications 3:268-71 (1994) and Proc. Natl. Acad. Sci. 85:4397-4401 (1998).

In one embodiment the polynucleotide agent is able to act as a primer for a PCR reaction only if it binds a polynucleotide containing the polymorphism (i.e. a sequence- or allele-specific PCR system). Thus a PCR product will only be produced if the polymorphism is present in the polynucleotide of the individual, and the presence of the polymorphism is determined by the detection of the PCR product. Preferably the region of the primer which is complementary to the polymorphism is at or near the 3′ end the primer. In one embodiment of this system the polynucleotide the agent will bind to the wild-type sequence but will not act as a primer for a PCR reaction.

The method may be a Restriction Fragment Length Polymorphism (RFLP) based system. This can be used if the presence of the polymorphism in the polynucleotide creates or destroys a restriction site that is recognized by a restriction enzyme. Thus treatment of a polynucleotide that has such a polymorphism will lead to different products being produced compared to the corresponding wild-type sequence. Thus the detection of the presence of particular restriction digest products can be used to determine the presence of the polymorphism.

The presence of the polymorphism may be determined based on the change that the presence of the polymorphism makes to the mobility of the polynucleotide or protein during gel electrophoresis. In the case of a polynucleotide single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DGGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the TAQMAN™ PCR detection system. In another method of detecting the polymorphism a polynucleotide comprising the polymorphic region is sequenced across the region which contains the polymorphism to determine the presence of the polymorphism.

Various other detection techniques suitable for use in the present methods will be apparent to those conversant with methods of detecting, identifying, and/or distinguishing polymorphisms. Such detection techniques include but are not limited to direct sequencing, use of “molecular beacons” (oligonucleotide probes that fluoresce upon hybridization, useful in real-time fluorescence PCR; see e.g., Marras et al., Genet Anal 14:151 (1999)); electrochemical detection (reduction or oxidation of DNA bases or sugars; see U.S. Pat. No. 5,871,918 to Thorp et al.); rolling circle amplification (see, e.g., Gusev et al., Am J Pathol 159:63 (2001)); Third Wave Technologies (Madison Wis.) INVADER® non-PCR based detection method (see, e.g., Lieder, Advance for Laboratory Managers, 70 (2000))

Accordingly, any suitable detection technique as is known in the art may be utilized in the present methods

Kits

The present invention also provides for a predictive (patient care) test or test kit. Such a test will aid in the therapeutic use of pharmaceutical neuronal monoamine reuptake inhibitors, including norepinephrine reuptake inhibitors such as GW320659, based on pre-determined associations between genotype and phenotypic response to the therapeutic compound. Such a test may take different formats, including:

(a) a test which analyzes DNA or RNA for the presence of predetermined alleles and/or polymorphisms. An appropriate test kit may include one or more of the following reagents or instruments: an enzyme able to act on a polynucleotide (typically a polymerase or restriction enzyme), suitable buffers for enzyme reagents, PCR primers which bind to regions flanking the polymorphism, a positive or negative control (or both), and a gel electrophoresis apparatus. The product may utilize one of the chip technologies as described by the state of the art. The test kit would include printed or machine readable instructions setting forth the correlation between the presence of a specific genotype and the likelihood that a subject treated with a specific pharmaceutical compound will experience a hypersensitivity reaction;

(b) a test which analyses materials derived from the subject's body, such as proteins or metabolites, that indicate the presence of a pre-determined polymorphism or allele. An appropriate test kit may comprise a molecule, aptamer, peptide or antibody (including an antibody fragment) that specifically binds to a predetermined polymorphic region (or a specific region flanking the polymorphism). The kit may additionally comprise one or more additional reagents or instruments (as are known in the art). The test kit would also include printed or machine-readable instructions setting forth the correlation between the presence of a specific polymorphism or genotype and the likelihood that a subject treated with a specific synthetic nucleoside analog will experience a hypersensitivity reaction.

Suitable biological specimens for testing are those which comprise cells and DNA and include, but are not limited to blood or blood components, dried blood spots, urine, buccal swabs and saliva.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### EXAMPLES

### Example 1

### Clinical Study and Genotyping

Using genotyping methods as are well known in the art, genetic data were obtained from approximately 200 of more than 500 human subjects enrolled in a dose-ranging efficacy and safety study of GW320659 for weight loss (main study group; a randomized, double-blind, placebo controlled, dose-ranging clinical trial); GW320659 was used in conjunction with a mildly hypocaloric diet and brief weight management guidance. Subjects were between the ages of 18-65 years, with a Body Mass Index (BMI) of 30-40, and were not on any psychotropic medications or other anti-obesity medications. Subjects were randomized into one of five treatment groups: placebo or GW320659 at 2.5 mg/day, 5 mg/day, 10 mg/day, or 15 mg/day (oral administration). In the main study group, for the GW320659 15 mg/day dosage, average weight loss was approximately 3.7 kg (108 subjects; data not shown).

Subjects were assessed for various parameters, including absolute change in body weight from week 0 to week 24 of the study; supine heart rate, diastolic blood pressure and systolic blood pressure at multiple predetermined time-points across the study; and change in Food Craving Inventory (Total) score from week 0 to week 24.

Samples of subjects' DNA were genotyped for the presence of the polymorphisms listed in Table 3 below. Due to assay failure (e.g., poor DNA yield, low sample volume), the number of genotype identifications was less than the total number of subjects genotyped.

TABLE 3

| Gene | Polymorphism | Genotypes | Allele |
|---|---|---|---|
| Dopamine | DAT1-VNTR | 3,3; 9,7; 9,9; | [Number = |
| Transporter (DAT1 | | 10,7; 10,8; | number of |
| or SLC6A3) | | 10,9; 10,10; | repeats] |
| | | 11,10 | |
| Dopamine Receptor | DRD2 C20236T | 1,1; 1,2; 2,2 | 1 = C, 2 = T |
| (DRD2) | DRD2 C32806T | 1,1; 1,2; 2,2 | 1 = C, 2 = T |
| | DRD2 C12121T | 1,1; 1,2; 2,2 | 1 = C, 2 = T |
| Norepinephrine | NET1 G155A | 1,1; 1,2; 2,2 | 1 = G, 2 = A |
| Transporter (NET1 | NET1 T342C | 1,1; 1,2; 2,2 | 1 = T, 2 = C |
| or SLC6A2) | NET1 C120A | 1,1; 1,2; 2,2 | 1 = C, 2 = A |
| Monoamine oxidase | MAOB G644A | 1,1; 1,2; 2,2 | 1 = G, 2 = A |
| B (MAOB) | | | |
| Serotonin | 5HTT Del/Ins | 1,1; 1,2; 2,2 | 1 = ins; 2 = del |
| transporter | 5HTT T623C | 1,1; 1,2; 2,2 | 1 = T, 2 = C |
| (5HTT | 5HTT G769T | 1,1; 1,2; 2,2 | 1 = G, 2 = T |
| or SLC6A4) | 5HTT A2631C | 1,1; 1,2; 2,2 | 1 = A, 2 = C |
| | 5HTT C867T | 1,1; 1,2; 2,2 | 1 = C, 2 = T |
| | 5HTT G160A | 1,1; 1,2; 2,2 | 1 = G, 2 = A |
| | 5HTT T3287C | 1,1; 1,2; 2,2 | 1 = T, 2 = C |
| NR1-NMDA | NR1 A1970G | 1,1; 1,2; 2,2 | 1 = A, 2 = G |
| Receptor (NR1 or | NR1 G6435A | 1,1; 1,2; 2,2 | 1 = G, 2 = A |
| GRIN1) | NR1C7701T | 1,1; 1,2; 2,2 | 1 = C, 2 = T |
| | NR1G1001C | 1,1; 1,2; 2,2 | 1 = G, 2 = C |

### Example 2

### Analysis of Clinical Trial Data

Genotypes were parameterized as categorical variables (e.g., 1,1; 1,2; 2,2) and analyses were conducted individually for each of the polymorphic sites noted in Table 3.

Regression Analysis: Changes in Body Weight/Food Craving/Cardiovascular Endpoints

Change in body weight was analyzed using Analysis of Variance (ANOVA); body weight at Week 0 (randomization visit) was used as the baseline weight. The absolute change from baseline in body weight at week 24 was the dependent (response) variable. The primary independent variables were treatment group and genotype. Treatment by genotype interaction was assessed with the appropriate orthogonal trend (treatment) by genotype contrasts. Overall treatment by genotype interaction and pairwise treatment by genotype interaction were assessed and displayed. The primary model is denoted as follows:

Model 1:

Yᵢⱼ=μ+αᵢ+πⱼ+(πα)ᵢⱼ+εᵢⱼ

where

Yᵢⱼ=change from baseline weight at treatment level i and genotype j.

μ=the overall population mean

α₁=the effect of treatment level i

πⱼ=the effect of genotype j

(πα)ᵢⱼ=the interaction effect for genotype j and treatment level i.

εᵢⱼ=the experimental error.

In addition, the dose level for each treatment group was used as a continuous independent (quantitative) predictor. This model was used to analyze weight loss and changes in Food Craving inventory, and was also used to analyze Area Under the Curve (AUC) data for cardiovascular endpoints. The underlying model for this analysis is:

Model 2:

Yⱼ=β₀+β₁Xⱼ₁+β₂Xⱼ₂+β₃Xⱼ₃+β₄Xⱼ₄+β₅Xⱼ₁Xⱼ₂+β₆Xⱼ₁Xⱼ₃+β₇Xⱼ₁Xⱼ₄.+εᵢⱼ

where

Yⱼ=change from baseline weight at genotype j

β₀-β₇=unknown parameters of the model

Xⱼ₁=value for treatment group (0, 2.5, 5, 10, 15)

Xⱼ₂, Xⱼ₃, Xⱼ₄=value for genotype (1, 0)

εᵢⱼ=the experimental error.

No other independent variables were considered for the models.

The endpoint of Change in Food Craving Inventory (total score) was analyzed with the same methods.

Analyses of changes in cardiovascular endpoints (HR, SBP, DBP, all measured in supine subjects) were conducted individually for each of the polymorphic sites, and analyzed as follows. Vital sign assessments at all post-baseline visits were analyzed utilizing a repeated measures (mixed) ANOVA model. Genotype by treatment interactions were assessed. The following model was used.

Model 3:

Yᵢⱼₖₗ=μ+αⱼ+δᵢ+D₍ₖ₎ⱼ+β₁+(αδ)ⱼᵢ+(αβ)₁ⱼ+εᵢⱼₖₗ

where

Yᵢⱼₖₗ=change from baseline weight for the lth time period

on the kth subject in the jth treatment level and ith

genotype

μ=the overall population mean

αⱼ=the effect of treatment level j

δᵢ=the effect of genotype i

D₍ₖ₎ⱼ=the random effects of subjects nested within treatments

β₁=the effects of time

(αβ)₁ⱼ=the interaction effect for time l and treatment level j

(αδ)ⱼ₁=the interaction effect for treatment j and genotype i

εᵢⱼₖ=the experimental error.

If the treatment by time interaction was non-significant, the corresponding term was planned to be removed from the model, prior to assessing treatment by genotype interaction. Subsequently, this term was removed for all vital signs analyses. In addition, vital signs data was analyzed utilizing a time-weighted area under the curve (adjusted for baseline) analysis. Model #2 (used for the efficacy analyses) was employed for these analyses.

Recursive Partitioning

Recursive partioning (HelixTree Software, May 1, 2001) was used to further examine the data. Due to sample size constraints, a combination of automatic and manual splits were used to best describe the underlying relationships between genotypes, treatment dose, and other demographic variables for each selected endpoint. Recursive partioning was applied to the following endpoints: change in weight, change in food craving (total score) inventory, change (Area Under the Curve (AUC) adjusted) in supine heart rate, and change (AUC adjusted) in supine diastolic blood pressure.

Haplotype Analysis

Genotypic data from unrelated individuals do not contain information on which alleles were transmitted from each parent, however haplotype frequencies were estimated using the expectation maximization (EM) algorithm (Demptsrer et al., J Royal Stat Soc B, 1977, 39:1-38). The multilocus genotypes from each individual were used to enumerate all possible haplotypes. These haplotypes were assigned starting frequencies. The haplotype frequencies were updated with frequencies calculated from all the possible haplotypes from each individual in the sample. This continued until the frequencies were constant from iteration to iteration.

Regression-based models were used to relate inferred haplotype probabilities for each individual with the continuous response.

The following model was used for regression

Yᵢⱼₖ=μ+αᵢ+pⱼₖπⱼ+eᵢⱼₖ

Change from baseline weight at treatment level i and inferred haplotype j

μ=Overall population mean

αᵢ=Effect of treatment levelᵢ

pⱼₖ=EM-inferred haplotype probabilities conditional on k-th person genotype

πⱼ=Effect of inferred haplotype i

eᵢⱼₖ=random error.

Linkage Disequilibrium

Linkage Disequilibrium analysis was conducted for genetic markers that were expected to be close together (<100 kb) in the genome, or for allelic loci that are known to be located in the same gene. A measure of association between alleles (LD) at different loci was computed.

The LD between two loci A and B is given by D_{AB}=p_{AB}−p_{A}p_{B}, where p_{A} is the allele frequency of A allele of marker A and p_{B} is the allele frequency of B allele of marker B. A commonly used measure of LD was calculated as follows:

$${r}^{2} = \frac{{\hat{Δ}}_{AB}^{2}}{\sqrt{( {\tilde{π}}_{A} + {\hat{D}}_{A} ) ( {\tilde{π}}_{B} + {\hat{D}}_{B} )}}$$

$$Where \text{:}$$

$${\tilde{π}}_{A} = {\tilde{p}}_{A} ( 1 - {\tilde{p}}_{A} ) , {\tilde{π}}_{B} = {\tilde{p}}_{B} ( 1 - {\tilde{p}}_{B} ) , {\hat{D}}_{A} = {\tilde{P}}_{AA} - {\tilde{p}}_{A}^{2} , {\hat{D}}_{B} = {\tilde{P}}_{BB} - {\tilde{p}}_{B}^{2}$$

$${\hat{Δ}}_{AB} = \frac{1}{n} {n}_{AB} - 2 {\tilde{p}}_{A} {\tilde{p}}_{B}$$

Markers that were in LD were treated as correlated variables.

### Example 3

### Results: Change in Body Weight

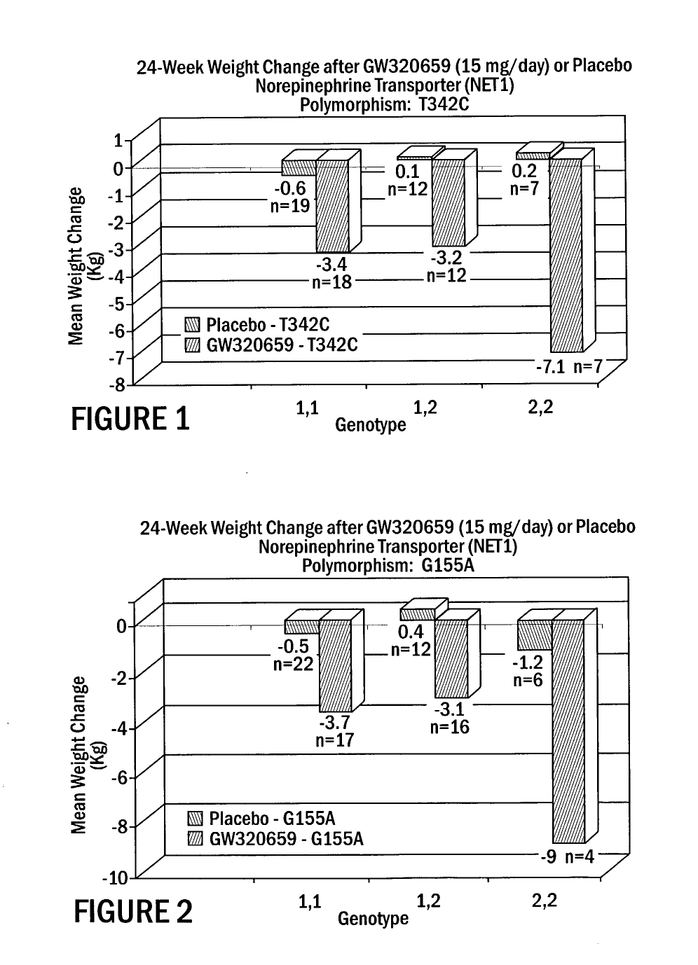

The range of mean absolute change in weight by sub-group (n≧5 subjects per sub-group) varied from 1.5 kg (n=14) for the DRD2 C20236R genetic marker (1,1 genotype, placebo dose) to −7.1 kg (n=7) for the NET1 T342C genetic marker (2,2 genotype, 15 mg. dose) (FIG. 1). The largest absolute change in weight for any sub-group was −9.0 kg (n=4) for the NET1 G155A genetic marker (2,2 genotype, 15 mg. dose) (FIG. 2).

The genetic marker and genotype (n≧5 subjects per sub-group) with the largest placebo adjusted mean absolute change in weight (−7.3 kg) for the 15 mg dose group was the 2,2 genotype for NET1 T342C.

The placebo adjusted mean absolute changes in body weight (for the 15 mg dose group) for the NET1 T342C marker, by genotype, were −2.8 kg (1,1), −3.3 kg (1,2) and −7.3 kg (2,2). The overall level of statistical significance (comparing the slopes of the dose response curve for each genotype) was 0.139 (model 1) and 0.138 (model 2). However, p-values from the pairwise comparison of the dose response slopes of genotypes 1,2 versus 2,2 were 0.062 (model 1) and 0.058 (model 2).

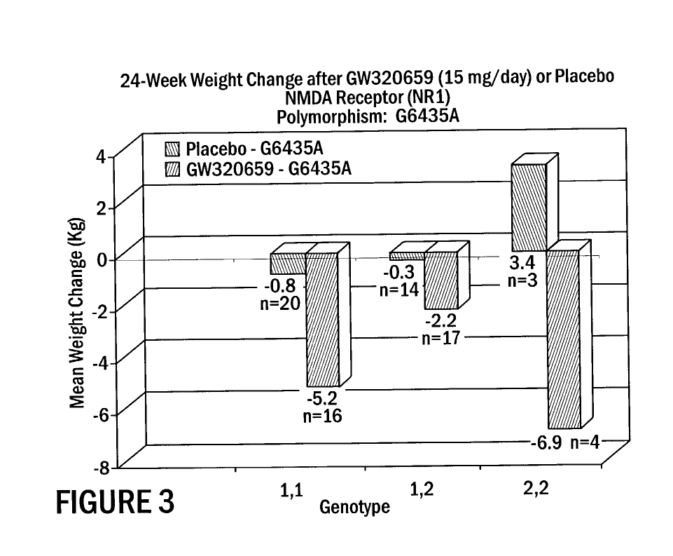

The absolute change in weight for the NR1 G6435A polymorphism (15 mg/d dose) is shown in FIG. 3. The placebo adjusted mean absolute changes in weight (for the 15 mg dose group) for the NR1 G6435A marker by genotype were −4.4 kg (1,1), −1.9 kg (1,2) and −10.3 kg (2,2) (however, there were only three subjects per treatment group for the 2,2 genotype for placebo and four subjects for the GW320659). The overall level of statistical significance (comparing the slopes of the dose response curve for each genotype) was 0.112 (model 1) and 0.096 (model 2). However, p-values from the pairwise comparison of the dose response slopes of genotypes 1,2 versus 2,2 were 0.041 (model 1) and 0.037 (model 2).

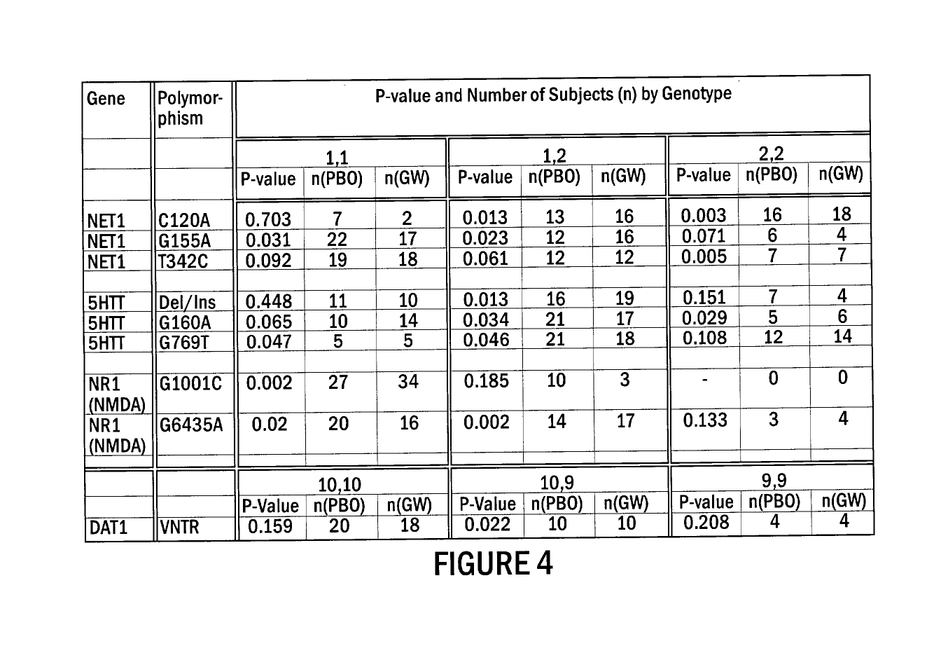

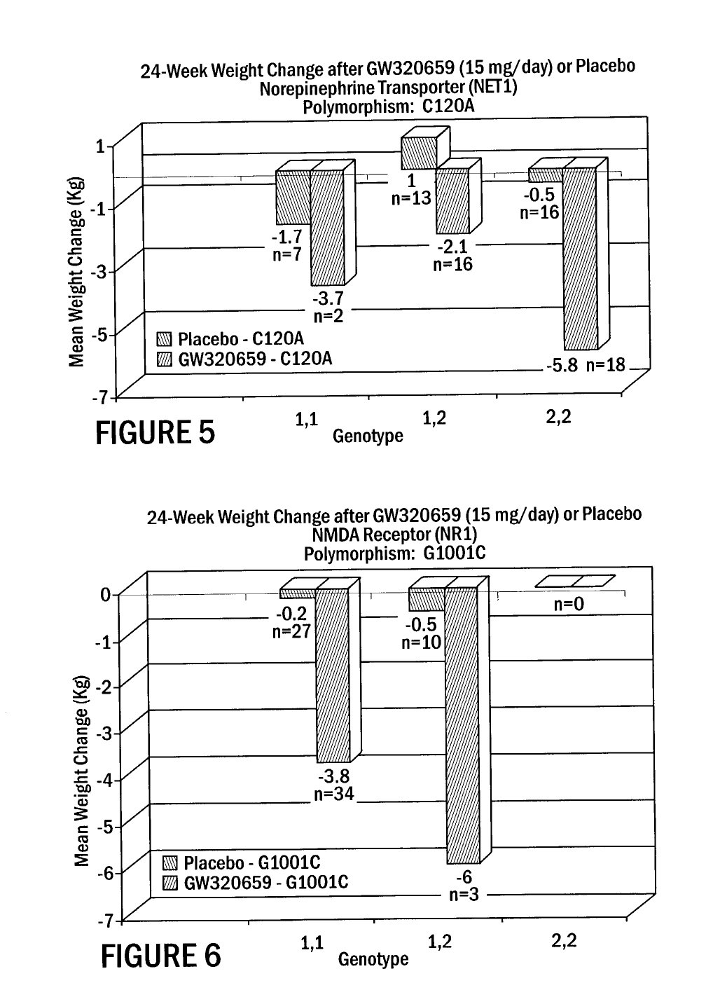

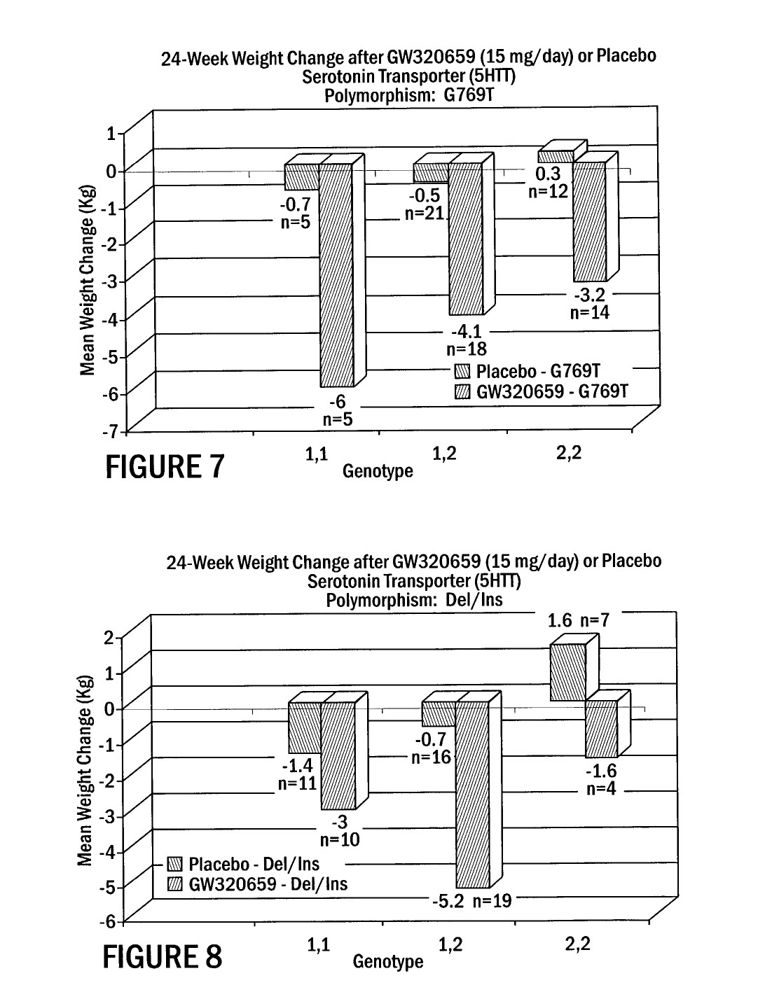

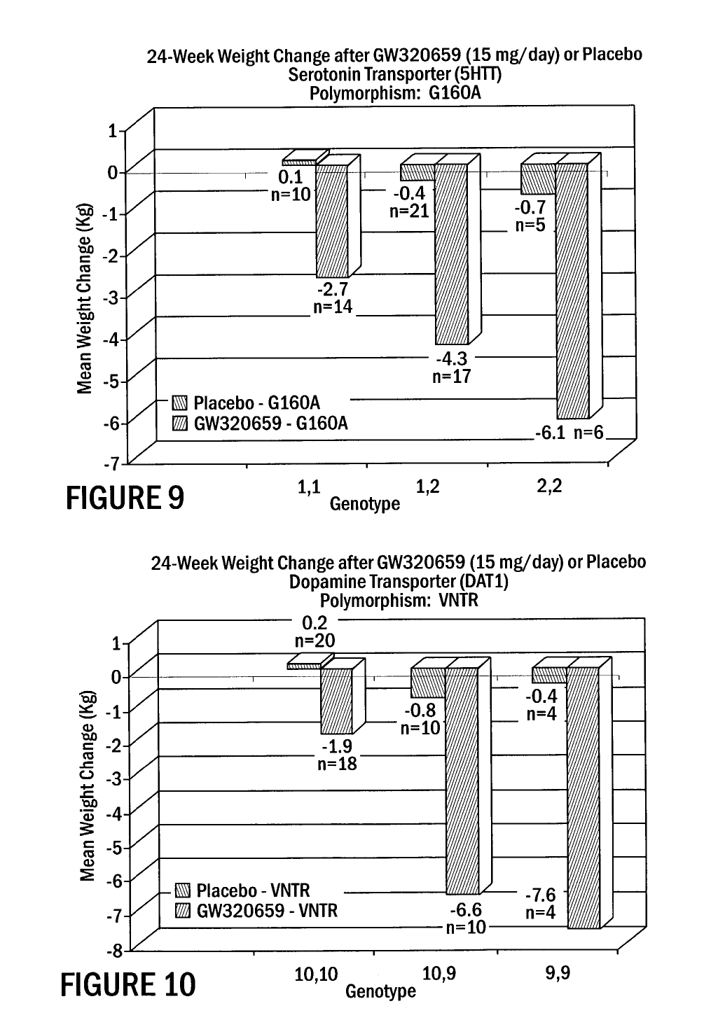

The signficance of weight loss differences between placebo or GW 320659 (15 mg/d dose) after 24 weeks of treatment, is shown in FIG. 4. Mean absolute change in weight for other genotypes (15 mg/day dose) is shown in FIGS. 5-10.

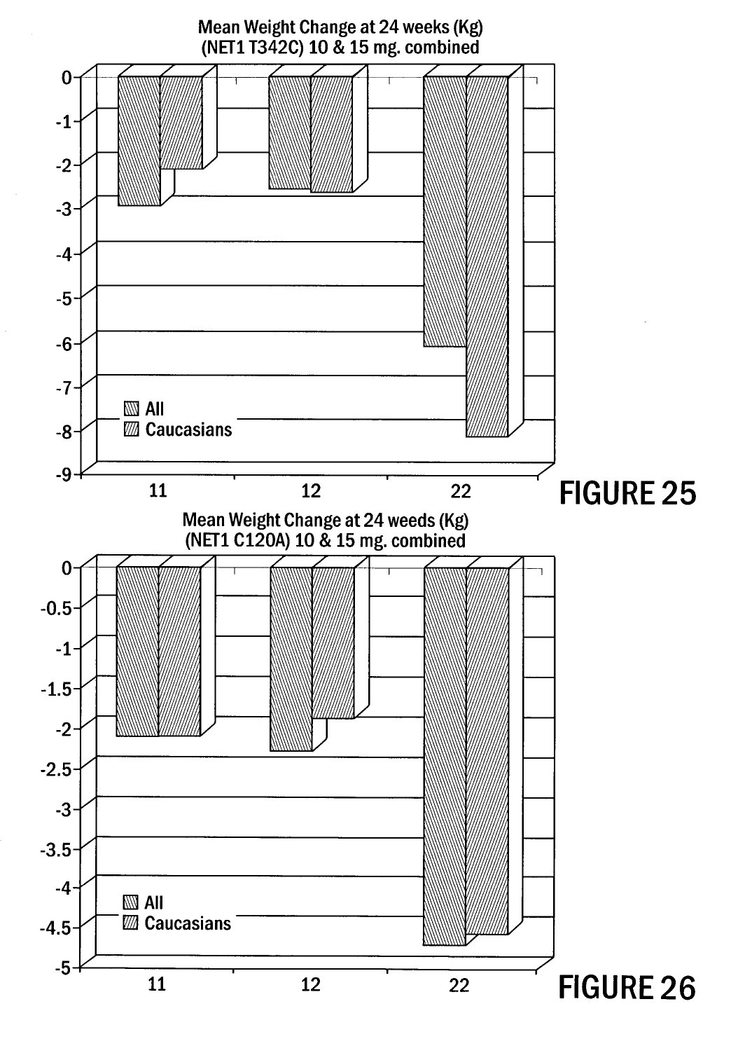

FIGS. 25 & 26 show the mean weight change at 24 weeks for the NET1T342C and NET1C120A polymorphisms, for the combination of dosage groups 10 mg/day and 15 mg/day, for all subjects and for Caucasian subjects.

### Example 4

### Results: Change in Food Craving Inventory

The Food Craving Inventory used was a validated scale developed at Louisiana State University. It is a scale of 37 items designed to measure food cravings for specific foods. For each item the subject is asked “Over the past month, how often have you experienced a craving for the food?”. Possible responses were Never, Rarely, Sometimes, Often, and Always. These responses were converted to ordinal scores (1, 2, 3, 4, 5). The mean total score was computed for each patient. Change from baseline mean scores were analyzed. Possible range for this measure was −4 to 4; a negative number indicates a decrease in cravings.

The range of mean absolute change in Food Craving Inventory (Total) score by sub-group (n≧5 subjects for each sub-group) varied. The genetic marker and genotype (n≧5 subjects per sub-group) with the largest placebo adjusted mean change in Food Craving Inventory (Total) score (0.7) for the 15 mg dose group was the 2,2 genotype for 5HTT G160A. The mean change was −0.7 for the genetic markers DRD2 C20236T (2,2 genotype, 15 mg dose), DRD2 C12121T (1,1 genotype, 15 mg dose) and NET1 T342C (2,2 genotype, 10 mg dose). (Data not shown).

The placebo adjusted mean changes in Food Craving Inventory (Total) score (for the 15 mg dose group) for the NET1 C120A marker by genotype were 0.2 (1,1); −0.4 (1,2); and −0.4 (2,2). The overall level of statistical significance (comparing the slopes of the dose response curve for each genotype) was 0.094 (model 1) and 0.070 (model 2). However, p-values from the pairwise comparison of the dose response slopes of genotypes 1,1 versus 1,2 were 0.050 (model 1) and 0.037 (model 2). P-values from the pairwise comparison of the dose response slopes of genotypes 1,1 versus 2,2 were 0.031 (model 1) and 0.023 (model 2).

### Example 5

### Results: Cardiovascular Measurements

Supine heart rate (HR), supine systolic blood pressure (SBP) and supine diastolic blood pressure (DBP) were assessed at various fixed intervals across the duration of the study. Summaries and analyses were conducted on the “area under the curve” (AUC) values (adjusted for time on study and baseline). All reported p-values in this section refer to AUC analyses, using Model No.2 as described above.

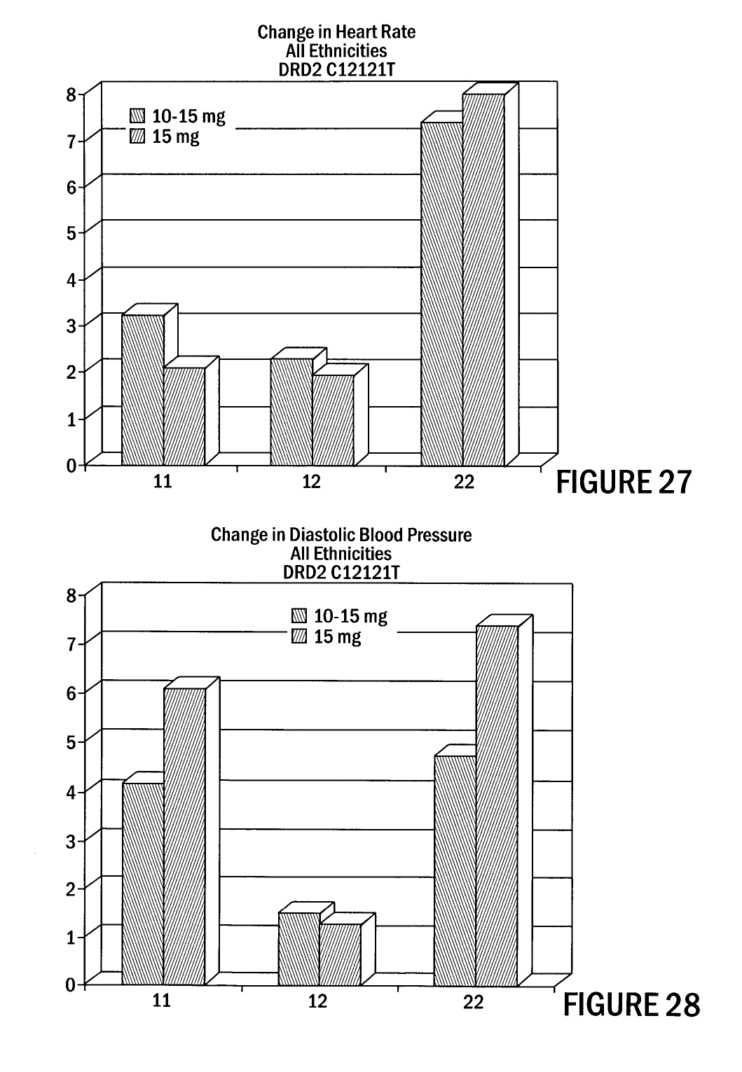

The DRD2 C12121T genetic marker showed a reasonably consistent pattern for the cardiovascular measurements assessed. The mean adjusted AUC changes in supine heart rate, for the 1,1 genotype in the placebo and 15 mg treatment groups, were 0.0 (placebo) and 2.1 (15 mg); for the 1,2 genotype it was 2.3 (placebo) and 1.9 (15 mg); for the 2,2 genotype it was 3.4 (placebo) and 8.0 (15 mg). FIG. 27 shows the change in heart rate for combined (10+15 mg) group and the 15 mg dosage group, for the DRD2 C12121T alleles.

The p-value for the DRD2 C1212T 1,2 versus 2,2 pairwise comparison (of dose response slopes) was 0.117.

The mean adjusted AUC changes in supine systolic blood pressure, by DRD2 C12121T genotype for the 1,1 genotype in the placebo and 15 mg treatment groups were -1.2 (placebo) and 7.9 (15 mg); for the 1,2 genotype it was 3.5 (placebo) and 0.2 (15 mg); and for the 2,2 genotype it was 0.9 (placebo) and 6.1 (15 mg). The p-value from the overall comparison of dose response slopes was 0.006. Both the DRD2 C12121T 1,1 and 2,2 genotypes appeared to have greater positive dose response relationships than the 1,2 genotype (p=0.004 and p=0.018 respectively).

The mean adjusted AUC changes in supine diastolic blood pressure (DBP), by DRD2 C12121T genotype for the 1,1 genotype in placebo and 15 mg treatment groups were −1.6 (placebo) and 6.1 (15 mg); for the 1,2 genotype it was 3.1 (placebo) and 1.3 (15 mg); for the 2,2 genotype it was 4.1 (placebo) and 7.4 (15 mg). The p-value from the overall comparison of dose response slopes was 0.034. Both the 1,1 and 2,2 genotypes appeared to have greater dose response relationships than the 1,2 genotype (p=0.019 and p=0.064 respectively). The trends were very similar for the 10+15 mg/day combined group. The 2,2 genotype of by DRD2 C12121T was associated with both increase in DBP and also heart rate. The MAOB G644A was associated with DBP. FIG. 28 shows the change in DBP for the combined (10+15 mg) group and the 15 mg dosage group, for the DRD2 C12121T alleles.

The mean adjusted AUC changes in HR by 5HTT T3287C genotype for the 1,1 and 1,2 genotypes (10 mg+15 mg dosage group) is shown in FIG. 29.

### Example 6

### Recursive Partitioning Analysis: Food Craving Inventory Scores

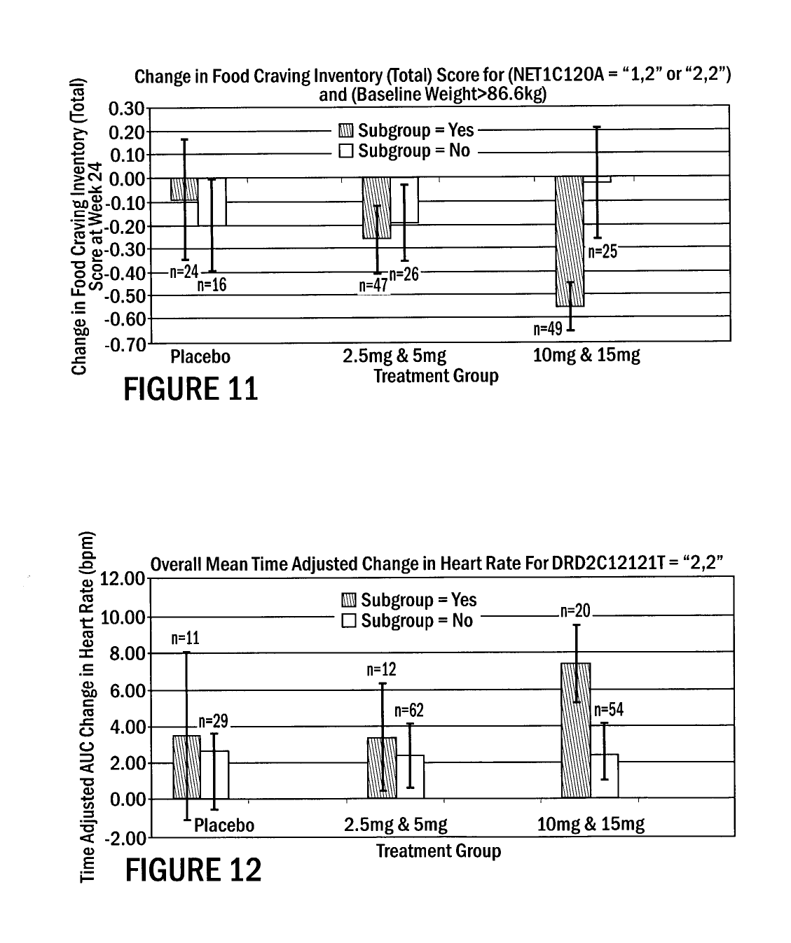

The change in Food Craving Inventory was assessed by grouping low dose treatments together (2.5+5 mg treatments) and higher dose treatments together (10 mg+15 mg), and comparing to placebo, using a p-value threshold set at 0.35 (the minimum level necessary to get further automatic splits). The software then determined the remaining splits for the 10 mg+15 mg group. The splits found in the 10+15 mg group were then manually duplicated for the lower dose group (2.5+5 mg) for comparison purposes. As an example, for the 10 mg+15 mg treatment doses, the mean response for subjects with genotype 1, 2 or 2,2 for the NET1 C120A genetic marker and a baseline weight greater than 86.6 kg, was −0.55 (std=0.37 n=49). FIG. 11. For all subjects not in the defined subgroup, mean response was −0.02 (std=0.60, n=25).

### Example 7

### Recursive Partitioning Analysis: Cardiovascular Measurements

As in Food Craving Inventory (Example 6, above), manual splits were created by treatment dose (placebo, 2.5+5 mg, and 10+15 mg). The p-value threshold was then set at 0.10 (the minimum level necessary to get further automatic splits). The software then determined the remaining splits for the 10+15 mg group. The splits found in the 10+15 mg group were then manually duplicated for the lower dose groups (for comparison purposes).

FIG. 12 shows the overall mean time adjusted change in heart rate for the subgroup of subjects with the 2,2 genotype for DRD2C12121T (where “Subgroup=Yes” indicates the subject met the criteria defined by the recursive partioning analysis).

Results from a clinical trial of GW320659 for the medical treatment of obesity indicated that the 15 mg dose seemed to differentiate from the remaining dose groups (data not shown). Accordingly, for some recursive partioning analysis the treatment groups were split in this manner (15 mg versus all other groups). The p-value threshold was set 0.10 (the minimum level necessary to get further automatic splits). The software then determined the remaining splits for the 15 mg group. The splits found in the 15 mg group were then manually duplicated for the lower dose groups (for comparison purposes).

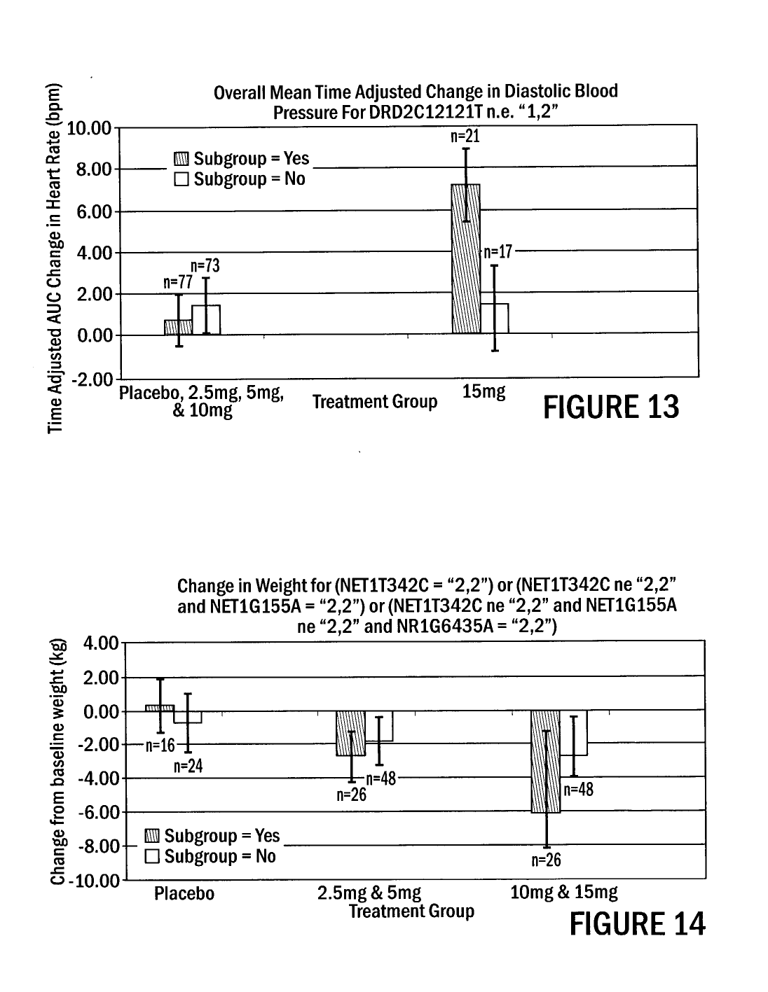

FIG. 13 shows the overall mean time-adjusted change in Diastolic Blood Pressure for the subgroup of subjects who were not 1,2 at DRD2C12121T (i.e., who were 1, 1 or 2,2 at this loci), comparing subjects in the combined (placebo +2.5 mg/day+5.0 mg/day+10 mg/day) dosage group to subjects in the 15 mg/day dosage group.

### Example 8

### Change in Diastolic Blood Pressure: MAOBG644A

FIG. 24 shows the change in DBP for women based on genotype at the MAOBG644A polymorphic site. FIG. 24 compares, for each genotype, the combined (10 mg/day+15 mg/day) dosage group to the 15 mg/day dosage group. Subjects with either the 1, 1 or 1,2 genotype showed greater increases in DBP that subjects with the 2,2 genotype.

### Example 9

### Haplotype Analysis

Haplotype analysis results are shown in Tables 4-11.

TABLE 4

Weight Change for all ethnicities: 10-15 mg - NET1
NET1T342C: 0.0585952
NET1C120A: 0.0400765
NET1G155A NET1T342C: 0.0213925
NET1T342C NET1C120A: 0.11349

TABLE 5

Weight Change, Caucasian: 10-15 mg - NET1
NET1G155A: 0.70549
NET1T342C: 0.00393077
NET1C120A: 0.0309803
NET1G155A NET1T342C: 0.00376779
NET1T342C NET1C120A: 0.00814965
NET1G155A NET1T342C NET1C120A: 0.0131224

TABLE 6

Heart Rate: 10 and 15 mg combined; all ethnicities - 5HTT
5HTTDel-Ins: 0.0323717
5HTTC867T: 0.0266231
5HTTT3287C: 0.0113164
5HTTDel-Ins 5HTTC867T: 0.0336222
5HTTC867T 5HTTT3287C: 0.00814582
5HTTDel-Ins 5HTTC867T 5HTTT3287C: 0.00383495

TABLE 7

Heart Rate: 10 and 15 mg combined; Caucasians - 5HTT
5HTTDel-Ins: 0.124989
5HTTC867T: 0.0197806
5HTTT3287C: 0.00634278
5HTTDel-Ins 5HTTC867T: 0.108958
5HTTC867T 5HTTT3287C: 0.00376723
5HTTDel-Ins 5HTTC867T 5HTTT3287C: 0.00770418

TABLE 8
Weight Loss at 24 weeks
NET1G155A NET1T342C

| Hap- | | | | | | |
|---|---|---|---|---|---|---|
| lo- | All Ethnicities | | | Caucasians | | |
| type | Freq. | Mean | p Value | Freq. | Mean | p Value |
| 1,1 | 0.345 | −2.00695 | 0.006194 | 0.393071 | −1.73903 | 0.005363 |
| 1,2 | 0.391 | −4.32461 | 0.102643 | 0.292114 | −4.41636 | 0.013327 |
| 2,1 | 0.255 | −3.88563 | 0.560114 | 0.301373 | −2.9211 | 0.921295 |
| 2,2 | 0.009 | −9.98991 | 0.066326 | 0.013441 | −9.41331 | 0.014384 |
| Over- | | | 0.021393 | | | 0.003768 |
| all | | | | | | |
| p- | | | | | | |
| value | | | | | | |

TABLE 9
Change in Heart Rate
5HTTDel-Ins 5HTTC867T

| | All Ethnicities | | | Caucasians | | |
|---|---|---|---|---|---|---|
| Haplotype | Freq. | Mean | p Value | Freq. | Mean | p Value |
| 1,1 | 0.375371 | 4.19982 | 0.930015 | 0.332504 | 4.07665 | 0.496341 |
| 1,2 | 0.241816 | 6.42444 | 0.027802 | 0.292496 | 6.80667 | 0.025408 |
| 2,1 | 0.319941 | 2.18331 | 0.007361 | 0.302912 | 3.03449 | 0.046289 |
| 2,2 | 0.062871 | 4.87864 | 0.670641 | 0.072088 | 5.08531 | 0.802465 |
| Overall | | | 0.0335 | | | 0.10896 |
| p-value | | | | | | |

TABLE 10
Change in Heart Rate
5HTTC867T 5HTTT3287C

| | All Ethnicities | | | Caucasians | | |
|---|---|---|---|---|---|---|
| Haplotype | Freq. | Mean | p Value | Freq. | Mean | p Value |
| 1,1 | 0.614294 | 3.5075 | 0.401502 | 0.555563 | 3.81086 | 0.403071 |
| 1,2 | 0.07142 | −0.6851 | 0.011407 | 0.074066 | −0.83466 | 0.005289 |
| 2,1 | 0.314277 | 5.56515 | 0.026623 | 0.370362 | 5.82668 | 0.01978 |
| Overall | | | 0.0081 | | | 0.003767 |
| p-Value | | | | | | |

TABLE 11
Change in Heart Rate
5HTTDel-Ins 5HTTC867T 5HTT3287C

| | All Ethnicities | | | Caucasians | | |
|---|---|---|---|---|---|---|
| Haplotype | Freq. | Mean | p Value | Freq. | Mean | p Value |
| 1,1,1 | 0.290309 | 5.47964 | 0.168503 | 0.243049 | 5.69039 | 0.362671 |
| 1,1,2 | 0.078123 | −0.6849 | 0.008516 | 0.083329 | −0.834462 | 0.002895 |
| 1,2,1 | 0.248755 | 6.40364 | 0.026476 | 0.298622 | 6.80808 | 0.023117 |
| 2,1,1 | 0.326879 | 2.25617 | 0.008897 | 0.309035 | 3.11016 | 0.054991 |
| 2,2,1 | 0.055932 | 4.78231 | 0.737703 | 0.065961 | 4.92013 | 0.883669 |
| Overall | | | 0.0038 | | | 0.007704 |
| p-value | | | | | | |

### Example 10

### Genetically Defined Subgroups: Weight Loss

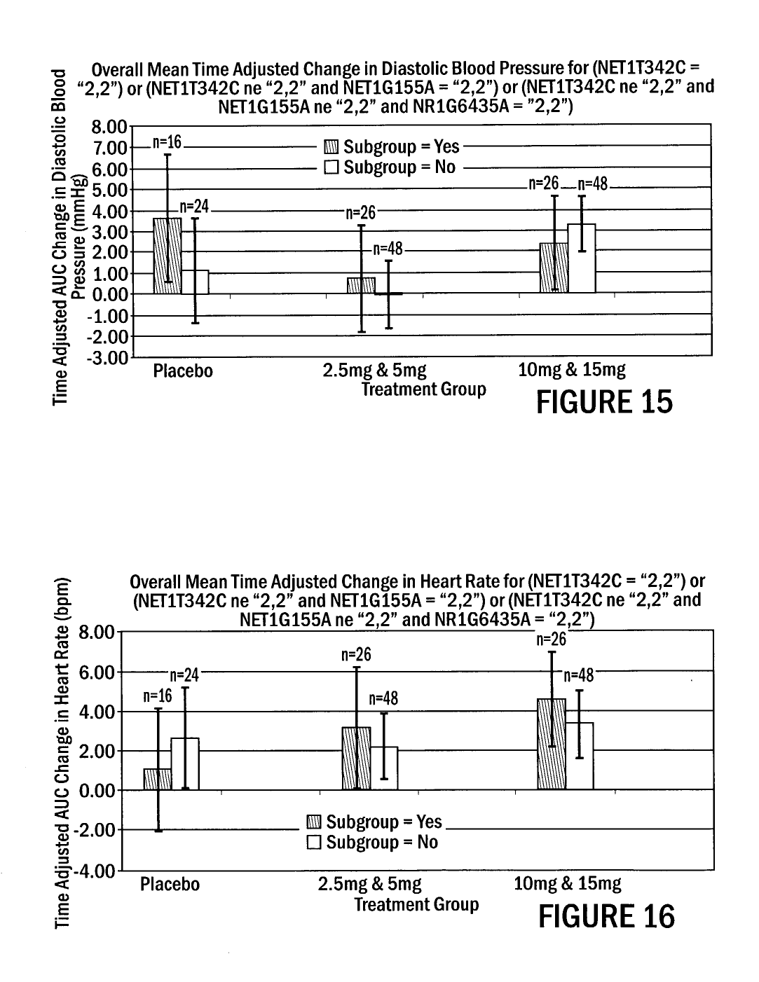

The present study identified a genetically defined subgroup of study participants (individuals who were 2,2 for the marker(s) NET1T342C and/or NET1G155A and/or NR1G6435A) who demonstrated greater weight loss compared to other genetic subgroups. At the 10 mg and 15 mg doses (combined), mean weight loss in this subgroup was 6.05 kg, and was significantly greater than placebo. FIG. 14. This same subgroup did not show a significant difference in diastolic blood pressure (mean rise in DBP 2.4 mmHG for subgroup) compared to that seen in placebo treated individuals; nor was a significant difference seen in heart rate (mean rise in HR 4.7 bpm) compared to that seen in placebo-treated individuals. FIG. 15 and FIG. 16.

Of 74 subjects in the combined (10 mg/day+15 mg/day) dosage group, 26 were members of the subgroup 2/2 NET1T342C and/or 2/2 NET1G155A and/or 2/2 NR1G6435A (described above). The characteristics of this subgroup are shown in Table 12:

| Characteristic | Value |
|---|---|
| Age (years) | 41.34615385 |
| Baseline Weight (kg) | 95.69230769 |
| Change in weight at Week 24 (kg) | −6.05 |
| Mean change in Food Craving Inventory | −0.528 |
| (Total) | |
| Systolic Blood Pressure (mean time | 0.973307692 |
| adjusted change | |
| Diastolic Blood Pressure (mean time | 2.431 |
| adjusted change | |
| Heart Rate (mean time adusted change | 4.665653846 |

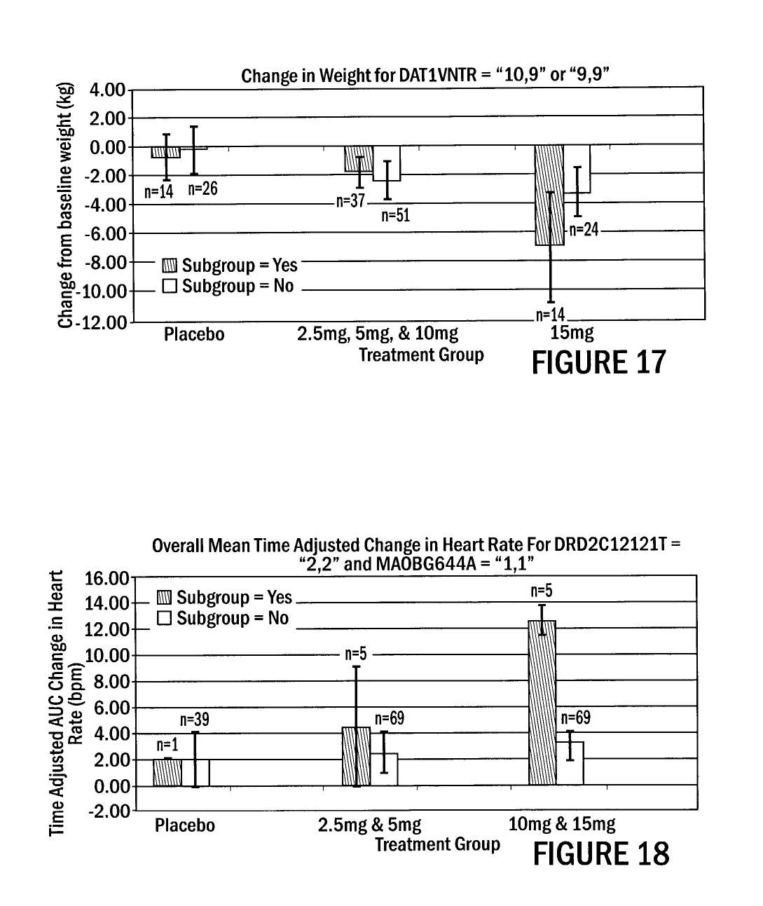

Further, the subgroup of individuals with DAT1 VNTR 9, 9 or 9,10 showed a mean weight loss (15 mg/day) of 6.89 kg. FIG. 17.

Further, a subgroup defined for DRD2 C12121T (1,1) and MAOB G644A (1,1) were identified as showing a distinct change in Heart Rate at 10 mg and 15 mg dosing (mean change in HR 13 bpm). FIG. 18.

### Example 11

### Genetically Defined Subgroup: Weight Loss+Cardiovascular Changes

A desirable phenotypic response to treatment with GW320659 (mean weight loss of at least approximately 6 kg at 24 weeks, with comparatively small changes in HR, SBP and DBP during treatment) was seen in individuals who were 2/2 for NET1T342C (one of the genotypes identified above as associated with increased weight loss) and who were not 2/2 for DRD2C12121T. Results from this subgroup (defined as NET1T342C=2,2 and DRD2C12121T ne 2,2; where “ne” means ‘not equal’) are shown in FIGS. 19-23.

### Example 12

### Screening of Individuals

DNA samples are obtained from a population of subjects in need of treatment for obesity, and genomic DNA is extracted using standard procedures (automated extraction or using kit formats). The genotypes of the subjects, and any control individuals utilized, are determined for polymorphisms within the DRD2, DAT1, NET1, MAOB, 5HTT and/or NR1 gene sequences, using either PCR, PCR-RFLP, TAQMAN™ allelic discrimination assays, or any other suitable technique as is known in the art.

If a specific polymorphism resides in an amplification product that is of sufficient physical size (e.g., an insertion/deletion polymorphism of multiple bases), a simple size discrimination assay can be employed to determine the genotype of an individual. In this case, two primers are employed to specifically amplify the gene of interest in a region surrounding the site of the polymorphism. PCR amplification is carried out, generating products that differ in length, dependent on the genotype (insertion or deletion) they possess. When subjected to gel electrophoresis, the differently sized products are separated, visualized, and the specific genotypes interpreted directly.

PCR-RFLP (polymerase chain reaction—restriction fragment length polymorphism) assays may also be utilized as is known in the art to detect polymorphisms. For each polymorphic site, a PCR-RFLP assay employs two gene-specific primers to anneal to, and specifically amplify a segment of genomic DNA surrounding the polymorphic site of interest. Following PCR amplification, specific restriction endonuclease enzymes are employed to digest the PCR products produced. The enzyme utilized for an assay is selected due to its specific recognition sequence which it requires to bind to, and cleave the PCR product in the presence/absence of the polymorphism, yielding fragments diagnostic of the specific base present at the polymorphic site. Following cleavage by the restriction enzyme, gel electrophoresis is employed to separate and visualize the fragments produced.

TAQMAN™ (PE Applied Biosystems, Foster City, Calif.) assays, as are known in the art, may also be utilized to identify polymorphisms. For each polymorphic site the allelic discrimination assay uses two allele specific probes labeled with a different fluorescent dye at their 5′ ends but with a common quenching agent at their 3′ ends. Both probes have a 3′ phosphate group so that Taq polymerase cannot add nucleotides to them. The allele specific probes comprise the sequence encompassing the polymorphic site and differ in the sequence at this site. The allele specific probes are capable of hybridizing without mismatches to the appropriate site.

The allele specific probes are used in conjunction with two primers, one of which hybridizes to the template 5′ of the two specific probes, while the other hybridizes to the template 3′ of the two probes. If the allele corresponding to one of the specific probes is present, the specific probe will hybridize perfectly to the template. The Taq polymerase, extending the 5′ primer, will then remove the nucleotides from the specific probe, releasing both the fluorescent dye and the quenching agent. This will result in an increase in the fluorescence from the dye no longer in close proximity to the quenching agent.

If the allele specific probe hybridizes to the other allele the mismatch at the polymorphic site will inhibit the 5′ to 3′ endonuclease activity of Taq and hence prevent release of the fluorescent dye.

The ABI7700 sequence detection system is used to measure the increase in the fluorescence from each specific dye at the end of the thermal cycling PCR directly in PCR reaction tubes. The information from the reactions is then analyzed. If an individual is homozygous for a particular allele only fluorescence corresponding to the dye from that specific probe will be released, but if the individual is heterozygous, then both dyes will fluoresce.

The genotypes of the individuals are then correlated with their phenotypic response to treatment with a pharmaceutical compound intended for use as an aid in 7weight loss (e.g., norepinephrine reuptake inhibitors, dopamine reuptake inhibitors). Measured outcomes may include change over time in absolute weight, change in BMI score, change in a Food Craving Index, and change in body measurements; and/or may further include measurement of cardiovascular function such as heart rate, blood pressure, etc. Additionally, the occurrence of adverse events may be tabulated. Phenotypic responses (including desired outcomes, occurrence of adverse events, or significant changes in cardiovascular function) that vary among the genetic subpopulations are identified.

That which is claimed is:

1. A method of screening a human subject as an aid in predicting response to weight loss treatment with a norepinephrine reuptake inhibitor, comprising:
in a human subject in need of medical weight loss treatment, genotyping said subject at a polymorphic NET1 locus, where one form of said polymorphic locus has been associated with increased weight loss in response to treatment with a norepinephrine reuptake inhibitor, compared to weight loss associated with other polymorphic forms of the locus.

2. A method according to claim 1 where said NET1 locus is NET1 G155A locus.

3. A method according to claim 2 where said NET1 genotype is detected by screening for genetic markers in linkage disequilibrium with NET1 G155A alleles.

4. A method according to claim 1 where said NET1 locus is NET1 T342C.

5. A method according to claim 4 where said NET1 genotype is detected by screening for genetic markers in linkage disequilibrium with NET1 T342C alleles.

6. A method according to claim 1 where said NET1 locus is NET1 C120A locus.

7. A method according to claim 6 where said NET1 genotype is detected by screening for genetic markers in linkage disequilibrium with NET1 C120A alleles.

8. A method according to claim 1 where said norepinephrine reuptake inhibitor also is a reuptake inhibitor for a monoamine selected from dopamine and serotonin.

9. A method according to claim 1 where said norepinephrine reuptake inhibitor is GW320659.

10. A method according to claim 1 where said subject has a body mass index (BMI) of at least about 25 kg/m².

11. A method according to claim 1 where said subject has a body mass index (BMI) of at least about 30 kg/m².

12. A method according to claim 1 further comprising administering said norepinephrine reuptake inhibitor to said subject when said subject is determined to have a NET1 genotype associated with increased weight loss in response to treatment with said norepinephrine reuptake inhibitor.

13. A method according to claim 12 where said NET1 genotype associated with increased weight loss is NET1 T342 (C,C).

14. A method according to claim 12 where said NET1 genotype associated with increased weight loss is NET1 G155A (A,A).

15. A method according to claim 12 where said NET1 genotype associated with increased weight loss is NET1 C120A (A,A).

16. A method according to claim 1 wherein said subject has not previously been treated with a norepinephrine reuptake inhibitor for weight loss.

17. A method according to claim 1 wherein said subject has previously been treated with a norepinephrine reuptake inhibitor for weight loss.

18. A method of screening a human subject as an aid in predicting response to weight loss treatment with a dopamine reuptake inhibitor, comprising:
in a human subject in need of medical weight loss treatment, genotyping said subject at a polymorphic DAT1 locus, where one form of said polymorphic locus has been associated with increased weight loss in response to treatment with a dopamine reuptake inhibitor, compared to weight loss associated with other polymorphic forms of the locus.

19. A method according to claim 18 where said DAT1 locus is DAT1 VNTR.

20. A method according to claim 19 where said DAT1 genotype is detected by screening for genetic markers in linkage disequilibrium with the DAT1 VNTR alleles.

21. A method according to claim 18 where said dopamine reuptake inhibitor also is a reuptake inhibitor for a monoamine selected from norepinephrine and serotonin.

22. A method according to claim 21 where said reuptake inhibitor is GW320659.

23. A method according to claim 18 where said subject has a body mass index (BMI) of at least about 25 kg/m².

24. A method according to claim 18 where said subject has a body mass index (BMI) of at least about 30 kg/m².

25. A method according to claim 18 where said DAT1 genotype associated with increased weight loss is selected from DAT1 VNTR (9,9) and DAT1 VNTR (9,10).

26. A method according to claim 18 further comprising administering said reuptake inhibitor for weight loss when said subject is determined to have a DAT1 genotype that has been associated with increased weight loss in response to treatment with said reuptake inhibitor for weight loss, compared to the weight loss expected in a general population receiving the same treatment.

27. A method according to claim 18 wherein said subject has not previously been treated with a dopamine reuptake inhibitor for weight loss.

28. A method according to claim 18 wherein said subject has previously been treated with a dopamine reuptake inhibitor for weight loss.

29. A method of screening a human subject as an aid in predicting response to weight loss treatment with a norepinephrine reuptake inhibitor, comprising:
in a human subject in need of medical weight loss treatment, genotyping said subject at a polymorphic NR1 locus, where one form of said polymorphic locus has been associated with increased weight loss in response to treatment with a norepinephrine reuptake inhibitor, compared to weight loss associated with other polymorphic forms of the locus.

30. A method according to claim 29 where said NR1 locus is NR1 G6435A.

31. A method according to claim 30 where said NR1 genotype is detected by screening for genetic markers in linkage disequilibrium with NR1 G6435A alleles.

32. A method according to claim 29 where said NR1 locus is NRLG1001C.

33. A method according to claim 32 where said NR1 genotype is detected by screening for genetic markers in linkage disequilibrium with NR1 G1001C alleles.

34. A method according to claim 29 where said norepinephrine reuptake inhibitor also is a reuptake inhibitor for a monoamine selected from dopamine and serotonin.

35. A method according to claim 29 where said norepinephrine reuptake inhibitor is GW320659.

36. A method according to claim 29 where said subject has a body mass index (BMI) of at least about 25 kg/m².

37. A method according to claim 29 where said subject has a body mass index (BMI) of at least about 30 kg/m².

38. A method according to claim 29 where said NR1 genotype associated with increased weight loss is selected from NR1 G1001C (G/T) and NR1 G6435A (A/A).

39. A method according to claim 29 further comprising administering said norepinephrine reuptake inhibitor for weight loss when said subject is determined to have a NET1 genotype that has been associated with increased weight loss in response to treatment with said norepinephrine reuptake inhibitor for weight loss, compared to the weight loss expected in a general population receiving the same treatment.

40. A method according to claim 29 wherein said subject has not previously been treated with a norepinephrine reuptake inhibitor for weight loss.

41. A method according to claim 29 wherein said subject has previously been treated with a norepinephrine reuptake inhibitor for weight loss.

42. A method of screening a human subject as an aid in predicting response to weight loss treatment with a norepinephrine reuptake inhibitor, comprising:
in a human subject in need of medical weight loss treatment, genotyping said subject at a polymorphic 5HTT locus, where one form of said polymorphic locus has been associated with increased weight loss in response to treatment with a norepinephrine reuptake inhibitor, compared to weight loss associated with other polymorphic forms of the locus.

43. A method according to claim 42 where said 5HTT locus is 5HTT G769T.

44. A method according to claim 43 where said 5HTT genotype is detected by screening for genetic markers in linkage disequilibrium with 5HTT G769T alleles.

45. A method according to claim 42 where said 5HTT locus is 5HTT G160A.

46. A method according to claim 45 where said 5HTT genotype is detected by screening for genetic markers in linkage disequilibrium with 5HTT GI 60A alleles.

47. A method according to claim 42 where said norepinephrine reuptake inhibitor also is a reuptake inhibitor for a monoamine selected from dopamine and serotonin.

48. A method according to claim 42 where said norepinephrine reuptake inhibitor is GW320659.

49. A method according to claim 42 where said subject has a body mass index (BMI) of at least about 25 kg/m².

50. A method according to claim 42 where said subject has a body mass index (BMI) of at least about 30 kg/m².

51. A method according to claim 42 where said 5HTT genotype associated with increased weight loss is selected from 5HTT G769T (G,G) and 5HTT G160A (A,A).

52. A method according to claim 42 further comprising administering said norepinephrine reuptake inhibitor for weight loss when said subject is determined to have a 5HTT genotype that has been associated with increased weight loss in response to treatment with said norepinephrine reuptake inhibitor for weight loss, compared to the weight loss expected in a general population receiving the same treatment.

53. A method according to claim 42 wherein said subject has not previously been treated with a norepinephrine reuptake inhibitor for weight loss.

54. A method according to claim 42 wherein said subject has previously been treated with a norepinephrine reuptake inhibitor for weight loss.

55. A method of treating a human subject with a norepinephrine reuptake inhibitor for weight loss, the method comprising, in a subject in need of medical treatment for weight loss:
(a) genotyping a subject at a polymorphic locus in a gene selected from NET1, DAT1, NR1 and 5HTT, where a genotype of said polymorphic locus has been associated with increased weight loss in response to treatment with a norepinephrine reuptake inhibitor, compared to weight loss associated with another polymorphic form of that locus; and
(b) administering said norepinephrine reuptake inhibitor to the subject if said genotype associated with increased weight loss is detected.

56. A method according to claim 55 where said genotype associated with increased weight loss is selected from NET1 G155A (A,A); NET1 T342C(C/C); NET1C120A (A/A); DAT1 VNTR (10,9); DAT VNTR (9,9); NR1 G1001C (G/T); NR1 G6435A (A/A); 5HTT G769 (1/1); 5HTT G160A (2,2).

57. A method according to claim 55 where said norepinephrine reuptake inhibitor also is a reuptake inhibitor for a monoamine selected from dopamine and serotonin.

58. A method according to claim 55 where said norepinephrine reuptake inhibitor is GW320659.

59. A method according to claim 55 where said subject has a body mass index (BMI) of at least about 25 kg/m².

60. A method according to claim 55 where said subject has a body mass index (BMI) of at least about 30 kg/m².

61. A method according to claim 55 where said subject has not previously been treated with a norepinephrine reuptake inhibitor.

62. A method according to claim 55 where said subject has previously been treated with a norepinephrine reuptake inhibitor.

63. A method of identifying human genotypes associated with increased weight loss in response to treatment with a neuronal monoamine reuptake inhibitor for weight loss, comprising:
a) in a population of test subjects in need of weight loss treatment, genotyping each test subject at a polymorphic locus in a gene selected from NET1, DAT1, NR1, and 5HTT;
b) administering to each subject an effective weight loss regime of a neuronal monoamine reuptake inhibitor selected from norepinephrine reuptake inhibitor, dopamine reuptake inhibitor, and serotonin reuptake inhibitor;
c) measuring weight loss in each subject; and
d) correlating the genotypes of the test subjects with the extent of weight loss, to identify genotypes associated with increased weight loss, compared to average weight loss in the population.

64. A method according to claim 63 wherein said reuptake inhibitor is administered prior to genotyping.

65. A method according to claim 63 wherein said reuptake inhibitor is administered after genotyping.

66. A method of screening a human subject as an aid in predicting response to treatment with a dopamine reuptake inhibitor, comprising:
in a subject in need of treatment with a dopamine reuptake inhibitor, genotyping said subject at a polymorphic MAOB locus, where one form of said polymorphic locus has been associated with increased diastolic blood pressure changes in response to a therapeutic regimen of said norepinephrine reuptake inhibitor, compared to diastolic blood pressure changes associated with other polymorphic forms of the locus.

67. A method according to claim 66 where said MAOB locus is MAOB G644A.

68. A method according to claim 67 where said MAOB genotype is detected by screening for genetic markers in linkage disequilibrium with MAOB alleles.

69. A method according to claim 66 where said reuptake inhibitor also is a reuptake inhibitor for a monoamine selected from norepinephrine and serotonin.

70. A method according to claim 66 where said reuptake inhibitor is GW320659.

71. A method according to claim 66 where said MAOB genotype associated with increased diastolic blood pressure is selected from MAOB G644A (G/G) and MAOB G644A (A/G).

72. A method according to claim 66 further comprising administering said reuptake inhibitor to said subject when a MAOB genotype that has not been associated with increased diastolic blood pressure in response to treatment with said reuptake inhibitor is detected.

73. A method according to claim 72 where said MAOB genotype is MAOB G644A (A,A).

74. A method according to claim 66 wherein said subject has not previously been treated with a dopamine reuptake inhibitor.

75. A method according to claim 66 wherein said subject has previously been treated with a dopamine reuptake inhibitor.

76. A method of screening a human subject as an aid in predicting response to treatment with a dopamine reuptake inhibitor, comprising:
in a subject in need of treatment with a norepinephrine reuptake inhibitor, genotyping said subject at a polymorphic DRD2 locus, where one form of said polymorphic locus has been associated with increased heart rate changes in response to a therapeutic regimen of said dopamine reuptake inhibitor, compared to heart rate changes associated with other polymorphic forms of the locus.

77. A method according to claim 76 where said DRD2 locus is DRD2 C12121T.

78. A method according to claim 76 where said DRD2 genotype is detected by screening for genetic markers in linkage disequilibrium with DRD2 alleles.

79. A method according to claim 76 where said reuptake inhibitor also is a reuptake inhibitor for a monoamine selected from norepinephrine and serotonin.

80. A method according to claim 76 where said reuptake inhibitor is GW320659.

81. A method according to claim 76 where said DRD2 genotype associated with increased heart rate is DRD2 C12121 T (T/T).

82. A method according to claim 76 further comprising administering said reuptake inhibitor to said subject when a DRD2 genotype that has not been associated with increased heart rate in response to treatment with said reuptake inhibitor is detected.

83. A method according to claim 82 where said DRD2 genotype is DRD2 C12121T (C/C) or (T/C).

84. A method according to claim 76 wherein said subject has not previously been treated with a dopamine reuptake inhibitor.

85. A method according to claim 76 wherein said subject has previously been treated with a dopamine reuptake inhibitor.

86. A method of screening a subject in need of weight loss treatment, as an aid in predicting weight loss in response to weight loss treatment with a norepinephrine reuptake inhibitor, comprising:
detecting the subject's genotype at a polymorphic locus selected from NET1 T342C, NET1 G155A, and NR1 G6435A;
where detection of a genotype selected from NET1 T342C(C/C), NET1 G155A (A/A) and NR1 G6435A (A/A) indicates the subject is likely to achieve greater weight loss in response to said treatment, compared to weight loss expected in subjects with alternate genotypes.

87. A method according to claim 86, further comprising administering a therapeutic weight-loss regime of a norepinephrine reuptake inhibitor to the subject when a genotype selected from NET1 T342C(C/C), NET1 G155A (A/A) and NR1 G6435A (A/A) is selected.

88. A method according to claim 86 where said norepinephrine reuptake inhibitor also is a reuptake inhibitor for a monoamine selected from dopamine and serotonin.

89. A method according to claim 86 where said norepinephrine reuptake inhibitor is GW320659.

90. A method of screening a subject in need of treatment with a dopamine reuptake inhibitor, as an aid in predicting heart rate increase in response to treatment with said norepinephrine reuptake inhibitor, comprising:
detecting the subject's genotype at the DRD2 C12121T polymorphic locus,
where detection of the DRD2 C12121T (T/T) allele indicates the subject is likely to experience a greater heart rate increase, compared to heart rate increase expected in subjects with alternate genotypes.

91. A method according to claim 90, further comprising administering a therapeutic regime of said reuptake inhibitor to the subject when the genotype detected is DRD2 C12121T (C/C).

92. A method according to claim 90 where said reuptake inhibitor also is a reuptake inhibitor for a monoamine selected from norepinephrine and serotonin.

93. A method according to claim 90 where said reuptake inhibitor is GW320659.

94. A method of screening a subject in need of treatment with a dopamine reuptake inhibitor, as an aid in predicting heart rate increase in response to treatment with said reuptake inhibitor, comprising:
detecting the subject's genotype at the DRD2 C12121T polymorphic locus and the MAOB G644A polymorphic locus,
where detection of the DRD2 C12121 T (T/T) allele and the MAOB G644A (G,G) allele indicates the subject is likely to experience a greater heart rate increase, compared to heart rate increase expected in subjects with alternate genotypes.

95. A method according to claim 94, further comprising administering a therapeutic regime of said reuptake inhibitor to the subject when the genotypes detected are not DRD2 C12121T (T/T) and MAOB G644A (G,G).

96. A method according to claim 94 where said reuptake inhibitor also is a reuptake inhibitor for a monoamine selected from norepinephrine and serotonin.

97. A method according to claim 94 where said reuptake inhibitor is GW320659.

98. A method of treating a population of more than one subject in need of weight loss treatment, comprising:
detecting, in each subject, the genotype at a polymorphic locus selected from NET1 T342C, NET1 G155A, and NR1 G6435A;
administering a therapeutic weight-loss regime of a norepinephrine reuptake inhibitor to subjects in which the detected genotype is selected from NET1 T342C (C/C), NET1 G155A (A/A) and NR1 G6435A (A/A).

99. A method according to claim 93 where said norepinephrine reuptake inhibitor also is a reuptake inhibitor for a monoamine selected from dopamine and serotonin.

100. A method according to claim 93 where said norepinephrine reuptake inhibitor is GW320659.

101. A method of treating a population of more than one subject in need of weight loss treatment, comprising:
detecting, in each subject, the genotype at the DRD 1 C12121 T polymorphic locus;
administering a therapeutic weight-loss regime of a dopamine reuptake inhibitor to subjects in which the detected genotype at the DRD2 C12121T locus is (C,T) or (C,C).

102. A method according to claim 101 where said reuptake inhibitor also is a reuptake inhibitor for a monoamine selected from norepinephrine and serotonin.

103. A method according to claim 101 where said reuptake inhibitor is GW320659.

104. A method of treating a population of more than one subject in need of weight loss treatment, comprising:
detecting, in each subject, the genotype at the DRD1 C12121T polymorphic locus;
detecting, in each subject, the genotype at a polymorphic locus selected from NET1 T342C, NET1 G155A, and NR1 G6435A;
administering a therapeutic weight-loss regime of a norepinephrine reuptake inhibitor to subjects having a genotype selected from NET1 T342C(C/C), NET1 G155A (A/A) and NR1 G6435A (A/A) but not having a DRD1 C12121T (C/C) genotype.

105. A method according to claim 104 where said norepinephrine reuptake inhibitor also is a reuptake inhibitor for a monoamine selected from dopamine and serotonin.

106. A method according to claim 104 where said norepinephrine reuptake inhibitor is GW320659.

107. A method of administering a norepinephrine reuptake inhibitor for medical weight loss treatment, to increase the average weight loss achieved, comprising:
from a starting population of subjects in need of medical treatment for weight loss, selecting a treatment population having an increased percentage of subjects, compared to the starting population, with a genotype selected from NET1 G155A (A,A); NET1 T342C(C/C); NET1C120A (A/A); DAT1 VNTR (9,9); DAT VNTR (10,9); NR1 G1001C (G/C); NR1 G6435A (A/A); 5HTT G769 (G/G); and 5HTT G160A (A/A);
administering a norepinephrine reuptake inhibitor to the subjects in said treatment population;
whereby the average weight loss achieved is increased in the treatment population compared to the average weight loss that would be expected to occur in the starting population.

108. A method according to claim 107 where said norepinephrine reuptake inhibitor is GW320659.

109. A method according to claim 107 where said subjects have not previously been treated with a norepinephrine reuptake inhibitor for weight loss.

110. A method according to claim 107 where said subjects have previously been treated with a norepinephrine reuptake inhibitor for weight loss.

111. A method of administering a dopamine reuptake inhibitor, to reduce the average change in diastolic blood pressure, comprising:
selecting a treatment population of subjects from a general population of subjects in need of medical treatment with a dopamine reuptake inhibitor, said selected subjects having an MAOB G644A (A/A) genotype;
administering a dopamine reuptake inhibitor to the subjects in said treatment population;
where the average change in diastolic blood pressure is reduced in the treatment population compared to that which would be expected in a general population of subjects treated with said dopamine reuptake inhibitor.

112. A method according to claim 111 where said reuptake inhibitor is GW320659.

113. A method according to claim 111 where said subjects have not previously been treated with a dopamine reuptake inhibitor.

114. A method according to claim 111 where said subjects have previously been treated with a dopamine reuptake inhibitor.

115. A method of administering a dopamine reuptake inhibitor, to reduce the average change in heart rate, comprising:
selecting a treatment population of subjects from a general population of subjects in need of medical treatment with a dopamine reuptake inhibitor, said selected subjects having a genotype selected from DRD2 C12121T (C/C) and DRD2 C12121T (T/C);
administering a dopamine reuptake inhibitor to the subjects in said treatment population;
where the average change in heart rate is reduced in the treatment population compared to that which would be expected in a general population of subjects treated with said dopamine reuptake inhibitor.

116. A method according to claim 115 where said dopamine reuptake inhibitor is GW320659.

117. A method according to claim 115 where said subjects have not previously been treated with a dopamine reuptake inhibitor for weight loss.

118. A method according to claim 115 where said subjects have previously been treated with a dopamine reuptake inhibitor for weight loss.

119. A method of administering GW320659 for medical weight loss treatment, to increase the average weight loss achieved, comprising:
from a starting population of subjects in need of medical treatment for weight loss, selecting a treatment population having an increased percentage of subjects, compared to the starting population, with a genotype selected from NET1 G155A (A,A); NET1 T342C(C/C); NET1C120A (A/A); DAT1 VNTR (9,9); DAT VNTR (10,9); NR1 G1001C (G/C); NR1 G6435A (A/A); 5HTT G769 (G/G); and 5HTT G160A (A/A);
administering an effective weight loss regime of GW320659 to the subjects in said treatment population;
whereby the average weight loss achieved is increased in the treatment population compared to the average weight loss that would be expected to occur in the starting population.

120. A method according to claim 119 where said subjects have not previously been treated with GW320659.

121. A method according to claim 119 where said subjects have previously been treated with GW320659.

122. A method according to claim 119, further comprising selecting a treatment population having an increased percentage of subjects, compared to the starting population, with a genotype selected from DRD2 C12121T (C/C), DRD2 C12121T (T/C), and MAOB G644A (A/A).

123. A method of administering GW320659 to reduce the average change in heart rate, comprising:
selecting a treatment population of subjects from a general population of subjects in need of medical treatment with GW320659, said selected subjects having a genotype selected from DRD2 C12121T (C/C) and DRD2 C12121T (T/C);
administering GW320659 to the subjects in said treatment population;
where the average change in heart rate is reduced in the treatment population compared to that which would be expected in a general population of subjects treated with GW320659.

124. A method of administering GW320659, to reduce the average change in diastolic blood pressure, comprising:
selecting a treatment population of subjects from a general population of subjects in need of medical treatment with GW320659, said selected subjects having an MAOB G644A (A/A) genotype;
administering GW320659 to the subjects in said treatment population;
where the average change in diastolic blood pressure is reduced in the treatment population compared to that which would be expected in a general population of subjects treated with GW320659.

125. A method of treating subjects in need of medical weight loss treatment, comprising administering GW320659 to subjects having a genotype selected from DRD2 C12121T (C/C), DRD2 C12121T (T/C) and MAOB G644A (A/A).

126. A method of treating subjects in need of medical weight loss treatment, comprising administering GW320659 to subjects having a genotype selected NET1 G155A (A,A); NET1 T342C(C/C); NET1C120A (A/A); DAT1 VNTR (9,9); DAT VNTR (10,9); NR1 G1001C (G/C); NR1 G6435A (A/A); 5HTT G769 (G/G); and 5HTT G160A (A/A).

127. A method of selecting a treatment population to receive pharmaceutical norepinephrine reuptake inhibitor treatment, in order to increase average efficacy of said norepinephrine reuptake inhibitor treatment, comprising:
(a) from a starting population of subjects in need of pharmaceutical treatment with a norepinephrine reuptake inhibitor, selecting a treatment population consisting of a plurality of subjects having a genotype selected from NET1 G155A (A,A), NET1 T342C(C/C), NET1C120A (A/A), NR1 G1001C (G/C), and NR1 G6435A (A/A); and
(b) administering said pharmaceutical treatment to said treatment population, whereby the efficacy of said pharmaceutical treatment is increased in said treatment population compared to that which would be expected in said starting population.

128. A method of selecting a treatment population to receive pharmaceutical dopamine reuptake inhibitor treatment, in order to increase average efficacy of said dopamine reuptake inhibitor treatment, comprising:
(a) from a starting population of subjects in need of pharmaceutical treatment with a dopamine reuptake inhibitor, selecting a treatment population consisting of a plurality of subjects having a genotype selected from DAT1 VNTR (9,9) and DAT VNTR (10,9); and
(b) administering said pharmaceutical treatment to said treatment population, whereby the efficacy of said pharmaceutical treatment is increased in said treatment population compared to that which would be expected in said starting population.

129. A method of selecting a treatment population to receive pharmaceutical treatment with GW320659, in order to increase average efficacy of said pharmaceutical treatment, comprising:
(a) from a starting population of subjects in need of pharmaceutical treatment with GW320659, selecting a treatment population consisting of a plurality of subjects having a genotype selected from NET1 G155A (A,A), NET1 T342C(C/C), NET1C120A (A/A), DAT1 VNTR (9,9), DAT VNTR (10,9), NR1 G1001C (G/C), NR1 G6435A (A/A), 5HTT G769 (G/G), and 5HTT G160A (A/A), and
(b) administering said pharmaceutical treatment to said treatment population, whereby the efficacy of said pharmaceutical treatment is increased in said treatment population compared to that which would be expected in said starting population.
